(19) United States
(12) Patent Application Publication
(10) Pub. No.: US 2020/0095327 A1
(43) Pub. Date: Mar. 26, 2020

(54) ANTIBODY HEAVY CHAIN VARIABLE DOMAINS TARGETING THE NKG2D RECEPTOR

(71) Applicant: Dragonfly Therapeutics, Inc., Waltham, MA (US)

(72) Inventors: Gregory P. Chang, Medford, MA (US); Ann F. Cheung, Lincoln, MA (US); William Haney, Wayland, MA (US); Bradley M. Lunde, Lebanon, NH (US); Bianka Prinz, Lebanon, NH (US)

(73) Assignee: Dragonfly Therapeutics, Inc., Waltham, MA (US)

(21) Appl. No.: 16/483,572

(22) PCT Filed: Feb. 8, 2018
(86) PCT No.: PCT/US2018/017474

§ 371 Date: Aug. 5, 2019

Related U.S. Application Data
(60) Provisional application No. 62/456,544, filed on Feb. 8, 2017.

(51) Int. Cl. C07K 16/28; A61P 35/00; C07K 16/32; A61P 35/02

(52) U.S. Cl. CPC C07K 16/2851; A61K 2039/505; A61P 35/00; C07K 16/2803; C07K 16/32; C07K 16/2887; A61P 35/02; C07K 2317/565; C07K 2317/524; C07K 2317/526; C07K 2317/53; C07K 2317/522; C07K 2317/64; C07K 2317/75; C07K 2317/76; C07K 2317/33; C07K 2317/92; C07K 2317/94; C07K 2317/31; C07K 2317/622; C07K 2317/73; C07K 16/283

(57) ABSTRACT

Antibody heavy chain variable domains that can be paired with any of a variety of antibody light chain variable domains to form an antigen binding site targeting the NKG2D receptor on natural killer cells are described. Also described are antibodies and antigen-binding sites that compete for binding to NKG2D. Related pharmaceutical compositions and therapeutic methods, including for the treatment of cancer, are also described.

### CROSS-REFERENCE TO RELATED APPLICATIONS

This application claims the benefit of and priority to U.S. Provisional Patent Application No. 62/456,544, filed Feb. 8, 2017, the entire contents of which are incorporated by reference herein for all purposes.

### SEQUENCE LISTING

The instant application contains a Sequence Listing which has been submitted electronically in ASCII format and is hereby incorporated by reference in its entirety. Said ASCII copy, created on Feb. 6, 2018, is named DFY-002PC_SL.txt and is 55,870 bytes in size.

### FIELD OF THE INVENTION

The invention provides proteins with antibody heavy chain variable domains that can be paired with any of a variety of antibody light chain variable domains to form an antigen binding site targeting the NKG2D receptor on natural killer cells, pharmaceutical compositions comprising such proteins, and therapeutic methods using such proteins and pharmaceutical compositions, including for the treatment of cancer.

### BACKGROUND

Cancer continues to be a significant health problem despite the substantial research efforts and scientific advances reported in the literature for treating this disease. Some of the most frequently diagnosed cancers include prostate cancer, breast cancer, and lung cancer. Prostate cancer is the most common form of cancer in men. Breast cancer remains a leading cause of death in women. Current treatment options for these cancers are not effective for all patients and/or can have substantial adverse side effects. Other types of cancer also remain challenging to treat using existing therapeutic options.

Cancer immunotherapies are desirable because they are highly specific and can facilitate destruction of cancer cells using the patient's own immune system. Fusion proteins such as bi-specific T-cell engagers are cancer immunotherapies described in the literature that bind to tumor cells and T-cells to facilitate destruction of tumor cells. Antibodies that bind to certain tumor-associated antigens and to certain immune cells have been described in the literature. See, for example WO 2016/134371 and WO 2015/095412.

Natural killer (NK) cells are a component of the innate immune system and make up approximately 15% of circulating lymphocytes. NK cells infiltrate virtually all tissues and were originally characterized by their ability to kill tumor cells effectively without the need for prior sensitization. Activated NK cells kill target cells by means similar to cytotoxic T cells—i.e. via cytolytic granules that contain perform and granzymes as well as via death receptor pathways. Activated NK cells also secrete inflammatory cytokines such as IFN-gamma and chemokines that promote the recruitment of other leukocytes to the target tissue.

NK cells respond to signals through a variety of activating and inhibitory receptors on their surface, and the overall sensitivity of NK cells to activation depends on the sum of stimulatory and inhibitory signals. NKG2D is a type-II transmembrane protein that is expressed by essentially all natural killer cells where NKG2D serves as an activating receptor. NKG2D is also be found on T cells where it acts as a costimulatory receptor. The ability to modulate NK cell function via NKG2D is useful in various therapeutic contexts including malignancy.

### SUMMARY

Antibodies to NKG2D have been identified that provide important advantages in the design of therapeutic agents. For example, some of these antibodies do not merely bind human NKG2D, but have one or more further advantages such as the ability to agonize the receptor; the ability to compete with a natural ligand for binding to the receptor; and/or the ability to cross-react with NKG2D from other species such as mouse and/or cynomolgus monkey. These advantages can be achieved across a range of affinities for NKG2D and, in some cases, with excellent thermostability. Moreover, certain of the antibody heavy chains can maintain these properties even when paired with any of a variety of antibody light chains.

Accordingly, one aspect of the invention relates to an antibody heavy chain variable domain at least 90% identical to the amino acid sequence QVQLQQWGAGLLKPSETLSLTCAVYGGSFSGYYWSWIRQPPGKGLEWIGEIDHSGST NYNPSLKSRVTISVDTSKNQFSLKLSSVTAADTAVYYCARARGPWSFDPWGQGTLV TVSS (SEQ ID NO:1). In some instances, the antibody heavy chain variable domain is at least 95% identical to SEQ ID NO:1. In some embodiments, the heavy chain variable domain incorporates amino acid sequences GSFSGYYWS (SEQ ID NO:2) as the first complementarity-determining region (“CDR”), EIDHSGSTNYNPSLKS (SEQ ID NO:3) as the second CDR, and ARARGPWSFDP (SEQ ID NO:4) as the third CDR. In certain other embodiments, the heavy chain variable domain incorporates amino acid sequences SEQ ID NO:2 as the first CDR, SEQ ID NO:3 as the second CDR, and ARARGPWGFDP (SEQ ID NO:5) as the third CDR.

An antibody heavy chain variable domain of the invention can optionally be coupled to an amino acid sequence at least 90% identical to an antibody constant region, such as an IgG constant region including hinge, CH2, CH3 domains or CH1, hinge, CH2, and CH3 domains. Most often, the amino acid sequence is at least 90% identical to a human antibody constant region, such as an IgG1 constant region, an IgG2 constant region, IgG3 constant region, or IgG4 constant region, but amino acid sequences at least 90% identical to an antibody constant region from another mammal, such as dog, cat, mouse, or horse, for example, are also envisioned. One or more mutations as compared to human IgG1 can be incorporated, for example at Q347, Y349, L351, S354, E356, E357, K360, Q362, S364, T366, L368, K370, N390, K392, T394, D399, S400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, Y407A, Y407I, Y407V, K409F, K409W, K409D, T411D, T411E, K439D, and K439E.

In certain embodiments, mutations that can be included into the CH1 of a human IgG1 constant region may be at amino acid V125, F126, P127, T135, T139, A140, F170, P171, and/or V173. In certain embodiments, mutations that can be included into the Cκ of a human IgG1 constant region may be at amino acid E123, F116, S176, V163, S174, and/or T164.

Typically, one of the heavy chain variable domains described herein is combined with a light chain variable domain to form an antigen-binding site capable of binding NKG2D. As the heavy chain variable domain has proven capable of forming such an antigen-binding site with a variety of light chain variable domains, the specific light chain variable domain may be among those described herein; may be at least 50%, 55%, 60%, 65%, 70%, 75%, 80%, 85%, 90%, or 95% identical to one of those described herein, or may be largely unrelated. In certain embodiments, the light chain variable domain is one that can also pair with a different heavy chain variable domain to form an antigen-binding site specific for a tumor-associated antigen, such as EpCAM, BCMA, CD33, HER2, CD2, CD3, CD8, CD10, CD19, CD20, CD21, CD22, CD23, CD24, CD25, CD30, CD37, CD38, CD40, CD45RO, CD48, CD52, CD55, CD59, CD70, CD74, CD80, CD86, CD138, CD147, HLA-DR, CSAp, CA-125, TAG-72, EFGR/ERBB1, IGF1R, HER2, HER3, HER4, IGF-1R, c-Met, PDGFR, MUC1, MUC2, MUC3, MUC4, TNFR1, TNFR2, NGFR, TRAILR1, TRAILR2, Fas (CD95), DR3, DR4, DR5, DR6, VEGF, PIGF, tenascin, ED-B fibronectin, PSA, and IL-6, MAGE-A3, B7.1, B7.2, CTLA4 or PD1.

When a heavy chain variable domain is combined with a light chain variable domain to form an antigen-binding site capable of binding NKG2D, the antigen-binding site can be in the context of, for example, a typical antibody structure with two identical heavy chains and two identical light chains, forming a pair of antigen-binding sites capable of binding NKG2D; a bi-specific, tri-specific, tetra-specific, or other multi-specific antibody; or a smaller structure such as an scFv (in which the heavy chain variable domain is linked to the light chain variable domain).

Another aspect of the invention relates to antigen-binding sites that bind both to mouse and human (and, optionally, cynomolgus monkey) NKG2D. In one embodiment, the antigen-binding site competes for binding with an antibody having an antibody heavy chain having the amino acid sequence of SEQ ID NO:6 and an antibody light chain having the amino acid sequence of SEQ ID NO:7. In another embodiment, the antigen-binding site competes for binding with an antibody heavy chain having the amino acid sequence of SEQ ID NO:8 and an antibody light chain having the amino acid sequence of SEQ ID NO:9. In another embodiment, the antigen-binding site competes for binding with an antibody heavy chain having the amino acid sequence of SEQ ID NO:10 and an antibody light chain having the amino acid sequence of SEQ ID NO:11.

In certain embodiments, the antigen-binding site is in a protein that also includes a separate antigen-binding site that binds a tumor-associated antigen, which may permit the protein to simultaneously interact with an NK cell and a tumor. In some embodiments, the antigen-binding site is in a protein that is also capable of binding CD16, such as through an additional antigen-binding site or through an antibody constant region, such as an IgG1 constant region (which may optionally incorporate one or more mutations affecting, for example, effector activity or CD16 binding affinity).

Another aspect of the invention provides a method of treating cancer in a patient. The method comprises administering to a patient in need thereof a therapeutically effective amount of a protein described herein to treat the cancer. Exemplary cancers for treatment using the proteins include, for example, a carcinoma that expresses epithelial cell adhesion molecule (EpCAM).

### BRIEF DESCRIPTION OF THE DRAWINGS

FIG. 1 is a representation of the heterodimeric, multi-specific antibody. NKG2D binding domain (right arm), tumor antigen binding domain (left arm).

FIG. 2 is a representation of the heterodimeric, multi-specific antibody. NKG2D binding domain—scFv (right arm); tumor antigen binding domain (left arm).

FIG. 3 is a representation of a TriNKET in the Triomab form, which is a trifunctional, bispecific antibody that maintains an IgG-like shape. This chimera consists of two half antibodies, each with one light and one heavy chain, that originate from two parental antibodies. Triomab form may be an heterodimeric construct containing ½ of rat antibody and ½ of mouse antibody.

FIG. 4 is a representation of a TriNKET in the KiH Common Light Chain (LC) form, which involves the knobs-into-holes (KIHs) technology. KiH is a heterodimer containing 2 Fabs binding to target 1 and 2, and an F_{C} stabilized by heterodimerization mutations. TriNKET in the KiH format may be an heterodimeric construct with 2 fabs binding to target 1 and target 2, containing 2 different heavy chains and a common light chain that pairs with both HC.

FIG. 5 is a representation of a TriNKET in the dual-variable domain immunoglobulin (DVD-Ig™) form, which combines the target binding domains of two monoclonal antibodies via flexible naturally occurring linkers, and yields a tetravalent IgG-like molecule. DVD-Ig™ is an homodimeric construct where variable domain targeting antigen 2 is fused to the N terminus of variable domain of Fab targeting antigen 1 Construct contains normal F_{C}.

FIG. 6 is a representation of a TriNKET in the Orthogonal Fab interface (Ortho-Fab) form, which is an heterodimeric construct that contains 2 Fabs binding to target1 and target 2 fused to F_{C}. LC-HC pairing is ensured by orthogonal interface. Heterodimerization is ensured by mutations in the F_{C}.

FIG. 7 is a representation of a TrinKET in the 2 in 1Ig format.

FIG. 8 is a representation of a TriNKET in the ES form, which is an heterodimeric construct containing 2 different Fabs binding to target 1 and target 2 fused to the F_{C}. Heterodimerization is ensured by electrostatic steering mutations in the F_{C}.

FIG. 9 is a representation of a TriNKET in the Fab Arm Exchange form: antibodies that exchange Fab arms by swapping a heavy chain and attached light chain (half-molecule) with a heavy-light chain pair from another molecule, resulting in bispecific antibodies. Fab Arm Exchange form (cFae) is a heterodimer containing 2 Fabs binding to target 1 and 2, and an F_{C} stabilized by heterodimerization mutations.

FIG. 10 is a representation of a TriNKET in the SEED Body form, which is an heterodimer containing 2 Fabs binding to target 1 and 2, and an F_{C} stabilized by heterodimerization mutations.

FIG. 11 is a representation of a TriNKET in the LuZ-Y form, in which leucine zipper is used to induce heterodimerization of two different HCs. LuZ-Y form is a heterodimer containing 2 different scFabs binding to target 1 and 2, fused to F_{C}. Heterodimerization is ensured through leucine zipper motifs fused to C-terminus of F_{C}.

FIG. 12 is a representation of a TriNKET in the Cov-X-Body form.

FIGS. 13A-13B are representations of TriNKETs in the κλ-Body forms, which are an heterodimeric constructs with 2 different Fabs fused to F_{C} stabilized by heterodimerization mutations: Fab1 targeting antigen 1 contains kappa LC, while second Fab targeting antigen 2 contains lambda LC. FIG. 13A is an exemplary representation of one form of a κλ-Body; FIG. 13B is an exemplary representation of another κλ-Body.

FIG. 14 is a graph demonstrating the binding affinity of NKG2D binding domains (listed as clones) to human recombinant NKG2D in an ELISA assay.

FIG. 15 is a graph demonstrating the binding affinity of NKG2D binding domains (listed as clones) to cynomolgus recombinant NKG2D in an ELISA assay.

FIG. 16 is a graph demonstrating the binding affinity of NKG2D binding domains (listed as clones) to mouse recombinant NKG2D in an ELISA assay.

FIG. 17 is a graph demonstrating the binding of NKG2D binding domains (listed as clones) to EL4 cells expressing human NKG2D by flow cytometry showing mean fluorescence intensity (MFI) fold over background.

FIG. 18 is a graph demonstrating the binding of NKG2D binding domains (listed as clones) to EL4 cells expressing mouse NKG2D by flow cytometry showing mean fluorescence intensity (MFI) fold over background.

FIG. 19 is a graph demonstrating specific binding affinity of NKG2D binding domains (listed as clones) to recombinant human NKG2D-Fc by competing with natural ligand ULBP-6.

FIG. 20 is a graph demonstrating specific binding affinity of NKG2D binding domains (listed as clones) to recombinant human NKG2D-Fc by competing with natural ligand MICA.

FIG. 21 is a graph demonstrating specific binding affinity of NKG2D binding domains (listed as clones) to recombinant mouse NKG2D-Fc by competing with natural ligand Rae-1 delta.

FIG. 22 is a graph showing activation of human NKG2D by NKG2D binding domains (listed as clones) by quantifying the percentage of TNF-alpha positive cells which express human NKG2D-CD3 zeta fusion proteins.

FIG. 23 is a graph showing activation of mouse NKG2D by NKG2D binding domains (listed as clones) by quantifying the percentage of TNF-alpha positive cells which express mouse NKG2D-CD3 zeta fusion proteins.

FIG. 24 is a graph showing activation of human NK cells by NKG2D binding domains (listed as clones).

FIG. 25 is a graph showing activation of human NK cells by NKG2D binding domains (listed as clones).

FIG. 26 is a graph showing activation of mouse NK cells by NKG2D binding domains (listed as clones).

FIG. 27 is a graph showing activation of mouse NK cells by NKG2D binding domains (listed as clones).

FIG. 28 is a graph showing the cytotoxic effect of NKG2D binding domains (listed as clones) on tumor cells.

FIG. 29 is a graph showing the melting temperature of NKG2D binding domains (listed as clones) measured by differential scanning fluorimetry.

FIG. 30 is a bar graph showing tri-specific binding in one molecule is important for maximal NK cell activity.

FIG. 31 is a binding profile of CD33-targeting TriNKETs to NKG2D expressed on EL4 cells. FIG. 31 shows binding of the two TriNKETs when a CD33-binding domain is used as the second targeting arm.

FIG. 32 is a binding profile of HER2-targeting TriNKETs to NKG2D expressed on EL4 cells. FIG. 32 shows the same two NKG2D-binding domains now paired with a HER2 second targeting arm.

FIG. 33 is a histogram of CD20-targeting TriNKETs that bind to NKG2D expressed on EL4 cells. Unstained EL4 cells were used a negative control for fluorescence signal. Unstained: filled; CD20-TriNKET-F04: solid line; CD20-TriNKET-C26: dashed line.

FIG. 34 is a binding profile of CD33-targeting TriNKETs to CD33 expressed on MV4-11 human AML cells.

FIG. 35 is a binding profile of HER2-targeting TriNKETs to HER2 expressed on human 786-O renal cell carcinoma cells.

FIG. 36 is a histogram of CD20-targeting TriNKETs that bind to CD20 expressed on Raji human lymphoma cells. Unstained cells were used a negative control for fluorescence signal. Unstained: filled; CD20-TriNKET-F04: solid line; CD20-TriNKET-C26: dashed line.

FIGS. 37A-37C are bar graphs of synergistic activation of NK cells using CD16 and NKG2D. FIG. 37A demonstrates levels of CD107a; FIG. 37B demonstrates levels of IFNγ; FIG. 37C demonstrates levels of CD107a. Graphs indicate the mean (n=2)±SD. Data are representative of five independent experiments using five different healthy donors.

FIG. 38 is a bar graph showing activation of NK cells using TriNKETs targeting NKG2D and CD16. Antibodies tested were of human IgG1 isotypes. Graphs indicate the mean (n=2)±SD.

FIGS. 39A-39C are bar graphs demonstrating that TriNKETs and trastuzumab were able to activate primary human NK cells in co-culture with HER2-positive human tumor cells, indicated by an increase in CD107a degranulation and IFNγ cytokine production. Compared to the monoclonal antibody trastuzumab, both TriNKETs showed superior activation of human NK cells with a variety of human HER2 cancer cells. FIG. 39A shows that human NK cells are activated by TriNKETs when cultured with SkBr-3 cells. FIG. 39B shows that human NK cells are activated by TriNKETs when cultured with Colo201 cells. FIG. 39C shows that human NK cell are activated by TriNKETs when cultured with HCC1954 cells.

FIGS. 40A-40B are line graphs demonstrating TriNKET-mediated activation of rested or IL-2-activated human NK cells in co-culture with the CD33-expressing human AML cell line MV4-11. FIG. 40A shows TriNKET-mediated activation of resting human NK cells. FIG. 40B shows TriNKET-mediated activation of IL-2-activated human NK cells from the same donor.

FIGS. 41A-41B are graphs demonstrating TriNKET enhancement of cytotoxic activity using IL-2-activated and rested human NK cells. FIG. 41A shows percent specific lysis of SkBr-3 tumor cells by rested human NK cells. FIG. 41B shows percent specific lysis of SkBr-3 tumor cells by IL-2-activated human NK cells.

FIGS. 42A-42B are graphs demonstrating TriNKETs provide the greater advantage against HER2 medium and low cancers compared to trastuzumab. FIG. 42A shows activated human NK cell killing of HER2 high-SkBr-3 tumor cells. FIG. 42B shows human NK cell killing of HER2 low-786-O tumor cells. TriNKETs provide a greater advantage compared to trastuzumab against cancer cells with low HER2 expression.

FIGS. 43A-43C are histograms showing that the expression of the high-affinity FcRγI (CD64) on three human AML cells lines, Molm-13 cell line (FIG. 43A), Mv4-11 cell line (FIG. 43B), and THP-1 cell line (FIG. 43C).

FIGS. 44A-44B are line graphs of monoclonal antibody or TriNKET mediated activation of human NK cells in co-culture with either Molm-13 (FIG. 44B) or THP-1 (FIG. 44A) cells.

FIGS. 45A-45C are line graphs of human NK cytotoxicity assays using the three human AML cell lines as targets. FIG. 45A shows that Mv4-11 cells, which express CD64, but at a lower level than THP-1, showed reduced efficacy with the monoclonal anti-CD33.

FIG. 45B demonstrates that a monoclonal antibody against CD33 shows good efficacy against Molm-13 cells, which do not express CD64. FIG. 45C demonstrates that THP-1 cells showed no effect with monoclonal anti-CD33 alone. The identities of the line graphs noted in FIG. 45C are also applicable to the line graphs in FIGS. 45A-45B.

FIGS. 46A & 46B are bar graphs showing B cells from a health donor are sensitive to TriNKET-mediated lysis.

FIGS. 46C & 46D are bar graphs showing myeloid cells are resistant to TriNKET-mediated lysis.

FIG. 47 are line graphs of TriNKETs-mediated hPBMC killing of SkBr-3 tumor cells in long-term co-cultures.

FIG. 48 is a flowchart of study design of RMA/S-HER2 subcutaneous SC2.2 efficacy.

FIG. 49 are line graphs showing that SC2.2 has no effect on subcutaneous RMA/S-HER2 tumor growth.

FIGS. 50A-50B are graphs showing in vitro binding by mcFAE-C26.99 TriNKET. 60 μg/mL of indicated antibodies with four-fold dilutions were added to 2×10⁵ B16F10 tumor cells (FIG. 50A) or EL4-mNKG2D cells (FIG. 50B). Binding was assessed using a goat anti-mouse PE secondary antibody followed by flow cytometric analysis.

FIG. 51 is a graph showing increased NK cytotoxicity mediated by mcFAE-C26.99 TriNKET.

FIGS. 52A-52B show the anti-tumor efficacy of mcFAE-C26.99 TriNKET in B16F10 s.c. models. Mice were treated intraperitoneally with (FIG. 52A) isotype control mouse IgG2a mab C1.18.4 and mouse anti-Tyrp-1 monoclonal antibody or (FIG. 52B) isotype control mouse IgG2a mab C1.18.4 and mcFAE-C26.99 TriNKET, injected at a dose of 150 μg (days 6, 8, 10, 12, 14, 16, and 21). Tumor growth was assessed for 28 days. Graphs show tumor growth curves of individual mice.

FIGS. 53A-53B show anti-tumor efficacy of mcFAE-C26.99 TriNKET in B 16F10 i.v. models. FIG. 53A represents tumor burden when antibodies were administered at a 150-μg dose (days 4, 6, 8, 11, 13, 15). FIG. 53B represents tumor burden when antibodies were administered at a 150-μg dose (days 7, 9, 11, 13, 15). 18 days after tumor challenge, mice were euthanized and surface lung metastases were scored.

FIG. 54 is bar graph showing that human NK cells are activated by TriNKETs when cultured with CD20+ Raji cells.

FIG. 55 is an Oasc-Fab heterodimeric construct that includes Fab binding to target 1 and scFab binding to target 2 fused to F_{C}. Heterodimerization is ensured by mutations in the F_{C}.

FIG. 56 is a DuetMab, which is an heterodimeric construct containing 2 different Fabs binding to antigen 1 and 2 and F_{C} stabilized by heterodimerization mutations. Fab 1 and 2 contain differential S-S bridges that ensure correct LC and HC pairing.

FIG. 57 is a CrossmAb, which is an heterodimeric construct with 2 different Fabs binding to target 1 and 2 fused to Fc stabilized by heterodimerization. CL and CH1 domains and VH and VL domains are switched, e.g., CH1 is fused in-line with VL, while CL is fused in-line with VH.

FIG. 58 is a Fit-Ig, which is an homodimeric constructs where Fab binding to antigen 2 is fused to the N terminus of HC of Fab that binds to antigen 1. The construct contains wild-type F_{C}.

### DETAILED DESCRIPTION

The invention provides proteins with antibody heavy chain variable domains that can be paired with any of a variety of antibody light chain variable domains to form an antigen binding site targeting the NKG2D receptor on natural killer cells, pharmaceutical compositions comprising such proteins, and therapeutic methods using such proteins and pharmaceutical compositions, including for the treatment of cancer. Various aspects of the invention are set forth below in sections; however, aspects of the invention described in one particular section are not to be limited to any particular section.

To facilitate an understanding of the present invention, a number of terms and phrases are defined below.

The terms “a” and “an” as used herein mean “one or more” and include the plural unless the context is inappropriate.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably include humans.

As used herein, the term “antigen-binding site” refers to the part of the immunoglobulin molecule that participates in antigen binding. In human antibodies, the antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FRs”. Thus the term “FR” refers to amino acid sequences which are naturally found between and adjacent to hypervariable regions in immunoglobulins. In a human antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” In certain animals, such as camels and cartilaginous fish, the antigen-binding site is formed by a single antibody chain providing a “single domain antibody.” Antigen-binding sites can exist in an intact antibody, in an antigen-binding fragment of an antibody that retains the antigen-binding surface, or in a recombinant polypeptide such as an scFv, using a peptide linker to connect the heavy chain variable domain to the light chain variable domain in a single polypeptide.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, Pa. [1975].

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### I. Antibody Heavy Chain Variable Domains

The invention provides antigen-binding sites that bind NKG2D, and antigen heavy chain variable domains that can be used to create such antigen-binding sites.

For example, certain antibody heavy chain variable domains described below can be paired with any of a variety of antibody light chain variable domains to form an antigen binding site targeting the NKG2D receptor on natural killer cells. Binding of the protein to a tumor-associated antigen on a cancer cell brings the cancer cell into proximity to the natural killer cell, which facilitates destruction of the cancer cell by the natural killer cell either directly or indirectly. Further description of exemplary proteins is provided below.

Several combinations of antibody heavy and light chain variable domains forming antigen-binding sites capable of binding and agonizing the NKG2D receptor have now been identified and are provided in Table 1, below.

TABLE 1

| | Heavy chain variable region amino acid | Light chain variable region amino |
|---|---|---|
| Clones | sequence | acid sequence |
| ADI-27705 | QVQLQQWGAGLLKPSETLSLTCAV | DIQMTQSPSTLSASVGDRVTITC |
| | YGGSFSGYYWSWIRQPPGKGLEWIG | RASQSISSWLAWYQQKPGKAPK |
| | EIDHSGSTNYNPSLKSRVTISVDTSK | LLIYKASSLESGVPSRFSGSGSGT |
| | NQFSLKLSSVTAADTAVYYCARAR | EFTLTISSLQPDDFATYYCQQYN |
| | GPWSFDPWGQGTLVTVSS | SYPITFGGGTKVEIK |
| | (SEQ ID NO: 1) | (SEQ ID NO: 12) |
| ADI-27724 | QVQLQQWGAGLLKPSETLSLTCAV | EIVLTQSPGTLSLSPGERATLSCR |
| | YGGSFSGYYWSWIRQPPGKGLEWIG | ASQSVSSSYLAWYQQKPGQAPR |
| | EIDHSGSTNYNPSLKSRVTISVDTSK | LLIYGASSRATGIPDRFSGSGSGT |
| | NQFSLKLSSVTAADTAVYYCARAR | DFTLTISRLEPEDFAVYYCQQYG |
| | GPWSFDPWGQGTLVTVSS | SSPITFGGGTKVEIK |
| | (SEQ ID NO: 1) | (SEQ ID NO: 13) |
| ADI-27740 | QVQLQQWGAGLLKPSETLSLTCAV | DIQMTQSPSTLSASVGDRVTITC |
| (A40) | YGGSFSGYYWSWIRQPPGKGLEWIG | RASQSIGSWLAWYQQKPGKAP |
| | EIDHSGSTNYNPSLKSRVTISVDTSK | KLLIYKASSLESGVPSRFSGSGS |
| | NQFSLKLSSVTAADTAVYYCARAR | GTEFTLTISSLQPDDFATYYCQQ |
| | GPWSFDPWGQGTLVTVSS | YHSFYTFGGGTKVEIK |
| | (SEQ ID NO: 1) | (SEQ ID NO: 14) |
| ADI-27741 | QVQLQQWGAGLLKPSETLSLTCAV | DIQMTQSPSTLSASVGDRVTITC |
| | YGGSFSGYYWSWIRQPPGKGLEWIG | RASQSIGSWLAWYQQKPGKAP |
| | EIDHSGSTNYNPSLKSRVTISVDTSK | KLLIYKASSLESGVPSRFSGSGS |
| | NQFSLKLSSVTAADTAVYYCARAR | GTEFTLTISSLQPDDFATYYCQQ |
| | GPWSFDPWGQGTLVTVSS | SNSYYTFGGGTKVEIK |
| | (SEQ ID NO: 1) | (SEQ ID NO: 15) |
| ADI-27743 | QVQLQQWGAGLLKPSETLSLTCAV | DIQMTQSPSTLSASVGDRVTITC |
| | YGGSFSGYYWSWIRQPPGKGLEWIG | RASQSISSWLAWYQQKPGKAPK |
| | EIDHSGSTNYNPSLKSRVTISVDTSK | LLIYKASSLESGVPSRFSGSGSGT |
| | NQFSLKLSSVTAADTAVYYCARAR | EFTLTISSLQPDDFATYYCQQYN |
| | GPWSFDPWGQGTLVTVSS | SYPTFGGGTKVEIK |
| | (SEQ ID NO: 1) | (SEQ ID NO: 16) |
| ADI-28153 | QVQLQQWGAGLLKPSETLSLTCAV | ELQMTQSPSSLSASVGDRVTITC |
| | YGGSFSGYYWSWIRQPPGKGLEWIG | RTSQSISSYLNWYQQKPGQPPKL |
| | EIDHSGSTNYNPSLKSRVTISVDTSK | LIYWASTRESGVPDRFSGSGSGT |
| | NQFSLKLSSVTAADTAVYYCARAR | DFTLTISSLQPEDSATYYCQQSY |
| | GPWGFDPWGQGTLVTVSS | DIPYTFGQGTKLEIK |
| | (SEQ ID NO: 17) | (SEQ ID NO: 18) |
| ADI-28226 | QVQLQQWGAGLLKPSETLSLTCAV | DIQMTQSPSTLSASVGDRVTITC |
| (C26) | YGGSFSGYYWSWIRQPPGKGLEWIG | RASQSISSWLAWYQQKPGKAPK |
| | EIDHSGSTNYNPSLKSRVTISVDTSK | LLIYKASSLESGVPSRFSGSGSGT |
| | NQFSLKLSSVTAADTAVYYCARAR | EFTLTISSLQPDDFATYYCQQYG |
| | GPWSFDPWGQGTLVTVSS | SFPITFGGGTKVEIK |
| | (SEQ ID NO: 1) | (SEQ ID NO: 19) |
| ADI-28154 | QVQLQQWGAGLLKPSETLSLTCAV | DIQMTQSPSTLSASVGDRVTITC |
| | YGGSFSGYYWSWIRQPPGKGLEWIG | RASQSISSWLAWYQQKPGKAPK |
| | EIDHSGSTNYNPSLKSRVTISVDTSK | LLIYKASSLESGVPSRFSGSGSGT |
| | NQFSLKLSSVTAADTAVYYCARAR | DFTLTISSLQPDDFATYYCQQSK |
| | GPWSFDPWGQGTLVTVSS | EVPWTFGQGTKVEIK |
| | (SEQ ID NO: 1) | (SEQ ID NO: 20) |
| ADI-29399 | QVQLQQWGAGLLKPSETLSLTCAV | DIQMTQSPSTLSASVGDRVTITC |
| | YGGSFSGYYWSWIRQPPGKGLEWIG | RASQSISSWLAWYQQKPGKAPK |
| | EIDHSGSTNYNPSLKSRVTISVDTSK | LLIYKASSLESGVPSRFSGSGSGT |
| | NQFSLKLSSVTAADTAVYYCARAR | EFTLTISSLQPDDFATYYCQQYN |
| | GPWSFDPWGQGTLVTVSS | SFPTFGGGTKVEIK |
| | (SEQ ID NO: 1) | (SEQ ID NO: 21) |
| ADI-29401 | QVQLQQWGAGLLKPSETLSLTCAV | DIQMTQSPSTLSASVGDRVTITC |
| | YGGSFSGYYWSWIRQPPGKGLEWIG | RASQSIGSWLAWYQQKPGKAP |
| | EIDHSGSTNYNPSLKSRVTISVDTSK | KLLIYKASSLESGVPSRFSGSGS |
| | NQFSLKLSSVTAADTAVYYCARAR | GTEFTLTISSLQPDDFATYYCQQ |
| | GPWSFDPWGQGTLVTVSS | YDIYPTFGGGTKVEIK |
| | (SEQ ID NO: 1) | (SEQ ID NO: 22) |
| ADI-29403 | QVQLQQWGAGLLKPSETLSLTCAV | DIQMTQSPSTLSASVGDRVTITC |
| | YGGSFSGYYWSWIRQPPGKGLEWIG | RASQSISSWLAWYQQKPGKAPK |
| | EIDHSGSTNYNPSLKSRVTISVDTSK | LLIYKASSLESGVPSRFSGSGSGT |
| | NQFSLKLSSVTAADTAVYYCARAR | EFTLTISSLQPDDFATYYCQQYD |
| | GPWSFDPWGQGTLVTVSS | SYPTFGGGTKVEIK |
| | (SEQ ID NO: 1) | (SEQ ID NO: 23) |
| ADI-29405 | QVQLQQWGAGLLKPSETLSLTCAV | DIQMTQSPSTLSASVGDRVTITC |
| | YGGSFSGYYWSWIRQPPGKGLEWIG | RASQSISSWLAWYQQKPGKAPK |
| | EIDHSGSTNYNPSLKSRVTISVDTSK | LLIYKASSLESGVPSRFSGSGSGT |
| | NQFSLKLSSVTAADTAVYYCARAR | EFTLTISSLQPDDFATYYCQQYG |
| | GPWSFDPWGQGTLVTVSS | SFPTFGGGTKVEIK |
| | (SEQ ID NO: 1) | (SEQ ID NO: 24) |
| ADI-29407 | QVQLQQWGAGLLKPSETLSLTCAV | DIQMTQSPSTLSASVGDRVTITC |
| | YGGSFSGYYWSWIRQPPGKGLEWIG | RASQSISSWLAWYQQKPGKAPK |
| | EIDHSGSTNYNPSLKSRVTISVDTSK | LLIYKASSLESGVPSRFSGSGSGT |
| | NQFSLKLSSVTAADTAVYYCARAR | EFTLTISSLQPDDFATYYCQQYQ |
| | GPWSFDPWGQGTLVTVSS | SFPTFGGGTKVEIK |
| | (SEQ ID NO: 1) | (SEQ ID NO: 25) |
| ADI-29419 | QVQLQQWGAGLLKPSETLSLTCAV | DIQMTQSPSTLSASVGDRVTITC |
| | YGGSFSGYYWSWIRQPPGKGLEWIG | RASQSISSWLAWYQQKPGKAPK |
| | EIDHSGSTNYNPSLKSRVTISVDTSK | LLIYKASSLESGVPSRFSGSGSGT |
| | NQFSLKLSSVTAADTAVYYCARAR | EFTLTISSLQPDDFATYYCQQYS |
| | GPWSFDPWGQGTLVTVSS | SFSTFGGGTKVEIK |
| | (SEQ ID NO: 1) | (SEQ ID NO: 26) |
| ADI-29421 | QVQLQQWGAGLLKPSETLSLTCAV | DIQMTQSPSTLSASVGDRVTITC |
| | YGGSFSGYYWSWIRQPPGKGLEWIG | RASQSISSWLAWYQQKPGKAPK |
| | EIDHSGSTNYNPSLKSRVTISVDTSK | LLIYKASSLESGVPSRFSGSGSGT |
| | NQFSLKLSSVTAADTAVYYCARAR | EFTLTISSLQPDDFATYYCQQYE |
| | GPWSFDPWGQGTLVTVSS | SYSTFGGGTKVEIK |
| | (SEQ ID NO: 1) | (SEQ ID NO: 27) |
| ADI-29424 | QVQLQQWGAGLLKPSETLSLTCAV | DIQMTQSPSTLSASVGDRVTITC |
| | YGGSFSGYYWSWIRQPPGKGLEWIG | RASQSISSWLAWYQQKPGKAPK |
| | EIDHSGSTNYNPSLKSRVTISVDTSK | LLIYKASSLESGVPSRFSGSGSGT |
| | NQFSLKLSSVTAADTAVYYCARAR | EFTLTISSLQPDDFATYYCQQYD |
| | GPWSFDPWGQGTLVTVSS | SFITFGGGTKVEIK |
| | (SEQ ID NO: 1) | (SEQ ID NO: 28) |
| ADI-29425 | QVQLQQWGAGLLKPSETLSLTCAV | DIQMTQSPSTLSASVGDRVTITC |
| | YGGSFSGYYWSWIRQPPGKGLEWIG | RASQSISSWLAWYQQKPGKAPK |
| | EIDHSGSTNYNPSLKSRVTISVDTSK | LLIYKASSLESGVPSRFSGSGSGT |
| | NQFSLKLSSVTAADTAVYYCARAR | EFTLTISSLQPDDFATYYCQQYQ |
| | GPWSFDPWGQGTLVTVSS | SYPTFGGGTKVEIK |
| | (SEQ ID NO: 1) | (SEQ ID NO: 29) |
| ADI-29426 | QVQLQQWGAGLLKPSETLSLTCAV | DIQMTQSPSTLSASVGDRVTITC |
| | YGGSFSGYYWSWIRQPPGKGLEWIG | RASQSIGSWLAWYQQKPGKAP |
| | EIDHSGSTNYNPSLKSRVTISVDTSK | KLLIYKASSLESGVPSRFSGSGS |
| | NQFSLKLSSVTAADTAVYYCARAR | GTEFTLTISSLQPDDFATYYCQQ |
| | GPWSFDPWGQGTLVTVSS | YHSFPTFGGGTKVEIK |
| | (SEQ ID NO: 1) | (SEQ ID NO: 30) |
| ADI-29429 | QVQLQQWGAGLLKPSETLSLTCAV | DIQMTQSPSTLSASVGDRVTITC |
| | YGGSFSGYYWSWIRQPPGKGLEWIG | RASQSIGSWLAWYQQKPGKAP |
| | EIDHSGSTNYNPSLKSRVTISVDTSK | KLLIYKASSLESGVPSRFSGSGS |
| | NQFSLKLSSVTAADTAVYYCARAR | GTEFTLTISSLQPDDFATYYCQQ |
| | GPWSFDPWGQGTLVTVSS | YELYSYTFGGGTKVEIK |
| | (SEQ ID NO: 1) | (SEQ ID NO: 31) |
| ADI-29447 | QVQLQQWGAGLLKPSETLSLTCAV | DIQMTQSPSTLSASVGDRVTITC |
| (F47) | YGGSFSGYYWSWIRQPPGKGLEWIG | RASQSISSWLAWYQQKPGKAPK |
| | EIDHSGSTNYNPSLKSRVTISVDTSK | LLIYKASSLESGVPSRFSGSGSGT |
| | NQFSLKLSSVTAADTAVYYCARAR | EFTLTISSLQPDDFATYYCQQYD |
| | GPWSFDPWGQGTLVTVSS | TFITFGGGTKVEIK |
| | (SEQ ID NO: 1) | (SEQ ID NO: 32) |
| ADI-29404 | QVQLQQWGAGLLKPSETLSLTCAV | DIQMTQSPSTLSASVGDRVTITC |
| (F04) | YGGSFSGYYWSWIRQPPGKGLEWIG | RASQSISSWLAWYQQKPGKAPK |
| | EIDHSGSTNYNPSLKSRVTISVDTSK | LLIYKASSLESGVPSRFSGSGSGT |
| | NQFSLKLSSVTAADTAVYYCARAR | EFTLTISSLQPDDFATYYCEQYD |
| | GPWSFDPWGQGTLVTVSS | SYPTFGGGTKVEIK |
| | (SEQ ID NO: 1) | (SEQ ID NO: 44) |
| ADI-28200 | QVQLVQSGAEVKKPGSSVKVSCKA | DIVMTQSPDSLAVSLGERATINC |
| | SGGTFSSYAISWVRQAPGQGLEWM | ESSQSLLNSGNQKNYLTWYQQK |
| | GGIIPIFGTANYAQKFQGRVTITADE | PGQPPKPLIYWASTRESGVPDRF |
| | STSTAYMELSSLRSEDTAVYYCARR | SGSGSGTDFTLTISSLQAEDVAV |
| | GRKASGSFYYYYGMDVWGQGTTV | YYCQNDYSYPYTFGQGTKLEIK |
| | TVSS | (SEQ ID NO: 46) |
| | (SEQ ID NO: 45) | |

Importantly, each of the heavy chain variable domain amino acid sequences listed in Table 1 is at least 90% identical to the heavy chain variable domain amino acid sequence of SEQ ID NO:1. In contrast, the sequences of the light chain variable domains recited in Table 1 vary substantially, with the two most divergent sequences differing by 35% as shown in Table 2. Accordingly, starting from the antibody heavy chain variable domain amino acid sequences, it is possible to construct a variety of antigen-binding sites and multi-specific antibodies facilitating NKG2D binding, NK cell activation, and tumor cell killing, for example.

TABLE 2

| | | Percent Identity | | | | | | | | | | |
|---|---|---|---|---|---|---|---|---|---|---|---|---|
| | | 1 | 2 | 3 | 4 | 5 | 6 | 7 | 8 | 9 | 10 | 11 |
| Percent | 1 | | 74.8 | [symbol] | 97.2 | 100.8 | [symbol] | [symbol] | 94.4 | [symbol] | 97.2 | [symbol] |
| divergence | 2 | 33.8 | | 72.9 | 72.0 | [symbol] | 70.4 | 75.7 | 72.2 | 74.8 | [symbol] | 74.8 |
| | 3 | 3.8 | [symbol] | | 47.2 | 96.5 | 78.5 | 97.2 | [symbol] | 97.2 | 96.5 | [symbol] |
| | 4 | 3.8 | 35.1 | 2.9 | | 97.2 | 79.4 | [symbol] | [symbol] | [symbol] | [symbol] | [symbol] |
| | 5 | [symbol] | 30.8 | 3.8 | [symbol] | | 80.4 | 98.1 | 94.4 | 98.1 | 97.2 | [symbol] |
| | 6 | 22.5 | 37.7 | 25.4 | 24.1 | 22.8 | | 80.4 | [symbol] | 80.4 | [symbol] | [symbol] |
| | 7 | 1.9 | 29.4 | 2.9 | [symbol] | 1.9 | 22.8 | | [symbol] | 99.1 | [symbol] | [symbol] |
| | 8 | [symbol] | 34.7 | [symbol] | 6.9 | 5.8 | 20.1 | 5.8 | | [symbol] | [symbol] | 94.4 |
| | 9 | 0.9 | 30.8 | [symbol] | 3.8 | [symbol] | 22.8 | [symbol] | [symbol] | | [symbol] | [symbol] |
| | 10 | 2.9 | [symbol] | 3.8 | 3.8 | 2.9 | 24.1 | 3.8 | [symbol] | 3.8 | | [symbol] |
| | 11 | 0.9 | [symbol] | 3.8 | 3.8 | 0.9 | 22.8 | [symbol] | [symbol] | [symbol] | 1.9 | |
| | 12 | 1.9 | [symbol] | 2.9 | 4.8 | 1.9 | 22.5 | [symbol] | [symbol] | [symbol] | [symbol] | 1.9 |
| | 13 | 1.9 | 30.8 | 2.9 | 4.8 | 1.9 | 22.5 | [symbol] | [symbol] | [symbol] | [symbol] | 1.9 |
| | 14 | 2.9 | 32.2 | 2.9 | 4.8 | 2.9 | 24.1 | 1.9 | [symbol] | 1.9 | 4.8 | 2.9 |
| | 15 | 1.9 | 32.2 | 3.8 | 3.8 | 1.9 | 24.1 | 2.9 | [symbol] | 2.9 | 3.8 | [symbol] |
| | 16 | 2.9 | [symbol] | [symbol] | 4.8 | 2.9 | 24.1 | 1.9 | [symbol] | 1.9 | 3.8 | [symbol] |
| | 17 | 0.9 | 30.8 | 3.8 | [symbol] | 0.9 | 22.8 | 1.9 | [symbol] | 1.9 | 2.9 | [symbol] |
| | 18 | 2.9 | [symbol] | 0.9 | [symbol] | 2.9 | 24.1 | 1.9 | 8.9 | 1.9 | 2.9 | [symbol] |
| | 19 | 3.8 | 35.1 | 3.8 | [symbol] | 3.8 | 25.4 | [symbol] | 7.9 | 4.8 | 2.9 | [symbol] |
| | 20 | 3.8 | 33.8 | 3.8 | 5.8 | 3.8 | [symbol] | [symbol] | 6.9 | [symbol] | [symbol] | 2.9 |
| | | 1 | 2 | 3 | 4 | 5 | 6 | 7 | 8 | 9 | 10 | 11 |

| | | Percent Identity | | | | | | | | | | |
|---|---|---|---|---|---|---|---|---|---|---|---|---|
| | | 12 | 13 | 14 | 15 | 16 | 17 | 18 | 19 | 20 | | |
| Percent | 1 | [symbol] | [symbol] | 97.2 | [symbol] | 97.2 | [symbol] | 97.2 | [symbol] | [symbol] | 1 | ADI [symbol] |
| divergence | 2 | 75.7 | [symbol] | [symbol] | [symbol] | 73.8 | 74.8 | 73.8 | 72.0 | [symbol] | 2 | ADI [symbol] |
| | 3 | 97.2 | 97.2 | 97.2 | 96.5 | 97.2 | [symbol] | [symbol] | [symbol] | [symbol] | 3 | ADI [symbol] |
| | 4 | 95.3 | [symbol] | [symbol] | 98.3 | [symbol] | [symbol] | 98.3 | 98.3 | 94.4 | 4 | ADI [symbol] |
| | 5 | 98.1 | [symbol] | [symbol] | [symbol] | [symbol] | 93.1 | 97.2 | 98.3 | 96.3 | 5 | ADI [symbol] |
| | 6 | [symbol] | [symbol] | 79.4 | [symbol] | 79.4 | 80.4 | 79.4 | 78.5 | [symbol] | 6 | ADI [symbol] |
| | 7 | 100.0 | [symbol] | 98.1 | 97.2 | [symbol] | [symbol] | 98.1 | 95.3 | 97.2 | 7 | ADI [symbol] |
| | 8 | [symbol] | 94.4 | [symbol] | [symbol] | [symbol] | [symbol] | 93.5 | [symbol] | [symbol] | 8 | ADI [symbol] |
| | 9 | [symbol] | [symbol] | [symbol] | 97.2 | 98.1 | [symbol] | 98.1 | [symbol] | 97.2 | 9 | ADI [symbol] |
| | 10 | [symbol] | [symbol] | [symbol] | [symbol] | [symbol] | [symbol] | 97.2 | 97.2 | [symbol] | 10 | ADI [symbol] |
| | 11 | [symbol] | [symbol] | 97.2 | [symbol] | 98.1 | [symbol] | 97.2 | 98.3 | 97.2 | 11 | ADI [symbol] |
| | 12 | | [symbol] | 97.2 | 97.2 | 98.1 | [symbol] | [symbol] | [symbol] | 97.2 | 12 | ADI [symbol] |
| | 13 | [symbol] | | 98.1 | 98.1 | 98.1 | [symbol] | [symbol] | [symbol] | 97.2 | 13 | ADI [symbol] |
| | 14 | 1.9 | 1.9 | | 98.1 | 98.1 | [symbol] | 97.2 | 86.3 | 97.2 | 14 | ADI [symbol] |
| | 15 | 2.9 | 2.9 | 1.9 | | 97.2 | [symbol] | 98.3 | 98.1 | [symbol] | 15 | ADI [symbol] |
| | 16 | 1.9 | [symbol] | 1.9 | [symbol] | | [symbol] | 97.2 | [symbol] | [symbol] | 16 | ADI [symbol] |
| | 17 | 1.9 | [symbol] | 2.9 | [symbol] | 2.9 | | 97.2 | [symbol] | [symbol] | 17 | ADI [symbol] |
| | 18 | [symbol] | 1.9 | 2.9 | [symbol] | 2.9 | 2.9 | | 98.3 | [symbol] | 18 | ADI [symbol] |
| | 19 | [symbol] | 4.9 | 3.8 | [symbol] | 4.8 | [symbol] | [symbol] | | [symbol] | 19 | ADI [symbol] |
| | 20 | [symbol] | 2.9 | 2.0 | [symbol] | [symbol] | [symbol] | 3.8 | [symbol] | | 20 | ADI [symbol] |
| | | 12 | 13 | 14 | 15 | 16 | 17 | 18 | 19 | 20 | | |

[symbol]

indicates data missing or illegible when filed

One advantage of the antibody heavy chain variable domain amino acid sequences described above is that they can bind to NKG2D from humans, mice, or cynomolgus monkeys, agonize the receptor, and compete with natural ligands for binding to the receptor. Other antigen-binding sites that bind NKG2D and share one or more of these properties are also particularly useful. Thus, for example, antigen-binding sites that compete with ADI-27705 for binding to both human and mouse NKG2D (and, optionally, cynomolgus monkey NKG2D) are useful. The full length antibody heavy and light chain sequences are listed in the following Table 3:

TABLE 3
ADI-27705 full-length sequences

| Heavy chain amino acid sequence | Light chain amino acid sequence |
|---|---|
| QVQLQQWGAGLLKPSETLSLTCAVY | DIQMTQSPSTLSASVGDRVTITCRASQSISS |
| GGSFSGYYWSWIRQPPGKGLEWIGEI | WLAWYQQKPGKAPKLLIYKASSLESGVPS |
| DHSGSTNYNPSLKSRVTISVDTSKNQF | RFSGSGSGTEFTLTISSLQPDDFATYYCQQ |
| SLKLSSVTAADTAVYYCARARGPWSF | YNSYPITFGGGTKVEIKRTVAAPSVFIFPPS |
| DPWGQGTLVTVSSASTKGPSVFPLAP | DEQLKSGTASVVCLLNNFYPREAKVQWK |
| SSKSTSGGTAALGCLVKDYFPEPVTVS | VDNALQSGNSQESVTEQDSKDSTYSLSST |
| WNSGALTSGVHTFPAVLQSSGLYSLS | LTLSKADYEKHKVYACEVTHQGLSSPVTK |
| SVVTVPSSSLGTQTYICNVNHKPSNTK | SFNRGEC |
| VDKKVEPKSCDKTHTCPPCPAPELLG | (SEQ ID NO: 7) |
| GPSVFLFPPKPKDTLMISRTPEVTCVV | |
| VDVSHEDPEVKFNWYVDGVEVHNAK | |
| TKPREEQYNSTYRVVSVLTVLHQDW | |
| LNGKEYKCKVSNKALPAPIEKTISKAK | |
| GQPREPQVYTLPPSRDELTKNQVSLTC | |
| LVKGFYPSDIAVEWESNGQPENNYKT | |
| TPPVLDSDGSFFLYSKLTVDKSRWQQ | |
| GNVFSCSVMHEALHNHYTQKSLSLSP | |
| GK | |
| (SEQ ID NO: 6) | |

Additional useful antigen-binding sites include those formed by the antibody heavy and light chain pairings listed in the following Table 4:

TABLE 4

| | Heavy chain variable region | Light chain variable |
|---|---|---|
| Clones | amino acid sequence | region amino acid sequence |
| ADI-29443 | QLQLQESGPGLVKPSETLSLTCTVSGG | EIVLTQSPATLSLSPGERATLSCR |
| (F43) | SISSSSYYWGWIRQPPGKGLEWIGSIY | ASQSVSRYLAWYQQKPGQAPR |
| | YSGSTYYNPSLKSRVTISVDTSKNQFS | LLIYDASNRATGIPARFSGSGSG |
| | LKLSSVTAADTAVYYCARGSDRFHPY | TDFTLTISSLEPEDFAVYYCQQF |
| | FDYWGQGTLVTVSS | DTWPPTFGGGTKVEIK |
| | (SEQ ID NO: 33) | (SEQ ID NO: 34) |
| ADI-27727 | QVQLVQSGAEVKKPGSSVKVSCKAS | DIVMTQSPDSLAVSLGERATINC |
| | GGTFSSYAISWVRQAPGQGLEWMGGI | KSSQSVLYSSNNKNYLAWYQQ |
| | IPIFGTANYAQKFQGRVTITADESTST | KPGQPPKLLIYWASTRESGVPDR |
| | AYMELSSLRSEDTAVYYCARGDSSIR | FSGSGSGTDFTLTISSLQAEDVA |
| | HAYYYYGMDVWGQGTTVTVSS | VYYCQQYYSTPITFGGGTKVEIK |
| | (SEQ ID NO: 35) | (SEQ ID NO: 36) |

Similarly, antigen-binding sites that compete with ADI-29443 and/or ADI-27727 for binding to mouse and human NKG2D are useful. The full-length sequences for each antibody are depicted in Table 5, below:

TABLE 5

| | Full-length sequences | |
|---|---|---|
| | Heavy chain amino | Light chain amino |
| Clones | acid sequence | acid sequence |
| ADI-29443 | QLQLQESGPGLVKPSETLSLTCTVSGG | EIVLTQSPATLSLSPGERATLSCR |
| (F43) | SISSSSYYWGWIRQPPGKGLEWIGSIY | ASQSVSRYLAWYQQKPGQAPR |
| | YSGSTYYNPSLKSRVTISVDTSKNQFS | LLIYDASNRATGIPARFSGSGSG |
| | LKLSSVTAADTAVYYCARGSDRFHPY | TDFTLTISSLEPEDFAVYYCQQF |
| | FDYWGQGTLVTVSSASTKGPSVFPLA | DTWPPTFGGGTKVEIKRTVAAP |
| | PSSKSTSGGTAALGCLVKDYFPEPVTV | SVFIFPPSDEQLKSGTASVVCLL |
| | SWNSGALTSGVHTFPAVLQSSGLYSL | NNFYPREAKVQWKVDNALQSG |
| | SSVVTVPSSSLGTQTYICNVNHKPSNT | NSQESVTEQDSKDSTYSLSSTLT |
| | KVDKKVEPKSCDKTHTCPPCPAPELL | LSKADYEKHKVYACEVTHQGL |
| | GGPSVFLFPPKPKDTLMISRTPEVTCV | SSPVTKSFNRGEC |
| | VVDVSHEDPEVKFNWYVDGVEVHNA | (SEQ ID NO: 9) |
| | KTKPREEQYNSTYRVVSVLTVLHQD | |
| | WLNGKEYKCKVSNKALPAPIEKTISK | |
| | AKGQPREPQVYTLPPSRDELTKNQVS | |
| | LTCLVKGFYPSDIAVEWESNGQPENN | |
| | YKTTPPVLDSDGSFFLYSKLTVDKSR | |
| | WQQGNVFSCSVMHEALHNHYTQKSL | |
| | SLSPGK | |
| | (SEQ ID NO: 8) | |
| ADI-27727 | QVQLVQSGAEVKKPGSSVKVSCKAS | DIVMTQSPDSLAVSLGERATINC |
| | GGTFSSYAISWVRQAPGQGLEWMGGI | KSSQSVLYSSNNKNYLAWYQQ |
| | IPIFGTANYAQKFQGRVTITADESTST | KPGQPPKLLIYWASTRESGVPDR |
| | AYMELSSLRSEDTAVYYCARGDSSIR | FSGSGSGTDFTLTISSLQAEDVA |
| | HAYYYYGMDVWGQGTTVTVSSAST | VYYCQQYYSTPITFGGGTKVEIK |
| | KGPSVFPLAPSSKSTSGGTAALGCLVK | RTVAAPSVFIFPPSDEQLKSGTA |
| | DYFPEPVTVSWNSGALTSGVHTFPAV | SVVCLLNNFYPREAKVQWKVD |
| | LQSSGLYSLSSVVTVPSSSLGTQTYIC | NALQSGNSQESVTEQDSKDSTY |
| | NVNHKPSNTKVDKKVEPKSCDKTHT | SLSSTLTLSKADYEKHKVYACE |
| | CPPCPAPELLGGPSVFLFPPKPKDTLMI | VTHQGLSSPVTKSFNRGEC |
| | SRTPEVTCVVVDVSHEDPEVKFNWY | (SEQ ID NO: 11) |
| | VDGVEVHNAKTKPREEQYNSTYRVV | |
| | SVLTVLHQDWLNGKEYKCKVSNKAL | |
| | PAPIEKTISKAKGQPREPQVYTLPPSRD | |
| | ELTKNQVSLTCLVKGFYPSDIAVEWE | |
| | SNGQPENNYKTTPPVLDSDGSFFLYSK | |
| | LTVDKSRWQQGNVFSCSVMHEALHN | |
| | HYTQKSLSLSPGK | |
| | (SEQ ID NO: 10) | |

Assays for binding competition can be performed by methods known in the art, including those described in Example 2.

As but one example described herein in some detail, the antibody heavy chain variable domain amino acid sequences described herein and the antigen-binding sites they can form can be incorporated into larger proteins such as intact antibodies or multi-specific antibodies which can bind to multiple targets. For example, an antigen-binding site that binds NKG2D can be combined with a second component, e.g. a second antigen-binding site, that binds to one or more tumor-associated antigens, such as EpCAM, BCMA, CD33, HER2, CD2, CD3, CD8, CD10, CD19, CD20, CD21, CD22, CD23, CD24, CD25, CD30, CD33, CD37, CD38, CD40, CD45RO, CD48, CD52, CD55, CD59, CD70, CD74, CD80, CD86, CD138, CD147, HLA-DR, CSAp, CA-125, TAG-72, EFGR/ERBB1, IGF1R, HER3, HER4, IGF-1R, c-Met, PDGFR, MUC1, MUC2, MUC3, MUC4, TNFR1, TNFR2, NGFR, TRAILR1, TRAILR2, Fas (CD95), DR3, DR4, DR5, DR6, VEGF, PIGF, tenascin, ED-B fibronectin, PSA, and IL-6, MAGE-A3, B7.1, B7.2, CTLA4 or PD1.

Additional components could also be incorporated, such as a constant domain or a third-antigen binding site that binds to CD16, an Fc receptor on the surface of leukocytes including natural killer cells, macrophages, neutrophils, eosinophils, mast cells, and follicular dendritic cells.

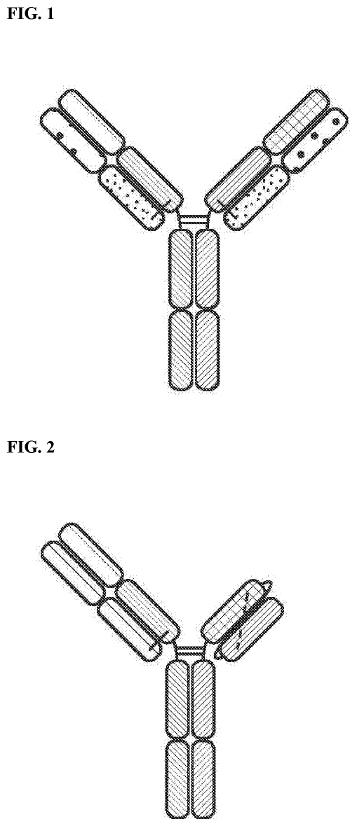

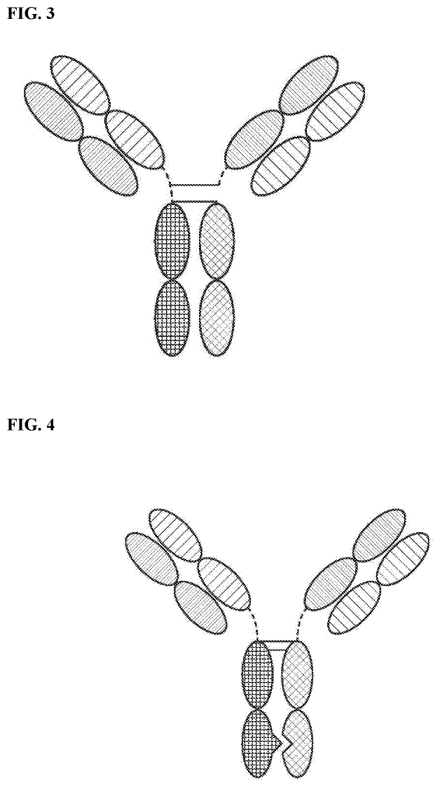

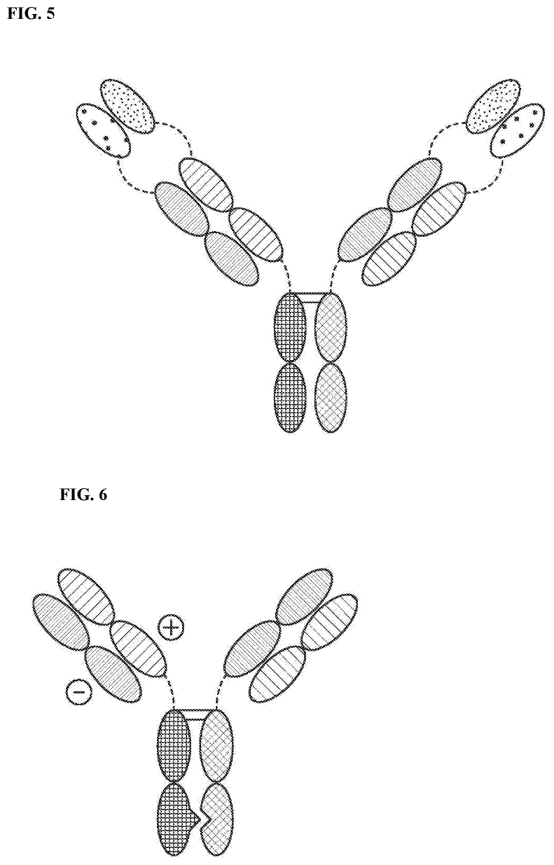

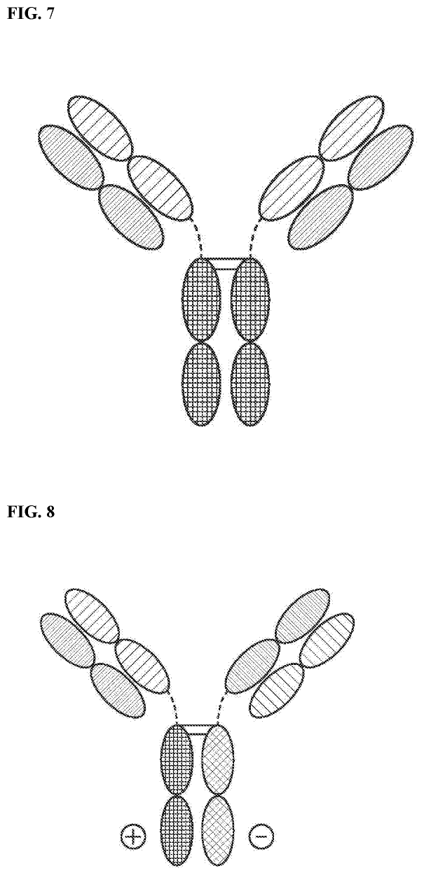

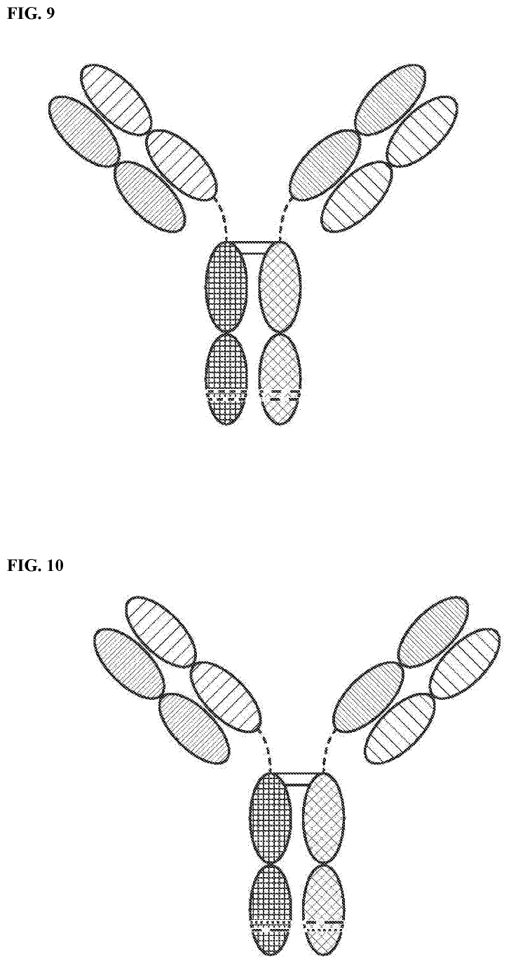

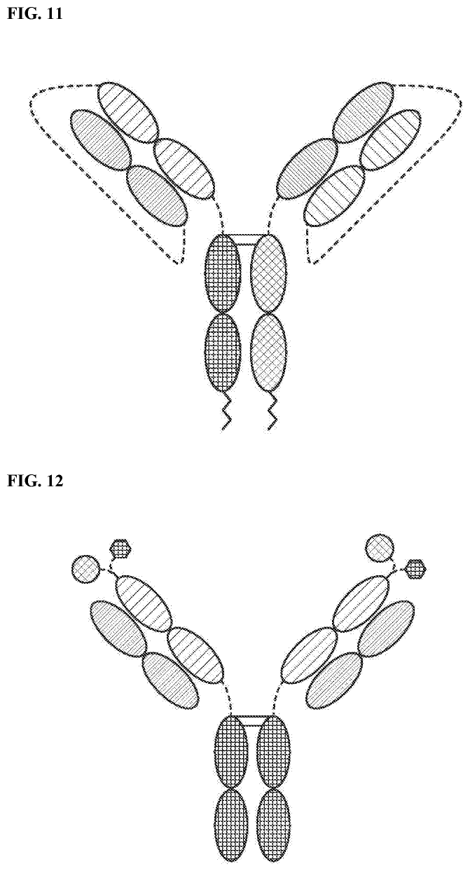

A multi-specific binding protein can take any of several formats. One format is a heterodimeric, multi-specific antibody which includes a first immunoglobulin heavy chain, a second immunoglobulin heavy chain and an immunoglobulin light chain. The first immunoglobulin heavy chain includes a first Fc (hinge-CH2-CH3) domain, a first variable heavy chain domain and a first CH1 heavy chain domain. The immunoglobulin light chain includes a variable light chain domain and a constant light chain domain; together with the first immunoglobulin heavy chain, the immunoglobulin light chain forms an antigen-binding site that binds NKG2D. The second immunoglobulin heavy chain comprises a second Fc (hinge-CH2-CH3) domain, a second variable heavy chain domain and a second CH1 heavy chain domain that may pair with an immunoglobulin light chain identical to the one that pairs with the first immunoglobulin heavy chain, except that when the immunoglobulin light chain is paired with the second immunoglobulin heavy chain, the resulting antigen binding site binds to a tumor antigen. The first Fc domain and second Fc domain together are able to bind to CD16 (FIG. 1).

Within the Fc domain, CD16 binding is mediated by the hinge region and the CH2 domain. For example, within human IgG1, the interaction with CD16 is primarily focused on amino acid residues Asp 265-Glu 269, Asn 297-Thr 299, Ala 327-Ile 332, Leu 234-Ser 239, and carbohydrate residue N-acetyl-D-glucosamine in the CH2 domain (see, Sondermann et al, Nature, 406(6793):267-273). Based on the known domains, mutations can be selected to enhance or reduce the binding affinity to CD16, such as by using phage-displayed libraries or yeast surface-displayed cDNA libraries, or can be designed based on the known three-dimensional structure of the interaction.

The assembly of heterodimeric antibody heavy chains can be accomplished by expressing two different antibody heavy chain sequences in the same cell, which may lead to the assembly of homodimers of each antibody heavy chain as well as assembly of heterodimers. Promoting the preferential assembly of heterodimers can be accomplished by incorporating different mutations in the CH3 domain of each antibody heavy chain constant region as shown in U.S. Ser. Nos. 13/494,870, 16/028,850, 11/533,709, 12/875,015, 13/289,934, 14/773418, 12/811,207, 13/866,756, 14/647,480, 14/830,336. For example, mutations can be made in the CH3 domain based on human IgG1 and incorporating distinct pairs of amino acid substitutions within a first polypeptide and a second polypeptide that allow these two chains to selectively heterodimerize with each other. The positions of amino acid substitutions illustrated below are all numbered according to the EU index as in Kabat.

In one scenario, an amino acid substitution in the first polypeptide replaces the original amino acid with a larger amino acid, selected from arginine (R), phenylalanine (F), tyrosine (Y) or tryptophan (W), and at least one amino acid substitution in the second polypeptide replaces the original amino acid(s) with a smaller amino acid(s), chosen from alanine (A), serine (S), threonine (T), or valine (V), such that the larger amino acid substitution (a protuberance) fits into the surface of the smaller amino acid substitutions (a cavity). For example, one polypeptide can incorporate a T366W substitution, and the other can incorporate three substitutions including T366S, L368A, and Y407V.

An antibody heavy chain variable domain of the invention can optionally be coupled to an amino acid sequence at least 90% identical to an antibody constant region, such as an IgG constant region including hinge, CH2 and CH3 domains with or without CH1 domain. In some embodiments, the amino acid sequence of the constant region is at least 90% identical to a human antibody constant region, such as an human IgG1 constant region, an IgG2 constant region, IgG3 constant region, or IgG4 constant region. In some other embodiments, the amino acid sequence of the constant region is at least 90% identical to an antibody constant region from another mammal, such as rabbit, dog, cat, mouse, or horse. One or more mutations can be incorporated into the constant region as compared to human IgG1 constant region, for example at Q347, Y349, L351, S354, E356, E357, K360, Q362, S364, T366, L368, K370, N390, K392, T394, D399, S400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, Y407A, Y407I, Y407V, K409F, K409W, K409D, T411D, T411E, K439D, and K439E.

In certain embodiments, mutations that can be incorporated into the CH1 of a human IgG1 constant region may be at amino acid V125, F126, P127, T135, T139, A140, F170, P171, and/or V173. In certain embodiments, mutations that can be incorporated into the Cκ of a human IgG1 constant region may be at amino acid E123, F116, S176, V163, S174, and/or T164.

Alternatively, amino acid substitutions could be selected from the following sets of substitutions shown in Table 6.

TABLE 6

| | First Polypeptide | Second Polypeptide |
|---|---|---|
| Set 1 | S364E/F405A | Y349K/T394F |
| Set 2 | S364H/D401K | Y349T/T411E |
| Set 3 | S364H/T394F | Y349T/F405A |
| Set 4 | S364E/T394F | Y349K/F405A |
| Set 5 | S364E/T411E | Y349K/D401K |
| Set 6 | S364D/T394F | Y349K/F405A |
| Set 7 | S364H/F405A | Y349T/T394F |
| Set 8 | S364K/E357Q | L368D/K370S |
| Set 9 | L368D/K370S | S364K |
| Set 10 | L368E/K370S | S364K |
| Set 11 | K360E/Q362E | D401K |
| Set 12 | L368D/K370S | S364K/E357L |
| Set 13 | K370S | S364K/E357Q |
| Set 14 | F405L | K409R |
| Set 15 | K409R | F405L |

Alternatively, amino acid substitutions could be selected from the following sets of substitutions shown in Table 7.

TABLE 7

| | First Polypeptide | Second Polypeptide |
|---|---|---|
| Set 1 | K409W | D399V/F405T |
| Set 2 | Y349S | E357W |
| Set 3 | K360E | Q347R |
| Set 4 | K360E/K409W | Q347R/D399V/F405T |
| Set 5 | Q347E/K360E/K409W | Q347R/D399V/F405T |
| Set 6 | Y349S/K409W | E357W/D399V/F405T |

Alternatively, amino acid substitutions could be selected from the following set of substitutions shown in Table 8.

TABLE 8

| | First Polypeptide | Second Polypeptide |
|---|---|---|
| Set 1 | T366K/L351K | L351D/L368E |
| Set 2 | T366K/L351K | L351D/Y349E |
| Set 3 | T366K/L351K | L351D/Y349D |
| Set 4 | T366K/L351K | L351D/Y349E/L368E |
| Set 5 | T366K/L351K | L351D/Y349D/L368E |
| Set 6 | E356K/D399K | K392D/K409D |

Alternatively, at least one amino acid substitution in each polypeptide chain could be selected from Table 9.

TABLE 9

| First Polypeptide | Second Polypeptide |
|---|---|
| L351Y, D399R, D399K, | T366V, T366I, T366L, T366M, N390D, |
| S400K, S400R, Y407A, | N390E, K392L, K392M, K392V, K392F |
| Y407I, Y407V | K392D, K392E, K409F, K409W, T411D and |
| | T411E |

Alternatively, at least one amino acid substitutions could be selected from the following set of substitutions in Table 10, where the position(s) indicated in the First Polypeptide column is replaced by any known negatively-charged amino acid, and the position(s) indicated in the Second Polypeptide Column is replaced by any known positively-charged amino acid.

TABLE 10

| First Polypeptide | Second Polypeptide |
|---|---|
| K392, K370, K409, or K439 | D399, E356, or E357 |

Alternatively, at least one amino acid substitutions could be selected from the following set of in Table 11, where the position(s) indicated in the First Polypeptide column is replaced by any known positively-charged amino acid, and the position(s) indicated in the Second Polypeptide Column is replaced by any known negatively-charged amino acid.

TABLE 11

| First Polypeptide | Second Polypeptide |
|---|---|
| D399, E356, or E357 | K409, K439, K370, or K392 |

Alternatively, amino acid substitutions could be selected from the following set of in Table 12.

TABLE 12

| First Polypeptide | Second Polypeptide |
|---|---|
| T350V, L351Y, F405A, and | T350V, T366L, K392L, and T394W |
| Y407V | |

Alternatively, or in addition, the structural stability of a heteromultimer protein may be increased by introducing S354C on either of the first or second polypeptide chain, and Y349C on the opposing polypeptide chain, which forms an artificial disulfide bridge within the interface of the two polypeptides.

The multispecific proteins described above can be made using recombinant DNA technology well known to a skilled person in the art. For example, a first nucleic acid sequence encoding the first immunoglobulin heavy chain can be cloned into a first expression vector; a second nucleic acid sequence encoding the second immunoglobulin heavy chain can be cloned into a second expression vector; a third nucleic acid sequence encoding the immunoglobulin light chain can be cloned into a third expression vector; the first, second, and third expression vectors can be stably transfected together into host cells to produce the multimeric proteins.

To achieve the highest yield of the multi-specific protein, different ratios of the first, second, and third expression vector can be explored to determine the optimal ratio for transfection into the host cells. After transfection, single clones can be isolated for cell bank generation using methods known in the art, such as limited dilution, ELISA, FACS, microscopy, or Clonepix.

Clones can be cultured under conditions suitable for bio-reactor scale-up and maintained expression of the multi-specific protein. The multispecific proteins can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

### II. Proteins

The invention provides multi-specific binding proteins that bind a tumor-associated antigen on a cancer cell and the NKG2D receptor and CD16 receptor on natural killer cells to activate the natural killer cell. The multi-specific binding proteins are useful in the pharmaceutical compositions and therapeutic methods described herein. Binding of the multi-specific binding protein to the NKG2D receptor and CD16 receptor on natural killer cell enhances the activity of the natural killer cell toward destruction of a cancer cell. Binding of the multi-specific binding protein to a tumor-associated antigen on a cancer cell brings the cancer cell into proximity to the natural killer cell, which facilitates direct and indirect destruction of the cancer cell by the natural killer cell. Further description of exemplary multi-specific binding proteins are provided below.

The first component of the multi-specific binding proteins binds to NKG2D receptor-expressing cells, which can include but are not limited to NK cells, γδT cells and CD8⁺αβ T cells. Upon NKG2D-binding, the multi-specific binding proteins may block natural ligands, such as ULBP6 and MICA, from binding to NKG2D.

The second component of the multi-specific binding proteins binds to one or more tumor-associated antigens, which can include, but are not limited to HER2, CD20, CD33, BCMA, EpCAM, CD2, CD19, CD30, CD38, CD40, CD52, CD70, EGFR/ERBB1, IGF1R, HER2, HER3/ERBB3, HER4/ERBB4, MUC1, cMET, SLAMF7, PSCA, MICA, MICB, TRAILR1, TRAILR2, MAGE-A3, B7.1, B7.2, CTLA4, and PD1.

The third component for the multi-specific binding proteins binds to cells expressing CD16, an Fc receptor on the surface of leukocytes including natural killer cells, macrophages, neutrophils, eosinophils, mast cells, and follicular dendritic cells.

The multi-specific binding proteins can take several formats as shown in but not limited to the examples below. One format is a heterodimeric, multi-specific antibody that includes a first immunoglobulin heavy chain, a second immunoglobulin heavy chain and an immunoglobulin light chain. The first immunoglobulin heavy chain includes a first Fc (hinge-CH2-CH3) domain, a first variable heavy chain domain and an optional first CH1 heavy chain domain. The immunoglobulin light chain includes a variable light chain domain and a constant light chain domain; together with the first immunoglobulin heavy chain, the immunoglobulin light chain forms an antigen-binding site that binds NKG2D. The second immunoglobulin heavy chain comprises a second Fc (hinge-CH2-CH3) domain, a second variable heavy chain domain and a second optional CH1 heavy chain domain that may pair with an immunoglobulin light chain identical to the one that pairs with the first immunoglobulin heavy chain, except that when immunoglobulin light chain is paired with the second immunoglobulin heavy chain, the resulting antigen binding site binds to a tumor antigen. The first Fc domain and second Fc domain together are able to bind to CD16 (FIG. 1).

Another exemplary format involves a heterodimeric, multi-specific antibody which includes a first immunoglobulin heavy chain, an immunoglobulin light chain and a second immunoglobulin heavy chain. The first immunoglobulin heavy chain includes a first Fc (hinge-CH2-CH3) domain fused via either a linker or an antibody hinge to a single chain Fv (scFv) that binds NKG2D. A variety of linkers could be used for linking the scFv to the first Fc domain or within the scFv itself. In addition, the scFv can incorporate mutations that enable the formation of a disulfide bond to stabilize the overall scFv structure. The scFv can also incorporate mutations to modify the isoelectric point of the overall first immunoglobulin heavy chain and/or to enable more facile downstream purification. The second immunoglobulin heavy chain includes a second Fc (hinge-CH2-CH3) domain and a second variable heavy chain domain and a second optional CH1 heavy chain domain. The immunoglobulin light chain includes a variable light chain domain and a constant light chain domain. The second immunoglobulin heavy chain pairs with the immunoglobulin light chain and binds to a tumor antigen. The first Fc domain and the second Fc domain together are able to bind to CD16 (FIG. 2).

An alternative format of the heterodimeric multi-specific proteins includes a first immunoglobulin heavy chain, a second immunoglobulin heavy chain, a first immunoglobulin light chain and a second immunoglobulin light chain. The first immunoglobulin heavy chain includes a first Fc (hinge-CH2-CH3) domain, a first variable heavy chain domain and an optional first CH1 heavy chain domain. The first immunoglobulin light chain includes a variable light chain domain and a constant light chain domain. Together with the first immunoglobulin heavy chain, the first immunoglobulin light chain forms an antigen-binding site that binds a tumor antigen. The second immunoglobulin heavy chain comprises a second Fc (hinge-CH2-CH3) domain, a second variable heavy chain domain and a second optional CH1 heavy chain domain. The second immunoglobulin light chain includes a variable light chain domain and a constant light chain domain. Together with the second immunoglobulin heavy chain, the immunoglobulin light chain forms an antigen-binding site that binds to the same tumor antigen. The second immunoglobulin heavy chain may pair with an immunoglobulin light chain, which may be identical to the immunoglobulin light chain that pairs with the first immunoglobulin heavy chain, except that when immunoglobulin light chain is paired with the second immunoglobulin heavy chain, the resulting antigen binding site is a second antigen-binding site that binds to a tumor antigen. In certain embodiments, the first Fc domain and second Fc domain together are able to bind to CD16 (FIG. 1).

One or more additional binding motifs may be fused to the C-terminus of the constant region CH3 domain, optionally via a linker sequence. In certain embodiments, the antigen-binding site could be a single-chain or disulfide-stabilized variable region (ScFv) or could form a tetravalent or trivalent molecule.

In some embodiments, the multi-specific binding protein is in the Triomab form, which is a trifunctional, bispecific antibody that maintains an IgG-like shape. This chimera consists of two half antibodies, each with one light and one heavy chain, that originate from two parental antibodies.

In some embodiments, the multi-specific binding protein is the KiH Common Light Chain (LC) form, which involves the knobs-into-holes (KIHs) technology. The KIH involves engineering C_{H}3 domains to create either a “knob” or a “hole” in each heavy chain to promote heterodimerization. The concept behind the “Knobs-into-Holes (KiH)” Fc technology was to introduce a “knob” in one CH3 domain (CH3A) by substitution of a small residue with a bulky one (i.e., T366W_{CH3A} in EU numbering). To accommodate the “knob,” a complementary “hole” surface was created on the other CH3 domain (CH3B) by replacing the closest neighboring residues to the knob with smaller ones (i.e., T366S/L368A/Y407V_{CH3B}). The “hole” mutation was optimized by structured-guided phage library screening (Atwell S, Ridgway J B, Wells J A, Carter P. Stable heterodimers from remodeling the domain interface of a homodimer using a phage display library. J Mol Biol (1997) 270(1):26-35). X-ray crystal structures of KiH Fc variants (Elliott J M, Ultsch M, Lee J, Tong R, Takeda K, Spiess C, et al., Antiparallel conformation of knob and hole aglycosylated half-antibody homodimers is mediated by a CH2-CH3 hydrophobic interaction. J Mol Biol (2014) 426(9):1947-57; Mimoto F, Kadono S, Katada H, Igawa T, Kamikawa T, Hattori K. Crystal structure of a novel asymmetrically engineered Fc variant with improved affinity for FcgammaRs Mol Immunol (2014) 58(1):132-8) demonstrated that heterodimerization is thermodynamically favored by hydrophobic interactions driven by steric complementarity at the inter-CH3 domain core interface, whereas the knob-knob and the hole-hole interfaces do not favor homodimerization owing to steric hindrance and disruption of the favorable interactions, respectively.

In some embodiments, the multi-specific binding protein is in the dual-variable domain immunoglobulin (DVD-Ig™) form, which combines the target binding domains of two monoclonal antibodies via flexible naturally occurring linkers, and yields a tetravalent IgG—like molecule.

In some embodiments, the multi-specific binding protein is in the Orthogonal Fab interface (Ortho-Fab) form. In ortho-Fab IgG approach (Lewis S M, Wu X, Pustilnik A, Sereno A, Huang F, Rick H L, et al. Generation of bispecific IgG antibodies by structure-based design of an orthogonal Fab interface. Nat. Biotechnol. (2014) 32(2):191-8), structure-based regional design introduces complementary mutations at the LC and HC_{VU-CH1} interface in only one Fab, without any changes being made to the other Fab.

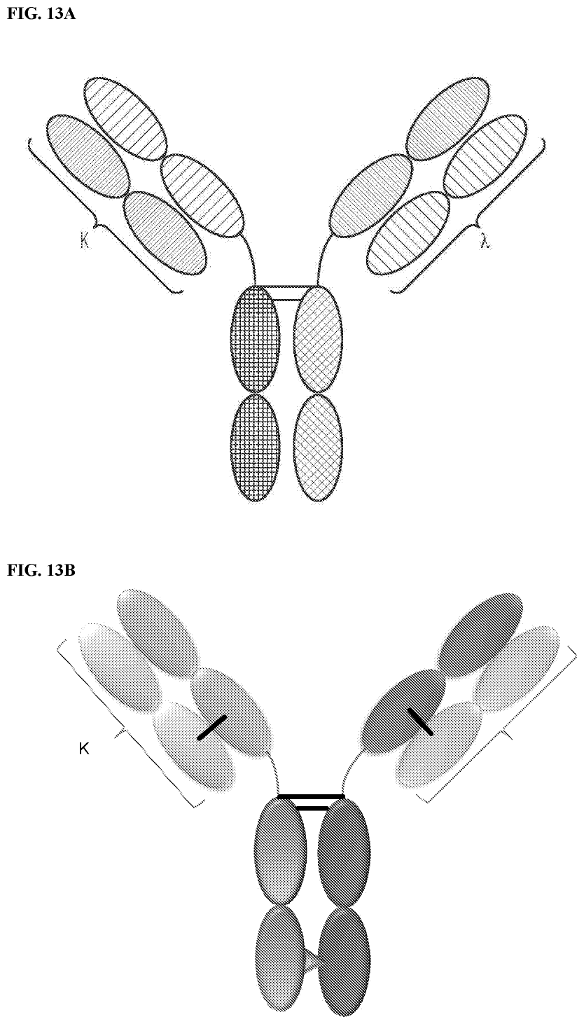

In some embodiments, the multi-specific binding protein is in the 2 in 1Ig format. In some embodiments, the multi-specific binding protein is in the ES form, which is an heterodimeric construct containing 2 different Fabs binding to target 1 and target 2 fused to the F_{C}. Heterodimerization is ensured by electrostatic steering mutations in the Fc. In some embodiments, the multi-specific binding protein is in the κλ-Body form, which is an heterodimeric constructs with 2 different Fabs fused to Fc stabilized by heterodimerization mutations: Fab1 targeting antigen 1 contains kappa LC, while second Fab targeting antigen 2 contains lambda LC. FIG. 13A is an exemplary representation of one form of a κλ-Body; FIG. 13B is an exemplary representation of another κλ-Body.

In some embodiments, the multi-specific binding protein is in Fab Arm Exchange form (antibodies that exchange Fab arms by swapping a heavy chain and attached light chain (half-molecule) with a heavy-light chain pair from another molecule, which results in bispecific antibodies). In some embodiments, the multi-specific binding protein is in the SEED Body form (The strand-exchange engineered domain (SEED) platform was designed to generate asymmetric and bispecific antibody-like molecules, a capability that expands therapeutic applications of natural antibodies. This protein engineered platform is based on exchanging structurally related sequences of immunoglobulin within the conserved CH3 domains. The SEED design allows efficient generation of AG/GA heterodimers, while disfavoring homodimerization of AG and GA SEED CH3 domains. (Muda M. et al., Protein Eng. Des. Sel. (2011, 24(5):447-54)). In some embodiments, the multi-specific binding protein is in the LuZ-Y form, in which leucine zipper is used to induce heterodimerization of two different HCs. (Wranik, B J. et al., J. Biol. Chem. (2012), 287:43331-9).

In some embodiments, the multi-specific binding protein is in the Cov-X-Body form (In bispecific CovX-Bodies, two different peptides are joined together using a branched azetidinone linker and fused to the scaffold antibody under mild conditions in a site-specific manner. Whereas the pharmacophores are responsible for functional activities, the antibody scaffold imparts long half-life and Ig-like distribution. The pharmacophores can be chemically optimized or replaced with other pharmacophores to generate optimized or unique bispecific antibodies. (Doppalapudi V R et al., PNAS (2010), 107(52); —22611-22616).

### III. Characteristics of TriNKETs

In certain embodiments, TriNKETs described herein, which include an NKG2D-binding domain and a binding domain for a tumor associated antigen, bind to cells expressing human NKG2D. In certain embodiments, TriNKETs, which include an NKG2D-binding domain and a binding domain for a tumor associated antigen, bind to the tumor associated antigen at a comparable level to that of a monoclonal antibody having the same tumor associated antigen-binding domain. For example, TriNKETs that include an NKG2D-binding domain and a HER2-binding domain from Trastuzumab can bind to HER2 expressed on cells at a level comparable to that of Trastuzumab.

However, the TriNKETs described herein are more effective in reducing tumor growth and killing cancer cells. For example, a TriNKET of the present disclosure that targets HER2-expressing tumor/cancer cells is more effective than SC2.2—a single chain bispecific molecule built from an scFv derived from trastuzumab linked to ULBP-6, a ligand for NKG2D. SC2.2 binds HER2+ cancer cells and NKG2D+ NK cells simultaneously. Therefore, effectiveness of SC2.2 in reducing HER2+ cancer cell number was investigated. In vitro activation and cytotoxity assays demonstrated that SC2.2 was effective in activating and killing NK cells. However, SC2.2 failed to demonstrate efficacy in the RMA/S-HER2 subcutaneous tumor model. The efficacy of SC2.2 was also tested in vivo using an RMA/S-HER2 overexpressing syngeneic mouse model. In this mouse model, SC2.2 failed to demonstrate control of tumor growth compared to vehicle control. Thus, although SC2.2 was able to activate and kill NK cells, and binds to HER2+ cancer cells, these properties were insufficient to effectively control HER2+ tumor growth.

In certain embodiments, TriNKETs described herein, which include an NKG2D-binding domain and a binding domain for tumor associated antigen, activate primary human NK cells when culturing with tumor cells expressing the antigen. NK cell activation is marked by the increase in CD107a degranulation and IFNγ cytokine production. Furthermore, compared to a monoclonal antibody that includes the tumor associated antigen-binding domain, TriNKETs show superior activation of human NK cells in the presence of tumor cells expressing the antigen. For example, compared to the monoclonal antibody trastuzumab, TriNKETs of the present disclosure having a HER2-binding domain, have a superior activation of human NK cells in the presence of HER2-expressing cancer cells.

In certain embodiments, TriNKETs described herein, which include an NKG2D-binding domain and a binding domain for a tumor associated antigen, enhance the activity of rested and IL-2-activated human NK cells in the presence of tumor cells expressing the antigen. Rested NK cells showed less background IFNγ production and CD107a degranulation than IL-2-activated NK cells. In certain embodiments, rested NK cells show a greater change in IFNγ production and CD107a degranulation compared to IL-2-activated NK cells. In certain embodiments, IL-2-activated NK cells show a greater percentage of cells becoming IFNγ+; CD107a+ after stimulation with TriNKETs.

In certain embodiments, TriNKETs described herein, which include an NKG2D-binding domain and a binding domain for a tumor associated antigen (non-limiting examples of tumor associated antigens including CD20, BCMA, and HER2), enhance the cytotoxic activity of rested and IL-2-activated human NK cells in the presence of tumor cells expressing the antigen. Furthermore, TriNKETs (e.g., A40-TriNKET, C26-TriNKET, F04-TriNKET, F43-TriNKET, and F47-TriNKET), which include a binding domain for a tumor associated antigen (non-limiting examples of tumor associated antigens including CD20, BCMA, and HER2) more potently direct activated and rested NK cell responses against the tumor cells, compared to a monoclonal antibody that includes the same tumor associated antigen binding site. In certain embodiments, TriNKETs offer advantage against tumor cells expressing medium and low tumor antigens compared to monoclonal antibodies that include the same tumor antigen binding site. Therefore, a therapy including TriNKETs can be superior to a monoclonal antibody therapy.

In certain embodiments, compared to monoclonal antibodies, TriNKETs described herein (e.g., A40-TriNKET, C26-TriNKET, F04-TriNKET, F43-TriNKET, and F47-TriNKET), which include a binding domain for a tumor associated antigen (non-limiting examples of tumor associated antigens including CD20, BCMA, and HER2) are advantageous in treating cancers with high expression of Fc receptor (FcR), or cancers residing in a tumor microenvironment with high levels of FcR. Monoclonal antibodies exert their effects on tumor growth through multiple mechanisms including ADCC, CDC, phagocytosis, and signal blockade amongst others. Amongst FcγRs, CD16 has the lowest affinity for IgG Fc; FcγRI (CD64) is the high-affinity FcR, which binds about 1000 times more strongly to IgG Fc than CD16. CD64 is normally expressed on many hematopoietic lineages such as the myeloid lineage, and can be expressed on tumors derived from these cell types, such as acute myeloid leukemia (AML) Immune cells infiltrating into the tumor, such as MDSCs and monocytes, also express CD64 and are known to infiltrate the tumor microenvironment. Expression of CD64 by the tumor or in the tumor microenvironment can have a detrimental effect on monoclonal antibody therapy. Expression of CD64 in the tumor microenvironment makes it difficult for these antibodies to engage CD16 on the surface of NK cells, as the antibodies prefer to bind the high-affinity receptor. TriNKETs, through targeting two activating receptors on the surface of NK cells, can overcome the detrimental effect of CD64 expression (either on tumor or tumor microenvironment) on monoclonal antibody therapy. Regardless of CD64 expression on the tumor cells, TriNKETs are able to mediate human NK cell responses against all tumor cells, because dual targeting of two activating receptors on NK cells provides stronger specific binding to NK cells.

In some embodiments, TriNKETs described herein (e.g., A40-TriNKET, C26-TriNKET, F04-TriNKET, F43-TriNKET, and F47-TriNKET), which include a binding domain for a tumor associated antigen (non-limiting examples of tumor associated antigens including CD20, BCMA, and HER2) provide a better safety profile through reduced on-target off-tumor side effects. Natural killer cells and CD8 T cells are both able to directly lyse tumor cells, although the mechanisms through which NK cells and CD8 T cell recognize normal self from tumor cells differ. The activity of NK cells is regulated by the balance of signals from activating (NCRs, NKG2D, CD16, etc.) and inhibitory (KIRs, NKG2A, etc.) receptors. The balance of these activating and inhibitory signals allow NK cells to determine healthy self-cells from stressed, virally infected, or transformed self-cells. This ‘built-in’ mechanism of self-tolerance will help protect normal healthy tissue from NK cell responses. To extend this principle, the self-tolerance of NK cells will allow TriNKETs to target antigens expressed both on self and tumor without off tumor side effects, or with an increased therapeutic window. Unlike natural killer cells, T cells require recognition of a specific peptide presented by MHC molecules for activation and effector functions. T cells have been the primary target of immunotherapy, and many strategies have been developed to redirect T cell responses against the tumor. T cell bispecifics, checkpoint inhibitors, and CAR-T cells have all been approved by the FDA, but often suffer from dose-limiting toxicities. T cell bispecifics and CAR-T cells work around the TCR-MHC recognition system by using binding domains to target antigens on the surface of tumor cells, and using engineered signaling domains to transduce the activation signals into the effector cell. Although effective at eliciting an anti-tumor immune response these therapies are often coupled with cytokine release syndrome (CRS), and on-target off-tumor side effects. TriNKETs are unique in this context as they will not ‘override’ the natural systems of NK cell activation and inhibition. Instead, TriNKETs are designed to sway the balance, and provide additional activation signals to the NK cells, while maintaining NK tolerance to healthy self.

In some embodiments, TriNKETs described herein including an NKG2D-binding domain (e.g., A40-TriNKET, C26-TriNKET, F04-TriNKET, F43-TriNKET, and F47-TriNKET), which include a binding domain for a tumor associated antigen (non-limiting examples of tumor associated antigens including CD20, BCMA, and HER2) delay progression of the tumor more effectively than monoclonal antibodies that include the same tumor antigen-binding domain. In some embodiments, TriNKETs including an NKG2D-binding domain and a tumor antigen-binding domain are more effective against cancer metastases than monoclonal antibodies that include the same tumor antigen-binding domain.

The description above describes multiple aspects and embodiments of the invention. The patent application specifically contemplates all combinations and permutations of the aspects and embodiments.

### IV. Therapeutic Applications

The invention provides methods for treating cancer using a protein described herein and/or a pharmaceutical composition described herein. The methods may be used to treat a variety of cancers, including a solid tumor, a lymphoma, and a leukemia. The type of cancer to be treated is desirably matched with the type of cancer cell to which the protein binds. For example, treatment of a cancer expressing epithelial cell adhesion molecule (EpCAM), such as a colon cancer expressing EpCAM, is desirably treated using a protein described herein that binds to protein. Additional aspects and embodiments of the therapeutic methods are described below.

Accordingly, one aspect of the invention provides a method of treating cancer in a patient, wherein the method comprises administering to a patient in need thereof a therapeutically effective amount of a protein described herein to treat the cancer. Exemplary cancers for treatment include a solid tumor, leukemia, and lymphoma.

The therapeutic method can be characterized according to the cancer to be treated. For example, in certain embodiments, the cancer is a solid tumor. In certain other embodiments, the cancer is brain cancer, bladder cancer, breast cancer, cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, or uterine cancer. In yet other embodiments, the cancer is a vascularized tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, bilary tract cancer, thyroid cancer, acral lentiginous melanoma, actinic keratoses, acute lymphocytic leukemia, acute myeloid leukemia, adenoid cycstic carcinoma, adenomas, adenosarcoma, adenosquamous carcinoma, anal canal cancer, anal cancer, anorectum cancer, astrocytic tumor, bartholin gland carcinoma, basal cell carcinoma, biliary cancer, bone cancer, bone marrow cancer, bronchial cancer, bronchial gland carcinoma, carcinoid, cholangiocarcinoma, chondosarcoma, choriod plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intaepithelial neoplasia, interepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VlPoma, vulva cancer, well differentiated carcinoma, or Wilms tumor.

In certain other embodiments, the cancer is non-Hodgkin's lymphoma, such as a B-cell lymphoma or a T-cell lymphoma. In certain embodiments, the non-Hodgkin's lymphoma is a B-cell lymphoma, such as a diffuse large B-cell lymphoma, primary mediastinal B-cell lymphoma, follicular lymphoma, small lymphocytic lymphoma, mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, hairy cell leukemia, or primary central nervous system (CNS) lymphoma. In certain other embodiments, the non-Hodgkin's lymphoma is a T-cell lymphoma, such as a precursor T-lymphoblastic lymphoma, peripheral T-cell lymphoma, cutaneous T-cell lymphoma, angioimmunoblastic T-cell lymphoma, extranodal natural killer/T-cell lymphoma, enteropathy type T-cell lymphoma, subcutaneous panniculitis-like T-cell lymphoma, anaplastic large cell lymphoma, or peripheral T-cell lymphoma.

The cancer to be treated can be characterized according to the presence of a particular antigen expressed on the surface of the cancer cell. In certain embodiments, the cancer cell expresses one or more of the following: BCMA, CD33, HER2, CD2, CD19, CD20, CD30, CD38, CD40, CD52, CD70, EGFR/ERBB1, IGF1R, HER3/ERBB3, HER4/ERBB4, MUC1, CEA, cMET, SLAMF7, PSCA, MICA, MICB, TRAILR1, TRAILR2, MAGE-A3, B7.1, B7.2, CTLA4, and PD1.

### V. Combination Therapy

Another aspect of the invention provides for combination therapy. Proteins described herein be used in combination with additional therapeutic agents to treat the cancer.

Exemplary therapeutic agents that may be used as part of a combination therapy in treating cancer, include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, luteinizing hormone releasing factor and variations of the aforementioned agents that may exhibit differential binding to its cognate receptor, and increased or decreased serum half-life.

An additional class of agents that may be used as part of a combination therapy in treating cancer is immune checkpoint inhibitors. Exemplary immune checkpoint inhibitors include agents that inhibit one or more of (i) cytotoxic T-lymphocyte-associated antigen 4 (CTLA4), (ii) programmed cell death protein 1 (PD1), (iii) PDL1, (iv) LAGS, (v) B7-H3, (vi) B7-H4, and (vii) TIM3. The CTLA4 inhibitor ipilimumab has been approved by the United States Food and Drug Administration for treating melanoma.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Yet other categories of anti-cancer agents include, for example: (i) an inhibitor selected from an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor; (ii) an agonist of OX40, CD137, CD40, GITR, CD27, HVEM, TNFRSF25, or ICOS; and (iii) a cytokine selected from IL-12, IL-15, GM-CSF, and G-CSF.

Proteins of the invention can also be used as an adjunct to surgical removal of the primary lesion.

The amount of protein and additional therapeutic agent and the relative timing of administration may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. Further, for example, a protein described herein may be administered during a time when the additional therapeutic agent(s) exerts its prophylactic or therapeutic effect, or vice versa.

### VI. Pharmaceutical Compositions

The present disclosure also features pharmaceutical compositions that contain a therapeutically effective amount of a protein described herein. The composition can be formulated for use in a variety of drug delivery systems. One or more physiologically acceptable excipients or carriers can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

When the proteins of the present invention are administered as pharmaceuticals, to veterinary animals, they can be given as a pharmaceutical composition containing, for example, 0.01 to 20% (more preferably, 0.01 to 0.5%) of active ingredient in combination with a pharmaceutically acceptable carrier.

The intravenous drug delivery formulation of the present disclosure may be contained in a bag, a pen, or a syringe. In certain embodiments, the bag may be connected to a channel comprising a tube and/or a needle. In certain embodiments, the formulation may be a lyophilized formulation or a liquid formulation. In certain embodiments, the formulation may freeze-dried (lyophilized) and contained in about 12-60 vials. In certain embodiments, the formulation may be freeze-dried and 45 mg of the freeze-dried formulation may be contained in one vial. In certain embodiments, the about 40 mg-about 100 mg of freeze-dried formulation may be contained in one vial. In certain embodiments, freeze dried formulation from 12, 27, or 45 vials are combined to obtained a therapeutic dose of the protein in the intravenous drug formulation. In certain embodiments, the formulation may be a liquid formulation and stored as about 250 mg/vial to about 1000 mg/vial. In certain embodiments, the formulation may be a liquid formulation and stored as about 600 mg/vial. In certain embodiments, the formulation may be a liquid formulation and stored as about 250 mg/vial.

This present disclosure could exist in a liquid aqueous pharmaceutical formulation including a therapeutically effective amount of the protein in a buffered solution forming a formulation.

These compositions may be sterilized by conventional sterilization techniques, or may be sterile filtered. The resulting aqueous solutions may be packaged for use as-is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents. The composition in solid form can also be packaged in a container for a flexible quantity.

In certain embodiments, the present disclosure provides a formulation with an extended shelf life including the protein of the present disclosure, in combination with mannitol, citric acid monohydrate, sodium citrate, disodium phosphate dihydrate, sodium dihydrogen phosphate dihydrate, sodium chloride, polysorbate 80, water, and sodium hydroxide.

In certain embodiments, an aqueous formulation is prepared including the protein of the present disclosure in a pH-buffered solution. The buffer of this invention may have a pH ranging from about 4 to about 8, e.g., from about 4.5 to about 6.0, or from about 4.8 to about 5.5, or may have a pH of about 5.0 to about 5.2. Ranges intermediate to the above recited pH's are also intended to be part of this disclosure. For example, ranges of values using a combination of any of the above recited values as upper and/or lower limits are intended to be included. Examples of buffers that will control the pH within this range include acetate (e.g. sodium acetate), succinate (such as sodium succinate), gluconate, histidine, citrate and other organic acid buffers.

In certain embodiments, the formulation includes a buffer system which contains citrate and phosphate to maintain the pH in a range of about 4 to about 8. In certain embodiments the pH range may be from about 4.5 to about 6.0, or from about pH 4.8 to about 5.5, or in a pH range of about 5.0 to about 5.2. In certain embodiments, the buffer system includes citric acid monohydrate, sodium citrate, disodium phosphate dihydrate, and/or sodium dihydrogen phosphate dihydrate. In certain embodiments, the buffer system includes about 1.3 mg/ml of citric acid (e.g., 1.305 mg/ml), about 0.3 mg/ml of sodium citrate (e.g., 0.305 mg/ml), about 1.5 mg/ml of disodium phosphate dihydrate (e.g. 1.53 mg/ml), about 0.9 mg/ml of sodium dihydrogen phosphate dihydrate (e.g., 0.86), and about 6.2 mg/ml of sodium chloride (e.g., 6.165 mg/ml). In certain embodiments, the buffer system includes 1-1.5 mg/ml of citric acid, 0.25 to 0.5 mg/ml of sodium citrate, 1.25 to 1.75 mg/ml of disodium phosphate dihydrate, 0.7 to 1.1 mg/ml of sodium dihydrogen phosphate dihydrate, and 6.0 to 6.4 mg/ml of sodium chloride. In certain embodiments, the pH of the formulation is adjusted with sodium hydroxide.

A polyol, which acts as a tonicifier and may stabilize the antibody, may also be included in the formulation. The polyol is added to the formulation in an amount which may vary with respect to the desired isotonicity of the formulation. In certain embodiments, the aqueous formulation may be isotonic. The amount of polyol added may also be altered with respect to the molecular weight of the polyol. For example, a lower amount of a monosaccharide (e g mannitol) may be added, compared to a disaccharide (such as trehalose). In certain embodiments, the polyol which may be used in the formulation as a tonicity agent is mannitol. In certain embodiments, the mannitol concentration may be about 5 to about 20 mg/ml. In certain embodiments, the concentration of mannitol may be about 7.5 to 15 mg/ml. In certain embodiments, the concentration of mannitol may be about 10-14 mg/ml. In certain embodiments, the concentration of mannitol may be about 12 mg/ml. In certain embodiments, the polyol sorbitol may be included in the formulation.

A detergent or surfactant may also be added to the formulation. Exemplary detergents include nonionic detergents such as polysorbates (e.g. polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In certain embodiments, the formulation may include a surfactant which is a polysorbate. In certain embodiments, the formulation may contain the detergent polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th edi., 1996). In certain embodiments, the formulation may contain between about 0.1 mg/mL and about 10 mg/mL of polysorbate 80, or between about 0.5 mg/mL and about 5 mg/mL. In certain embodiments, about 0.1% polysorbate 80 may be added in the formulation.

In embodiments, the protein product of the present disclosure is formulated as a liquid formulation. The liquid formulation may be presented at a 10 mg/mL concentration in either a USP/Ph Eur type I 50R vial closed with a rubber stopper and sealed with an aluminum crimp seal closure. The stopper may be made of elastomer complying with USP and Ph Eur. In certain embodiments vials may be filled with 61.2 mL of the protein product solution in order to allow an extractable volume of 60 mL. In certain embodiments, the liquid formulation may be diluted with 0.9% saline solution.

In certain embodiments, the liquid formulation of the disclosure may be prepared as a 10 mg/mL concentration solution in combination with a sugar at stabilizing levels. In certain embodiments the liquid formulation may be prepared in an aqueous carrier. In certain embodiments, a stabilizer may be added in an amount no greater than that which may result in a viscosity undesirable or unsuitable for intravenous administration. In certain embodiments, the sugar may be disaccharides, e.g., sucrose. In certain embodiments, the liquid formulation may also include one or more of a buffering agent, a surfactant, and a preservative.

In certain embodiments, the pH of the liquid formulation may be set by addition of a pharmaceutically acceptable acid and/or base. In certain embodiments, the pharmaceutically acceptable acid may be hydrochloric acid. In certain embodiments, the base may be sodium hydroxide.

In addition to aggregation, deamidation is a common product variant of peptides and proteins that may occur during fermentation, harvest/cell clarification, purification, drug substance/drug product storage and during sample analysis. Deamidation is the loss of NH3 from a protein forming a succinimide intermediate that can undergo hydrolysis. The succinimide intermediate results in a 17 daltons mass decrease of the parent peptide. The subsequent hydrolysis results in an 18 daltons mass increase. Isolation of the succinimide intermediate is difficult due to instability under aqueous conditions. As such, deamidation is typically detectable as 1 dalton mass increase. Deamidation of an asparagine results in either aspartic or isoaspartic acid. The parameters affecting the rate of deamidation include pH, temperature, solvent dielectric constant, ionic strength, primary sequence, local polypeptide conformation and tertiary structure. The amino acid residues adjacent to Asn in the peptide chain affect deamidation rates. Gly and Ser following an Asn in protein sequences results in a higher susceptibility to deamidation.

In certain embodiments, the liquid formulation of the present disclosure may be preserved under conditions of pH and humidity to prevent deamination of the protein product.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g. phosphate-buffered saline), sterile saline solution, Ringer's solution or dextrose solution.

A preservative may be optionally added to the formulations herein to reduce bacterial action. The addition of a preservative may, for example, facilitate the production of a multi-use (multiple-dose) formulation.

Intravenous (IV) formulations may be the preferred administration route in particular instances, such as when a patient is in the hospital after transplantation receiving all drugs via the IV route. In certain embodiments, the liquid formulation is diluted with 0.9% Sodium Chloride solution before administration. In certain embodiments, the diluted drug product for injection is isotonic and suitable for administration by intravenous infusion.

In certain embodiments, a salt or buffer components may be added in an amount of 10 mM-200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

A preservative may be optionally added to the formulations herein to reduce bacterial action. The addition of a preservative may, for example, facilitate the production of a multi-use (multiple-dose) formulation.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g. phosphate-buffered saline), sterile saline solution, Ringer's solution or dextrose solution.

This present disclosure could exist in a lyophilized formulation including the proteins and a lyoprotectant. The lyoprotectant may be sugar, e.g., disaccharides. In certain embodiments, the lycoprotectant may be sucrose or maltose. The lyophilized formulation may also include one or more of a buffering agent, a surfactant, a bulking agent, and/or a preservative.

The amount of sucrose or maltose useful for stabilization of the lyophilized drug product may be in a weight ratio of at least 1:2 protein to sucrose or maltose. In certain embodiments, the protein to sucrose or maltose weight ratio may be of from 1:2 to 1:5.

In certain embodiments, the pH of the formulation, prior to lyophilization, may be set by addition of a pharmaceutically acceptable acid and/or base. In certain embodiments the pharmaceutically acceptable acid may be hydrochloric acid. In certain embodiments, the pharmaceutically acceptable base may be sodium hydroxide.

Before lyophilization, the pH of the solution containing the protein of the present disclosure may be adjusted between 6 to 8. In certain embodiments, the pH range for the lyophilized drug product may be from 7 to 8.

In certain embodiments, a salt or buffer components may be added in an amount of 10 mM-200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

In certain embodiments, a “bulking agent” may be added. A “bulking agent” is a compound which adds mass to a lyophilized mixture and contributes to the physical structure of the lyophilized cake (e.g., facilitates the production of an essentially uniform lyophilized cake which maintains an open pore structure). Illustrative bulking agents include mannitol, glycine, polyethylene glycol and sorbitol. The lyophilized formulations of the present invention may contain such bulking agents.

A preservative may be optionally added to the formulations herein to reduce bacterial action. The addition of a preservative may, for example, facilitate the production of a multi-use (multiple-dose) formulation.

In certain embodiments, the lyophilized drug product may be constituted with an aqueous carrier. The aqueous carrier of interest herein is one which is pharmaceutically acceptable (e.g., safe and non-toxic for administration to a human) and is useful for the preparation of a liquid formulation, after lyophilization. Illustrative diluents include sterile water for injection (SWFI), bacteriostatic water for injection (BWFI), a pH buffered solution (e.g. phosphate-buffered saline), sterile saline solution, Ringer's solution or dextrose solution.

In certain embodiments, the lyophilized drug product of the current disclosure is reconstituted with either Sterile Water for Injection, USP (SWFI) or 0.9% Sodium Chloride Injection, USP. During reconstitution, the lyophilized powder dissolves into a solution.

In certain embodiments, the lyophilized protein product of the instant disclosure is constituted to about 4.5 mL water for injection and diluted with 0.9% saline solution (sodium chloride solution).

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The specific dose can be a uniform dose for each patient, for example, 50-5000 mg of protein. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (Schmitz et al., Clinica Chimica Acta 308: 43-53, 2001; Steimer et al., Clinica Chimica Acta 308: 33-41, 2001).

In general, dosages based on body weight are from about 0.01 μg to about 100 mg per kg of body weight, such as about 0.01 μg to about 100 mg/kg of body weight, about 0.01 μg to about 50 mg/kg of body weight, about 0.01 μg to about 10 mg/kg of body weight, about 0.01 μg to about 1 mg/kg of body weight, about 0.01 μg to about 100 μg/kg of body weight, about 0.01 μg to about 50 μg/kg of body weight, about 0.01 μg to about 10 μg/kg of body weight, about 0.01 μg to about 1 μg/kg of body weight, about 0.01 μg to about 0.1 μg/kg of body weight, about 0.1 μg to about 100 mg/kg of body weight, about 0.1 μg to about 50 mg/kg of body weight, about 0.1 μg to about 10 mg/kg of body weight, about 0.1 μg to about 1 mg/kg of body weight, about 0.1 μg to about 100 μg/kg of body weight, about 0.1 μg to about 10 μg/kg of body weight, about 0.1 μg to about 1 μg/kg of body weight, about 1 μg to about 100 mg/kg of body weight, about 1 μg to about 50 mg/kg of body weight, about 1 μg to about 10 mg/kg of body weight, about 1 μg to about 1 mg/kg of body weight, about 1 μg to about 100 μg/kg of body weight, about 1 μg to about 50 μg/kg of body weight, about 1 μg to about 10 μg/kg of body weight, about 10 μg to about 100 mg/kg of body weight, about 10 μg to about 50 mg/kg of body weight, about 10 μg to about 10 mg/kg of body weight, about 10 μg to about 1 mg/kg of body weight, about 10 μg to about 100 μg/kg of body weight, about 10 μg to about 50 μg/kg of body weight, about 50 μg to about 100 mg/kg of body weight, about 50 μg to about 50 mg/kg of body weight, about 50 μg to about 10 mg/kg of body weight, about 50 μg to about 1 mg/kg of body weight, about 50 μg to about 100 μg/kg of body weight, about 100 μg to about 100 mg/kg of body weight, about 100 μg to about 50 mg/kg of body weight, about 100 μg to about 10 mg/kg of body weight, about 100 μg to about 1 mg/kg of body weight, about 1 mg to about 100 mg/kg of body weight, about 1 mg to about 50 mg/kg of body weight, about 1 mg to about 10 mg/kg of body weight, about 10 mg to about 100 mg/kg of body weight, about 10 mg to about 50 mg/kg of body weight, about 50 mg to about 100 mg/kg of body weight.

Doses may be given once or more times daily, weekly, monthly or yearly, or even once every 2 to 20 years. Persons of ordinary skill in the art can easily estimate repetition rates for dosing based on measured residence times and concentrations of the targetable construct or complex in bodily fluids or tissues. Administration of the present invention could be intravenous, intraarterial, intraperitoneal, intramuscular, subcutaneous, intrapleural, intrathecal, intracavitary, by perfusion through a catheter or by direct intralesional injection. This may be administered once or more times daily, once or more times weekly, once or more times monthly, and once or more times annually.

The description above describes multiple aspects and embodiments of the invention. The patent application specifically contemplates all combinations and permutations of the aspects and embodiments.

### EXAMPLES

The invention now being generally described, will be more readily understood by reference to the following examples, which are included merely for purposes of illustration of certain aspects and embodiments of the present invention, and is not intended to limit the invention.

### Example 1—NKG2D Binding Domains Bind to NKG2D

### NKG2D Binding Domains Bind to Purified Recombinant NKG2D

The nucleic acid sequences of human, mouse or cynomolgus NKG2D ectodomains were fused with nucleic acid sequences encoding human IgG1 Fc domains and introduced into mammalian cells to be expressed. After purification, NKG2D-Fc fusion proteins were adsorbed to wells of microplates. After blocking the wells with bovine serum albumin to prevent non-specific binding, NKG2D binding domains were titrated and added to the wells pre-adsorbed with NKG2D-Fc fusion proteins. Primary antibody binding was detected using a secondary antibody which was conjugated to horseradish peroxidase and specifically recognizes a human kappa light chain to avoid Fc cross-reactivity. 3,3′,5,5′-Tetramethylbenzidine (TMB), a substrate for horseradish peroxidase, was added to the wells to visualize the binding signal, whose absorbance was measured at 450 nM and corrected at 540 nM. An NKG2D binding domain clone, an isotype control or a positive control (selected from SEQ ID NO: 37-40, or anti-mouse NKG2D clones MI-6 and CX-5 available at eBioscience) was added to each well. Light chain variable domain defined by SEQ ID NO:37 can be paired with heavy chain variable domain defined by SEQ ID NO:38 to form an antigen-binding site that can bind to NKG2D, as illustrated in U.S. Pat. No. 9,273,136. Alternatively, light chain variable domain defined by SEQ ID NO:39 can be paired with heavy chain variable domain defined by SEQ ID NO:40 to form an antigen-binding site that can bind to NKG2D, as illustrated in U.S. Pat. No. 7,879,985.

SEQ ID NO: 37
QSALTQPASVSGSPGQSITISCSGSSSNIGNNAVNWYQQLPGKAPKLLIY
YDDLLPSGVSDRFSGSKSGTSAFLAISGLQSEDEADYYCAAWDDSLNGPV
FGGGTKLTVL
SEQ ID NO: 38
QVQLVESGGGLVKPGGSLRLSCAASGFTFSSYGMHWVRQAPGKGLEWVAF
IRYDGSNKYYADSVKGRFTISRDNSKNTLYLQMNSLRAEDTAVYYCAKDR
GLGDGTYPDYWGQGTTVTVSS
SEQ ID NO: 39
EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPWTFG
QGTKVEIK
SEQ ID NO: 40
QVHLQESGPGLVKPSETLSLTCTVSDDSISSYYWSWIRQPPGKGLEWIGH
ISYSGSANYNPSLKSRVTISVDTSKNQFSLKLSSVTAADTAVYYCANWDD
AFNIWGQGTMVTVSS

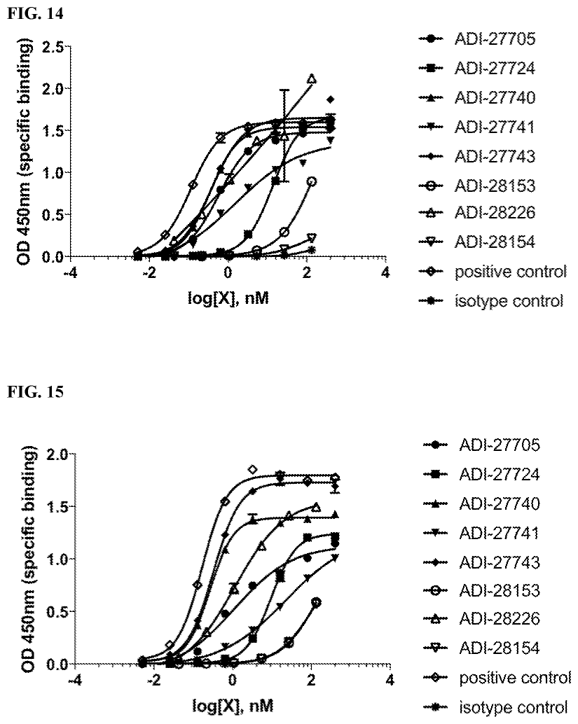

The isotype control showed minimal binding to recombinant NKG2D-Fc proteins, while the positive control bound strongest to the recombinant antigens. NKG2D binding domains produced by all clones demonstrated binding across human, mouse, and cynomolgus recombinant NKG2D-Fc proteins, although with varying affinities from clone to clone. Generally, each anti-NKG2D clone bound to human (FIG. 14) and cynomolgus recombinant NKG2D-Fc (FIG. 15) with similar affinity, but with lower affinity to mouse recombinant NKG2D-Fc.

### NKG2D Binding Domains Bind to Cells Expressing NKG2D

EL4 mouse lymphoma cell lines were engineered to express human or mouse NKG2D-CD3 zeta signaling domain chimeric antigen receptors. An NKG2D binding clone, an isotype control or a positive control was used at a 100 nM concentration to stain extracellular NKG2D expressed on the EL4 cells. The antibody binding was detected using fluorophore conjugated anti-human IgG secondary antibodies. Cells were analyzed by flow cytometry, and fold-over-background (FOB) was calculated using the mean fluorescence intensity (MFI) of NKG2D-expressing cells compared to parental EL4 cells.

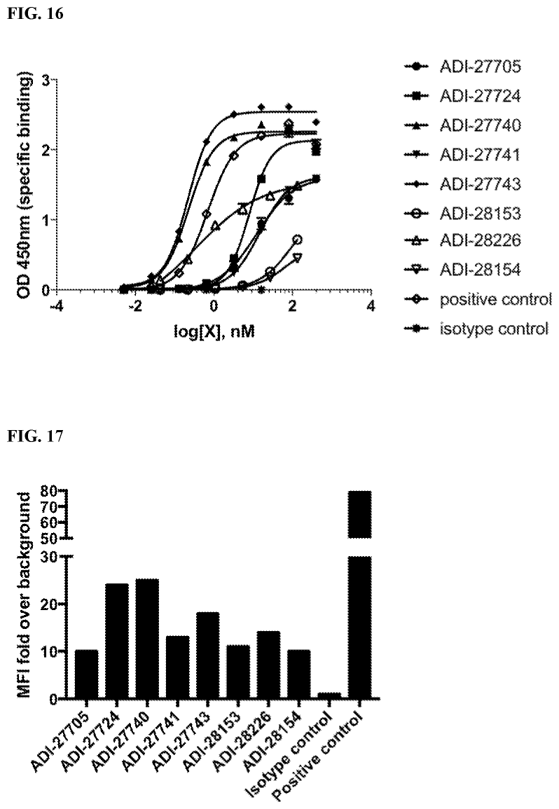

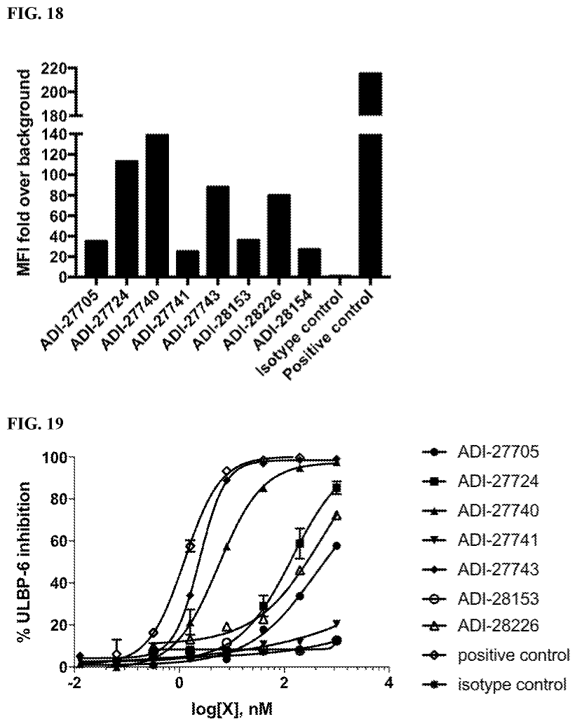

NKG2D binding domains produced by all clones bound to EL4 cells expressing human and mouse NKG2D. Positive control antibodies (selected from SEQ ID NO: 37-40, or anti-mouse NKG2D clones MI-6 and CX-5 available at eBioscience) gave the best FOB binding signal. The NKG2D binding affinity for each clone was similar between cells expressing human (FIG. 17) and mouse NKG2D (FIG. 16).

### Example 2—NKG2D Binding Domains Block Natural Ligand Binding to NKG2D

Competition with ULBP-6

Recombinant human NKG2D-Fc proteins were adsorbed to wells of a microplate, and the wells were blocked with bovine serum albumin reduce non-specific binding. A saturating concentration of ULBP-6-His-biotin was added to the wells, followed by addition of the NKG2D binding domain clones. After a 2-hour incubation, wells were washed and ULBP-6-His-biotin that remained bound to the NKG2D-Fc coated wells was detected by streptavidin conjugated to horseradish peroxidase and TMB substrate. Absorbance was measured at 450 nM and corrected at 540 nM. After subtracting background, specific binding of NKG2D binding domains to the NKG2D-Fc proteins was calculated from the percentage of ULBP-6-His-biotin that was blocked from binding to the NKG2D-Fc proteins in wells. The positive control antibody (selected from SEQ ID NO: 37-40) and various NKG2D binding domains blocked ULBP-6 binding to NKG2D, while isotype control showed little competition with ULBP-6 (FIG. 19).

Competition with MICA

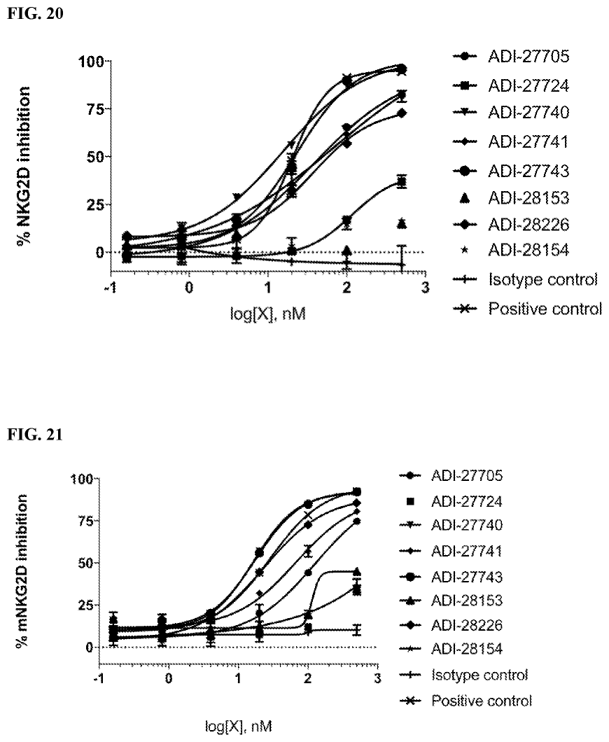

Recombinant human MICA-Fc proteins were adsorbed to wells of a microplate, and the wells were blocked with bovine serum albumin to reduce non-specific binding. NKG2D-Fc-biotin was added to wells followed by NKG2D binding domains. After incubation and washing, NKG2D-Fc-biotin that remained bound to MICA-Fc coated wells was detected using streptavidin-HRP and TMB substrate. Absorbance was measured at 450 nM and corrected at 540 nM. After subtracting background, specific binding of NKG2D binding domains to the NKG2D-Fc proteins was calculated from the percentage of NKG2D-Fc-biotin that was blocked from binding to the MICA-Fc coated wells. The positive control antibody (selected from SEQ ID NO: 37-40) and various NKG2D binding domains blocked MICA binding to NKG2D, while isotype control showed little competition with MICA (FIG. 20).

Competition with Rae-1 Delta

Recombinant mouse Rae-1delta-Fc (purchased from R&D Systems) was adsorbed to wells of a microplate, and the wells were blocked with bovine serum albumin to reduce non-specific binding. Mouse NKG2D-Fc-biotin was added to the wells followed by NKG2D binding domains. After incubation and washing, NKG2D-Fc-biotin that remained bound to Rae-1delta-Fc coated wells was detected using streptavidin-HRP and TMB substrate. Absorbance was measured at 450 nM and corrected at 540 nM. After subtracting background, specific binding of NKG2D binding domains to the NKG2D-Fc proteins was calculated from the percentage of NKG2D-Fc-biotin that was blocked from binding to the Rae-1delta-Fc coated wells. The positive control (selected from SEQ ID NOs:37-40, or anti-mouse NKG2D clones MI-6 and CX-5 available at eBioscience) and various NKG2D binding domain clones blocked Rae-1delta binding to mouse NKG2D, while the isotype control antibody showed little competition with Rae-1delta (FIG. 21).

### Example 3—NKG2D Binding Domain Clones Activate NKG2D

Nucleic acid sequences of human and mouse NKG2D were fused to nucleic acid sequences encoding a CD3 zeta signaling domain to obtain chimeric antigen receptor (CAR) constructs. The NKG2D-CAR constructs were then cloned into a retrovirus vector using Gibson assembly and transfected into expi293 cells for retrovirus production. EL4 cells were infected with viruses containing NKG2D-CAR together with 8 μg/mL polybrene. 24 hours after infection, the expression levels of NKG2D-CAR in the EL4 cells were analyzed by flow cytometry, and clones which express high levels of the NKG2D-CAR on the cell surface were selected.

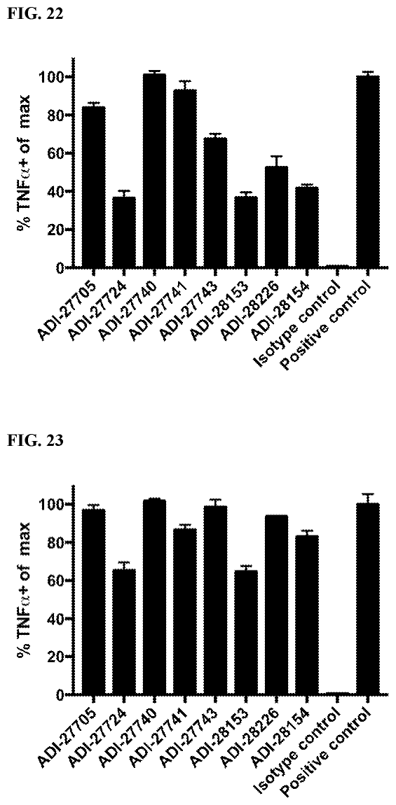

To determine whether NKG2D binding domains activate NKG2D, they were adsorbed to wells of a microplate, and NKG2D-CAR EL4 cells were cultured on the antibody fragment-coated wells for 4 hours in the presence of brefeldin-A and monensin. Intracellular TNF-alpha production, an indicator for NKG2D activation, was assayed by flow cytometry. The percentage of TNF-alpha positive cells was normalized to the cells treated with the positive control. All NKG2D binding domains activated both human (FIG. 22) and mouse (FIG. 23) NKG2D.

### Example 4—NKG2D Binding Domains Activate NK Cells

### Primary Human NK Cells

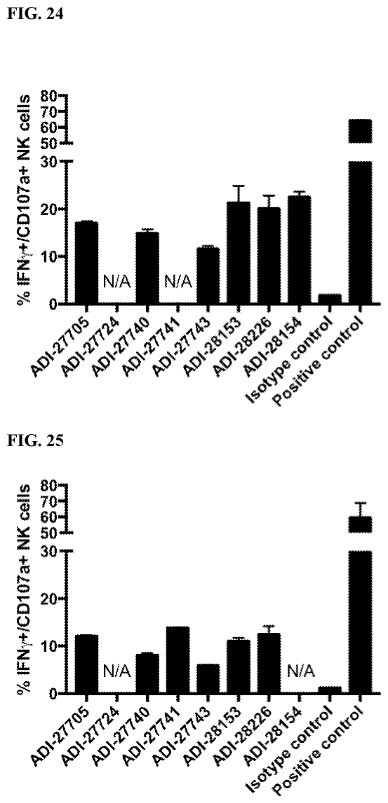

Peripheral blood mononuclear cells (PBMCs) were isolated from human peripheral blood buffy coats using density gradient centrifugation. NK cells (CD3⁻CD56⁺) were isolated using negative selection with magnetic beads from PBMCs, and the purity of the isolated NK cells was typically >95%. Isolated NK cells were then cultured in media containing 100 ng/mL IL-2 for 24-48 hours before they were transferred to the wells of a microplate to which the NKG2D binding domains were adsorbed, and cultured in the media containing fluorophore-conjugated anti-CD107a antibody, brefeldin-A, and monensin. Following culture, NK cells were assayed by flow cytometry using fluorophore-conjugated antibodies against CD3, CD56 and IFN-gamma. CD107a and IFN-gamma staining were analyzed in CD3⁻CD56⁺ cells to assess NK cell activation. The increase in CD107a/IFN-gamma double-positive cells is indicative of better NK cell activation through engagement of two activating receptors rather than one receptor. NKG2D binding domains and the positive control (selected from SEQ ID NOs:37-40) showed a higher percentage of NK cells becoming CD107a⁺ and IFN-gamma⁺ than the isotype control (FIGS. 24-25 represent two independent experiments each using a different donor's PBMC for NK cell preparation).

### Primary Mouse NK Cells

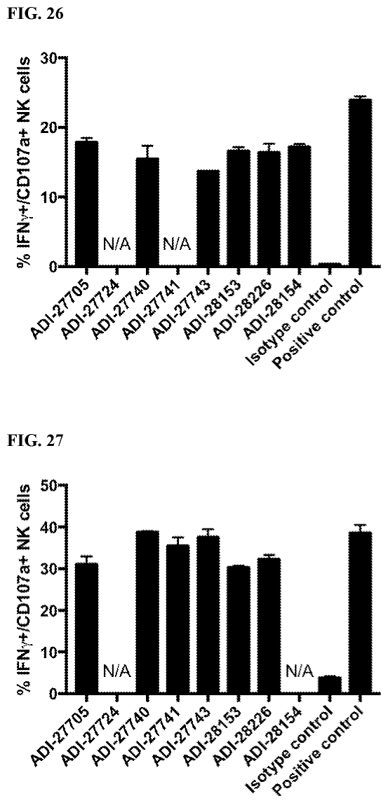

Spleens were obtained from C57Bl/6 mice and crushed through a 70 μm cell strainer to obtain single cell suspension. Cells were pelleted and resuspended in ACK lysis buffer (purchased from Thermo Fisher Scientific # A1049201; 155 mM ammonium chloride, 10 mM potassium bicarbonate, 0.01 mM EDTA) to remove red blood cells. The remaining cells were cultured with 100 ng/mL hIL-2 for 72 hours before being harvested and prepared for NK cell isolation. NK cells (CD3⁻NK1.1⁺) were then isolated from spleen cells using a negative depletion technique with magnetic beads with typically >90% purity. Purified NK cells were cultured in media containing 100 ng/mL mIL-15 for 48 hours before they were transferred to the wells of a microplate to which the NKG2D binding domains were adsorbed, and cultured in the media containing fluorophore-conjugated anti-CD107a antibody, brefeldin-A, and monensin. Following culture in NKG2D binding domain-coated wells, NK cells were assayed by flow cytometry using fluorophore-conjugated antibodies against CD3, NK1.1 and IFN-gamma. CD107a and IFN-gamma staining were analyzed in CD3⁻NK1.1⁺ cells to assess NK cell activation. The increase in CD107a/IFN-gamma double-positive cells is indicative of better NK cell activation through engagement of two activating receptors rather than one receptor. NKG2D binding domains and the positive control (selected from anti-mouse NKG2D clones MI-6 and CX-5 available at eBioscience) showed a higher percentage of NK cells becoming CD107a⁺ and IFN-gamma⁺ than the isotype control (FIGS. 26-27 represent two independent experiments each using a different mouse for NK cell preparation).

### Example 5—NKG2D Binding Domains Enable Cytotoxicity of Target Tumor Cells

Human and mouse primary NK cell activation assays demonstrate increased cytotoxicity markers on NK cells after incubation with NKG2D binding domains. To address whether this translates into increased tumor cell lysis, a cell-based assay was utilized where each NKG2D binding domain was developed into a monospecific antibody. The Fc region was used as one targeting arm, while the Fab region (NKG2D binding domain) acted as another targeting arm to activate NK cells. THP-1 cells, which are of human origin and express high levels of Fc receptors, were used as a tumor target and a Perkin Elmer DELFIA Cytotoxicity Kit was used. THP-1 cells were labeled with BATDA reagent, and resuspended at 10⁵/mL in culture media. Labeled THP-1 cells were then combined with NKG2D antibodies and isolated mouse NK cells in wells of a microtiter plate at 37° C. for 3 hours. After incubation, 20 μl of the culture supernatant was removed, mixed with 200 μl of Europium solution and incubated with shaking for 15 minutes in the dark. Fluorescence was measured over time by a PheraStar plate reader equipped with a time-resolved fluorescence module (Excitation 337 nm, Emission 620 nm) and specific lysis was calculated according to the kit instructions.

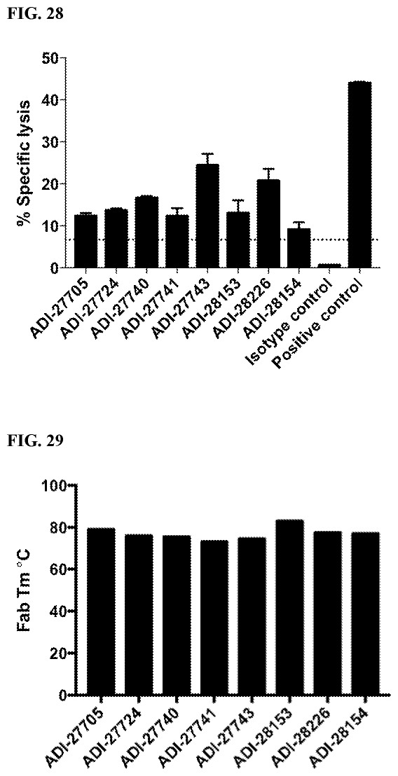

The positive control, ULBP-6—a natural ligand for NKG2D, showed increased specific lysis of THP-1 target cells by mouse NK cells. NKG2D antibodies also increased specific lysis of THP-1 target cells, while isotype control antibody showed reduced specific lysis. The dotted line indicates specific lysis of THP-1 cells by mouse NK cells without antibody added (FIG. 28).

### Example 6—NKG2D Antibodies Show High Thermostability

Melting temperatures of NKG2D binding domains were assayed using differential scanning fluorimetry. The extrapolated apparent melting temperatures are high relative to typical IgG1 antibodies (FIG. 29).

### Example 7—Multi-Specific Binding Proteins Display Enhanced Ability to Activate NK Cells

Peripheral blood mononuclear cells (PBMCs) were isolated from human peripheral blood buffy coats using density gradient centrifugation. NK cells (CD3⁻CD56⁺) were isolated using negative selection with magnetic beads from PBMCs, and the purity of the isolated NK cells was typically >95%. Isolated NK cells were then cultured in media containing 100 ng/mL IL-2 for 24-48 hours before they were transferred to the wells of a microplate to which multi-specific and bispecific binding proteins were adsorbed respectively, and cultured in the media containing fluorophore-conjugated anti-CD107a antibody, brefeldin-A, and monensin. Following culture, NK cells were assayed by flow cytometry using fluorophore-conjugated antibodies against CD3, CD56 and IFN-gamma. CD107a and IFN-gamma staining were analyzed in CD3⁻CD56⁺ cells to assess NK cell activation. The increase in CD107a/IFN-gamma double-positive cells is indicative of better NK cell activation. AL2.2 is a multi-specific binding protein containing HER2-binding domain (trastuzumab), NKG2D-binding domain (ULBP-6) and a human IgG1 Fc domain. It was made through a controlled Fab-arm exchange reaction (cFAE) starting from trastuzumab homodimer and ULBP-6-Fc homodimer (see Labrijn et al. Nature Protocols 9, 2450-2463). SC2.2 is single chain protein including an scFv derived from trastuzumab, and ULBP-6 (SEQ ID NO:41).

(SEQ ID NO: 41)
MAAAAIPALLLCLPLLFLLFGWSRARRDDPHSLCYDITVIPKFRPGPRWC
AVQGQVDEKTFLHYDCGNKTVTPVSPLGKKLNVTMAWKAQNPVLREVVDI
LTEQLLDIQLENYTPKEPLTLQARMSCEQKAEGHSSGSWQFSIDGQTFLL
FDSEKRMWTTVHPGARKMKEKWENDKDVAMSFHYISMGDCIGWLEDFLMG
MDSTLEPSAGAPLAMSSGTTQLRATATTLILCCLLIILPCFILPGI

### Example 8—Cytotoxic Activity of Rested Human NK Cells Mediated by TriNKETs, Monoclonal Antibodies, or Bispecific Antibodies Against HER2-Positive Cells

PBMCs were isolated from human peripheral blood buffy coats using density gradient centrifugation. Isolated PBMCs were washed and prepared for NK cell isolation. NK cells were isolated using a negative selection technique with magnetic beads; the purity of the isolated NK cells was typically >90% CD3-CD56+. Isolated NK cells were cultured in media containing 100 ng/mL IL-2 or were rested overnight without cytokine. IL-2-activated or rested NK cells were used the following day in cytotoxicity assays.

### DELFIA Cytotoxicity Assay:

Human cancer cell lines expressing a target of interest were harvested from culture, cells were washed with HBS, and were resuspended in growth media at 10⁶/mL for labeling with BATDA reagent (Perkin Elmer AD0116). Manufacturer instructions were followed for labeling of the target cells. After labeling, cells were washed 3× with HBS, and were resuspended at 0.5-1.0×10⁵/mL in culture media. To prepare the background wells an aliquot of the labeled cells was put aside, and the cells were spun out of the media. 100 μl of the media was carefully added to wells in triplicate to avoid disturbing the pelleted cells. 100 μl of BATDA labeled cells were added to each well of the 96-well plate. Wells were saved for spontaneous release from target cells, and wells were prepared for maximal lysis of target cells by addition of 1% Triton-X. Monoclonal antibodies or TriNKETs against the tumor target of interest were diluted in culture media and 50 μl of diluted mAb or TriNKET was added to each well. Rested and/or activated NK cells were harvested from culture, the cells were washed and were resuspended at 10⁵-2.0×10⁶/mL in culture media depending on the desired E:T ratio. 50 μl of NK cells were added to each well of the plate to make a total 200 μl culture volume. The plate was incubated at 37° C. with 5% CO2 for 2-3 hours before developing the assay.

After culturing for 2-3 hours, the plate was removed from the incubator and the cells were pelleted by centrifugation at 200 g for 5 minutes. 20 μl of culture supernatant was transferred to a clean microplate provided from the manufacturer and 200 μl of room temperature europium solution was added to each well. The plate was protected from the light and incubated on a plate shaker at 250 rpm for 15 minutes. The plate was read using either Victor 3 or SpectraMax i3X instruments. % Specific lysis was calculated as follows: % Specific lysis=((Experimental release−Spontaneous release)/(Maximum release−Spontaneous release))*100%.

Combination of Monoclonal Antibody and Bispecifc NK Cell Engager does not Recapitulate TriNKET Activity

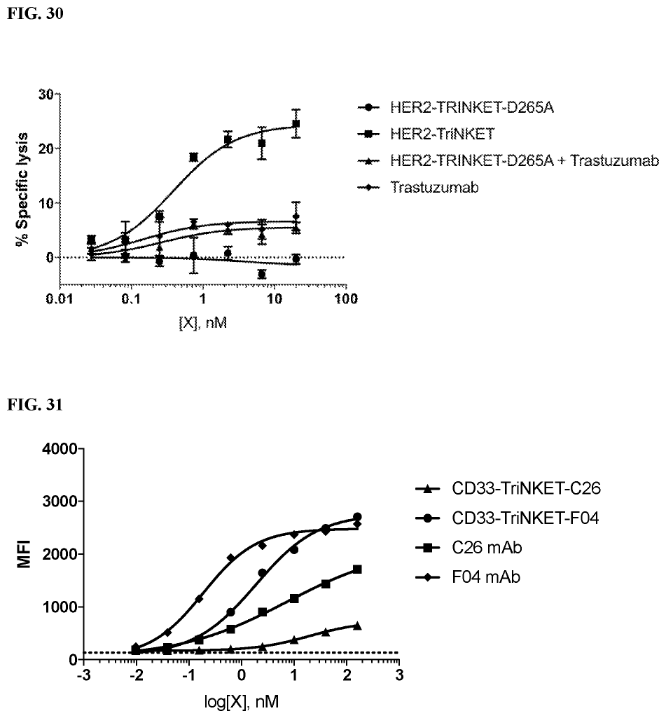

FIG. 30 shows the cytotoxic activity of rested human NK cells mediated by TriNKETs, monoclonal antibodies, or bispecific antibodies against the HER2-positive Colo-201 cell line. A TriNKET (ADI-29404 (F04)) targeting HER2 induced maximum lysis of Colo-201 cells by rested human NK cells. The D265A mutation was introduced into the CH2 domain of the TriNKET to abrogate FcR binding. The HER2-TriNKET (ADI-29404 (F04))-D265A fails to mediate lysis of Colo-201 cells, demonstrating the importance of dual targeting of CD16 and NKG2D on NK cells. To further demonstrate the importance of dual targeting on NK cells the monoclonal antibody Trastuzumab was used to target HER2 and mediate ADCC by NK cells, Trastuzumab alone was able to increase NK cell lysis of Colo-201 cells, but maximum lysis achieved by Trastuzumab alone was about 4× lower compared to the TriNKET. To understand the importance of having CD16 and NKG2D targeting on the same molecule, TriNKET (ADI-29404 (F04)) activity was compared to the activity of a bispecific antibody targeting HER2 and NKG2D combined with Trastuzumab. When used at equimolar concentrations the combination of bispecific and Trastuzumab was not able to mediate maximal lysis of Colo-201 cells by rested human NK cells. The failure of Trastuzumab+bispecific combination demonstrates the importance of containing the trispecific-binding of TriNKETs in one molecule

### Example 9—Multi-Specific Binding Proteins Bind to NKG2D

EL4 mouse lymphoma cell lines were engineered to express human NKG2D. Trispecific binding proteins (TriNKETs) that each contain an NKG2D-binding domain, a tumor-associated antigen binding domain (such as a CD33-, a HER2-, or a CD20-binding domain), and an Fc domain that binds to CD16 as shown in FIG. 1, were tested for their affinity to extracellular NKG2D expressed on EL4 cells. The binding of the multi-specific binding proteins to NKG2D was detected using fluorophore-conjugated anti-human IgG secondary antibodies. Cells were analyzed by flow cytometry, and fold-over-background (FOB) was calculated using the mean fluorescence intensity (MFI) of NKG2D-expressing cells compared to parental EL4 cells.

TriNKETs tested include CD33-TriNKET-C26 (ADI-28226 and a CD33-binding domain), CD33-TriNKET-F04 (ADI-29404 and a CD33-binding domain), HER2-TriNKET-C26 (ADI-28226 and a HER2-binding domain), HER2-TriNKET-F04 (ADI-29404 and a HER2-binding domain), CD20-TriNKET-C26 (ADI-28226 and a CD20-binding domain), and CD20-TriNKET-F04 (ADI-29404 and a CD20-binding domain). The HER2-binding domain used in the tested molecules was composed of a heavy chain variable domain and a light chain variable domain of Trastuzumab. The CD33-binding domain was composed of a heavy chain variable domain and a light chain variable domain listed below.

```
SEQ ID NO: 42:
QVQLVQSGAEVKKPGASVKVSCKASGYTFTDYVVHWVRQAPGQGLEWMGYINPY
ND
                                                              CDR1
GTKYNEKFKGRVTMTRDTSISTAYMELSRLRSDDTAVYYCARDYRYEVYGMDYWG
Q
    CDR2 CDR3
GTLVTVSS
SEQ ID NO: 43:
DIVLTQSPASLAVSPGQRATITCTASSSVNYIHWYQQKPGQPPKLLIYDTSKVASGVP
AR
                                                  CDR1 CDR1
FSGSGSGTDFTLTINPVEANDTANYYCQQWRSYPLTFGQGTKLEIK
                                                          CDR3
```

The CD20-binding domain used in the tested molecules was composed of a heavy chain variable domain and a light chain variable domain.

```
Rituximab_vH
(SEQ ID NO: 47)
QVQLQQPGAELVKPGASVKMSCKASGYTFTSYNMHWVKQTPGRGLEWIGAIYPGN
                                                              CDR1 CDR2
GDTSYNQKFKGKATLTADKSSSTAYMQLSSLTSEDSAVYYCARSTYYGGDWYFNV
                                                                                          CDR3
WGAGTTVTVSA
Rituximab_vL
(SEQ ID NO: 48)
QIVLSQSPAILSASPGEKVTMTCRASSSVSYIHWFQQKPGSSPKPWIYATSNLASGVP
                                                  CDR1 CDR2
VRFSGSGSGTSYSLTISRVEAEDAATYYCQQWTSNPPTFGGGTKLEIK
                                                                CDR3
```

### Example 10—Multi-Specific Binding Proteins Bind to Human Tumor Antigens

### Trispecific Binding Proteins Bind to CD33, HER2, and CD20

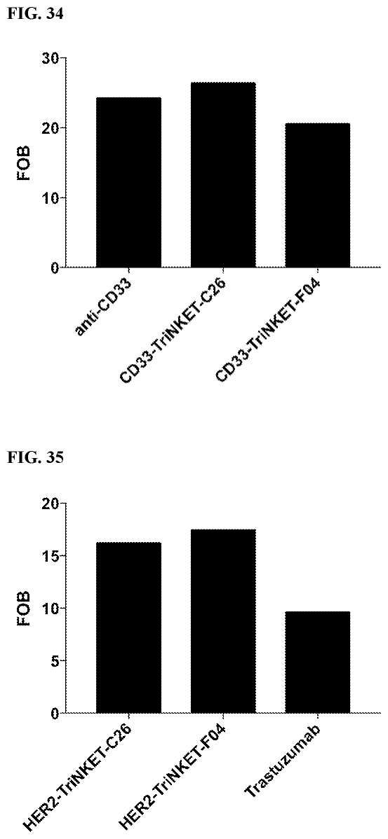

Human AML cell line MV4-11, expressing CD33 was used to assay the binding of TriNKETs to the tumor associated antigen. TriNKETs and the parental anti-CD33 monoclonal antibody were incubated with the cells, and the binding was detected using fluorophore-conjugated anti-human IgG secondary antibodies. Cells were analyzed by flow cytometry, and fold-over-background (FOB) was calculated using the mean fluorescence intensity (MFI) from TriNKETs and the parental monoclonal anti-CD33 antibody normalized to secondary antibody controls. CD33-TriNKET-C26, and CD33-TriNKET-F04 show comparable levels of binding to CD33 as compared with the parental anti-CD33 antibody (FIG. 34).

Human cancer cell lines expressing HER2 were used to assay the binding of TriNKETs to the tumor associated antigen. Renal cell carcinoma cell line 786-O expresses low levels of HER2. TriNKETs and optionally the parental anti-HER2 monoclonal antibody (Trastuzumab) were incubated with the cells, and the binding was detected using fluorophore-conjugated anti-human IgG secondary antibodies. Cells were analyzed by flow cytometry, and fold-over-background (FOB) was calculated using the mean fluorescence intensity (MFI) from TriNKETs and Trastuzumab normalized to secondary antibody controls. HER2-TriNKET-C26, and HER2-TriNKET-F04 show comparable levels of binding to HER2 expressed on 786-O cells as compared with Trastuzumab (FIG. 35).

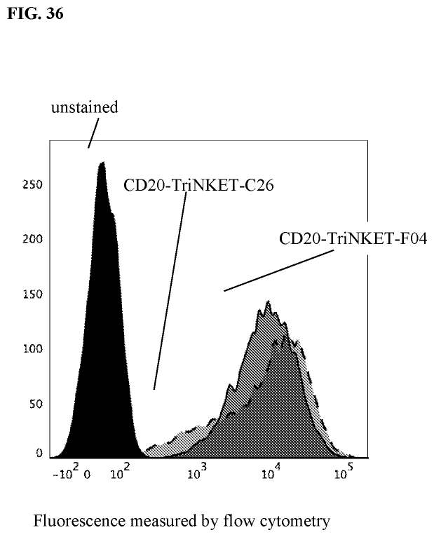

Raji human lymphoma cells expressing CD20 were used to assay the binding of TriNKETs to the tumor associated antigen CD20. TriNKETs were incubated with the cells, and the binding was detected using fluorophore-conjugated anti-human IgG secondary antibodies. Cells were analyzed by flow cytometry and histogram was plot. As shown in FIG. 36, CD20-TriNKET-C26 and CD20-TriNKET-F04 bind to CD20 equally well.

### Example 11—Multi-Specific Binding Proteins Activate NK Cells

Peripheral blood mononuclear cells (PBMCs) were isolated from human peripheral blood buffy coats using density gradient centrifugation. NK cells (CD3⁻CD56⁺) were isolated using negative selection with magnetic beads from PBMCs, and the purity of the isolated NK cells was typically >90%. Isolated NK cells were cultured in media containing 100 ng/mL IL-2 for activation or rested overnight without cytokine. IL-2-activated NK cells were used within 24-48 hours after activation.

Human cancer cells expressing a tumor antigen were harvested and resuspended in culture media at 2×10⁶/mL. Monoclonal antibodies or TriNKETs targeting the tumor antigen were diluted in culture media. Activated NK cells were harvested, washed, and resuspended at 2×10⁶/mL in culture media. Cancer cells were then mixed with monoclonal antibodies/TriNKETs and activated NK cells in the presence of IL-2. Brefeldin-A and monensin were also added to the mixed culture to block protein transport out of the cell for intracellular cytokine staining. Fluorophore-conjugated anti-CD107a was added to the mixed culture and the culture was incubated for 4 hours before samples were prepared for FACS analysis using fluorophore-conjugated antibodies against CD3, CD56 and IFN-gamma. CD107a and IFN-gamma staining was analyzed in CD3⁻CD56⁺ cells to assess NK cell activation. The increase in CD107a/IFN-gamma double-positive cells is indicative of better NK cell activation through engagement of two activating receptors rather than one receptor.

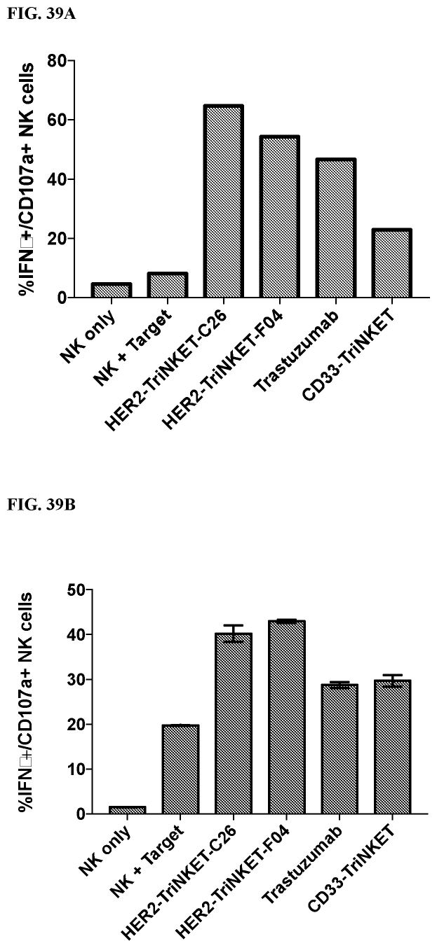

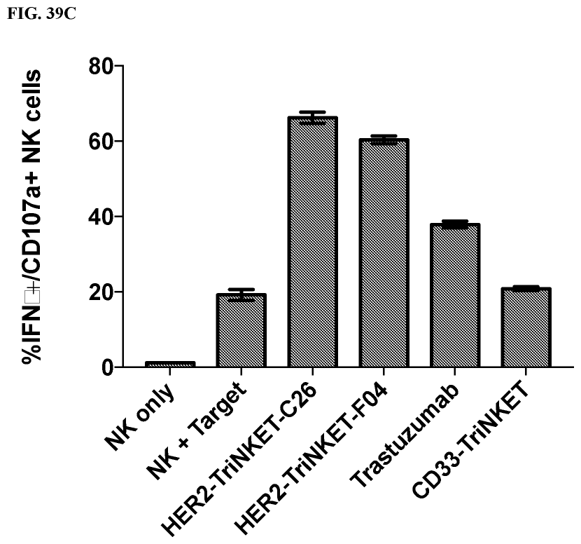

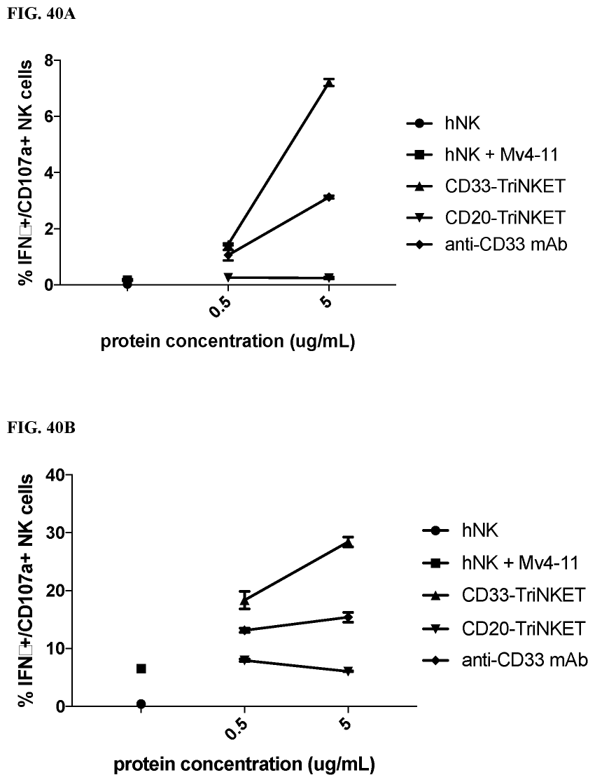

TriNKETs mediate activation of human NK cells co-cultured with HER2-expressing SkBr-3 cells (FIG. 39A), Colo201 cells (FIG. 39B), and HCC1954 cells (FIG. 39C) respectively as indicated by an increase of CD107a degranulation and IFN-gamma production. SkBr-3 cells and HCC1954 cells have high levels of surface HER2 expression, and Colo201 has medium HER2 expression. Compared to the monoclonal antibody trastuzumab, TriNKETs show superior activation of human NK cells in the presence of human cancer cells. NK cells alone, NK cells plus SkBr-3 cells are used as negative controls.

TriNKETs (C26-TriNKET-HER2 and F04-TriNKET-HER2) mediate activation of human NK cells co-cultured with CD33-expressing human AML Mv4-11 cells showed an increase of CD107a degranulation and IFN-gamma production. Compared to the monoclonal anti-CD33 antibody, TriNKETs (C26-TriNKET-HER2 and F04-TriNKET-HER2) showed superior activation of human NK cells in the presence of human cancer cells expressing HER2 (FIGS. 39A-39C).

Primary Human NK Cells are Activated by TriNKETs in Co-Culture with Target Expressing Human Cancer Cell Lines

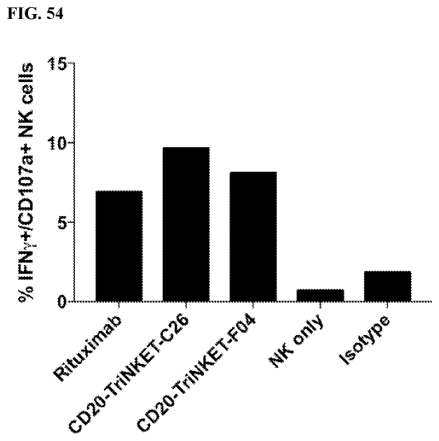

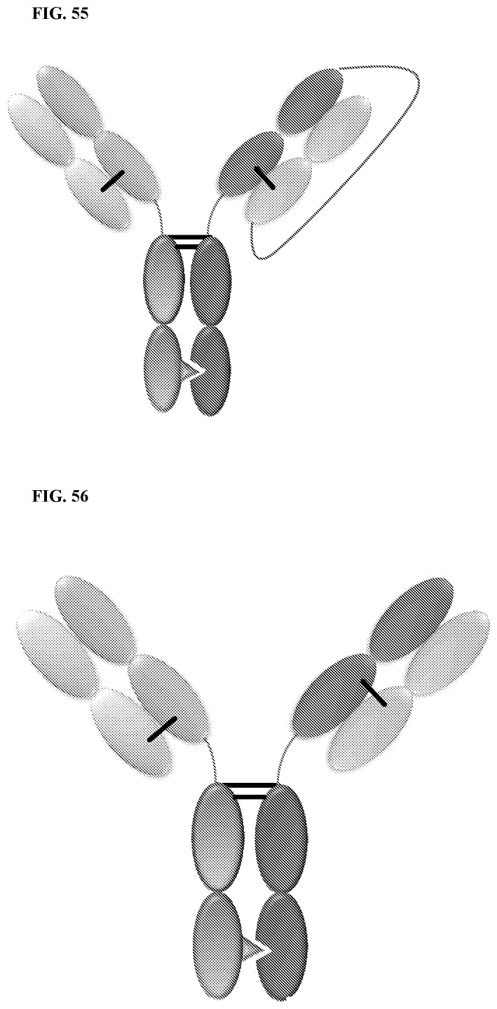

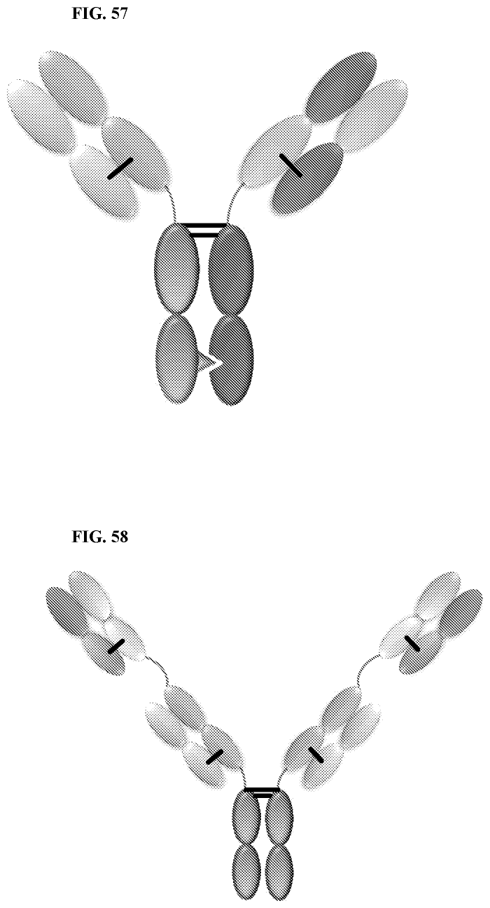

Co-culturing primary human NK cells with CD20-positive human cancer cells resulted in TriNKET-mediated activation of primary human NK cells (FIG. 54). TriNKETs targeting CD20 (e.g., C26-TriNKET-CD20 and F04-TriNKET-CD20), mediated activation of human NK cells co-cultured with CD20-positive Raji cells, as indicated by an increase in CD107a degranulation and IFNγ cytokine production (FIG. 54). Compared to the monoclonal antibody Rituximab, both TriNKETs (e.g., C26-TriNKET-CD20 and F04-TriNKET-CD20) showed superior activation of human NK cells (FIG. 54).

### Example 12—Trispecific Binding Proteins Enable Cytotoxicity of Target Cancer Cells

Peripheral blood mononuclear cells (PBMCs) were isolated from human peripheral blood buffy coats using density gradient centrifugation. NK cells (CD3⁻CD56⁺) were isolated using negative selection with magnetic beads from PBMCs, and the purity of the isolated NK cells was typically >90%. Isolated NK cells were cultured in media containing 100 ng/mL IL-2 for activation or rested overnight without cytokine. IL-2-activated or rested NK cells were used the following day in cytotoxicity assays.

In order to test the ability of human NK cells to lyse cancer cells in the presence of TriNKETs, a cyto Tox 96 non-radioactive cytotoxicity assay from Promega (G1780) was used according to the manufacturer's instructions. Briefly, human cancer cells expressing a tumor antigen were harvested, washed, and resuspended in culture media at 1-2×10⁵/mL. Rested and/or activated NK cells were harvested, washed, and resuspended at 10⁵-2.0×10⁶/mL in the same culture media as that of the cancer cells. In each well of a 96 well plate, 50 μl of the cancer cell suspension was mixed with 50 μl of NK cell suspension with or without TriNKETs targeting the tumor antigen expressed on the cancer cells. After incubation at 37° C. with 5% CO₂ for 3 hours and 15 minutes, 10× lysis buffer was added to wells containing only cancer cells, and to wells containing only media for the maximum lysis and negative reagent controls, respectively. The plate was then placed back into the incubator for an additional 45 minutes to reach a total of 4-hour incubation. Cells were then pelleted, and the culture supernatant was transferred to a new 96 well plate and mixed with a substrate for development. The new plate was incubated for 30 minutes at room temperature, and the absorbance was read at 492 nm on a SpectraMax i3x. Percentage of specific lysis of the cancer cells was calculated as follows: % Specific lysis=((experimental lysis−spontaneous lysis from NK cells alone−spontaneous lysis from cancer cells alone)/(Maximum lysis−negative reagent control))×100%.

TriNKETs mediate cytotoxicity of human NK cells against the CD33 positive Molm-13 human AML cell line. As shown in FIG. 45B, rested human NK cells were mixed with Molm-13 cancer cells, and TriNKETs (e.g., C26-TriNKET-CD33 and F04-TriNKET-CD33) are able to enhance the cytotoxic activity of rested human NK cells in a dose-responsive manner against the cancer cells. The dotted line indicates cytotoxic activity of rested NK cells without TriNKETs. Activated human NK cells were mixed with Molm-13 cancer cells, and TriNKETs enhance the cytotoxic activity of activated human NK cells even further, compared to an anti-CD33 antibody, in a dose-responsive manner against the cancer cells (FIG. 45B).

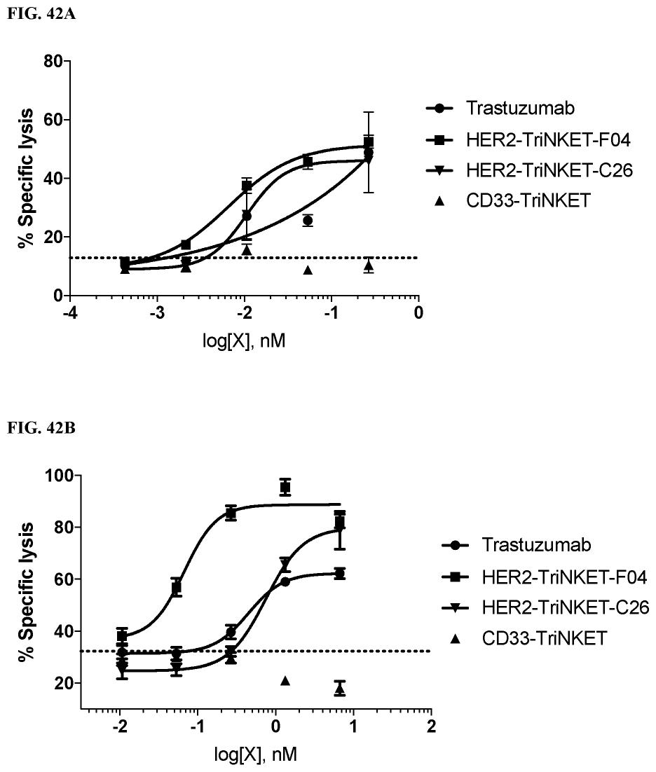

TriNKETs enhance NK cell cytotoxicity against targets with low surface expression compared to the cytotoxic activity of trastuzumab, an anti-HER2 monoclonal antibody. Rested human NK cells were mixed with high HER2-expressing SkBr tumor cells and low HER2-expressing 786-0 cancer cells, and TriNKETs' ability to enhance the cytotoxic activity of rested human NK cells against the high and low HER2-expressing cancer cells in a dose-responsive manner was assayed. Dotted lines in FIG. 42A and FIG. 42B indicate the cytotoxic activity of rested NK cells against the cancer cells in the absence of TriNKETs. As shown in FIG. 42B, upon mixing activated human NK cells with low HER2-expressing 786-0 cells, and TriNKET (e.g., CD26-TriNKET-HER2 and F04-TriNKET-HER2) dose-responsive cytotoxic activity of activated human NK cells against the cancer cells was observed.

### Example 13

Synergistic activation of human NK cells by cross-linking NKG2D and CD 16 was investigated.

### Primary Human NK Cell Activation Assay

Peripheral blood mononuclear cells (PBMCs) were isolated from peripheral human blood buffy coats using density gradient centrifugation. NK cells were purified from PBMCs using negative magnetic beads (StemCell #17955). NK cells were >90% CD3⁻CD56⁺ as determined by flow cytometry. Cells were then expanded 48 hours in media containing 100 ng/mL hIL-2 (Peprotech #200-02) before use in activation assays. Antibodies were coated onto a 96-well flat-bottom plate at a concentration of 2 μg/ml (anti-CD16, Biolegend #302013) and 5 μg/mL (anti-NKG2D, R&D # MAB139) in 100 μl sterile PBS overnight at 4° C. followed by washing the wells thoroughly to remove excess antibody. For the assessment of degranulation IL-2-activated NK cells were resuspended at 5×10⁵ cells/ml in culture media supplemented with 100 ng/mL hIL2 and 1 μg/mL APC-conjugated anti-CD107a mAb (Biolegend #328619). 1×10⁵ cells/well were then added onto antibody coated plates. The protein transport inhibitors Brefeldin A (BFA, Biolegend #420601) and Monensin (Biolegend #420701) were added at a final dilution of 1:1000 and 1:270 respectively. Plated cells were incubated for 4 hours at 37° C. in 5% CO₂. For intracellular staining of IFN-γ NK cells were labeled with anti-CD3 (Biolegend #300452) and anti-CD56 mAb (Biolegend #318328) and subsequently fixed and permeabilized and labeled with anti-IFN-γ mAb (Biolegend #506507). NK cells were analyzed for expression of CD107a and IFN-γ by flow cytometry after gating on live CD56⁺CD3⁻ cells.

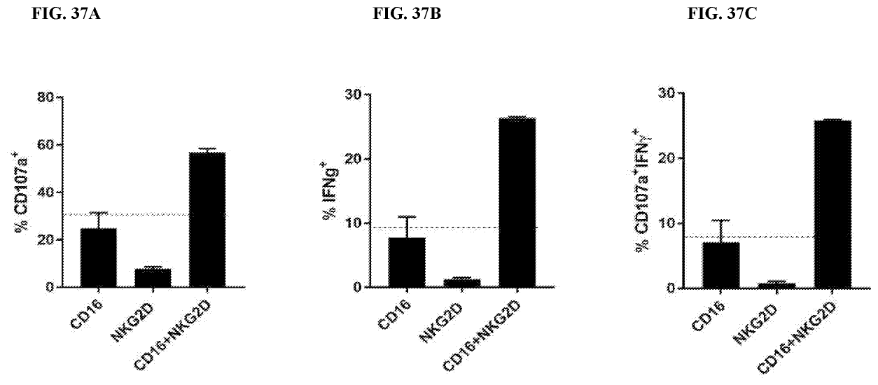

To investigate the relative potency of receptor combination, crosslinking of NKG2D or CD16 and co-crosslinking of both receptors by plate-bound stimulation was performed. As shown in FIG. 37 (FIGS. 37A-37C), combined stimulation of CD16 and NKG2D resulted in highly elevated levels of CD107a (degranulation) (FIG. 37A) and/or IFN-γ production (FIG. 37B). Dotted lines represent an additive effect of individual stimulations of each receptor.

CD107a levels and intracellular IFN-γ production of IL-2-activated NK cells were analyzed after 4 hours of plate-bound stimulation with anti-CD16, anti-NKG2D or a combination of both monoclonal antibodies. Graphs indicate the mean (n=2)±SD. FIG. 37A demonstrates levels of CD107a; FIG. 37B demonstrates levels of IFNγ; FIG. 37C demonstrates levels of CD107a and IFN-γ. Data shown in FIGS. 37A-37C are representative of five independent experiments using five different healthy donors.

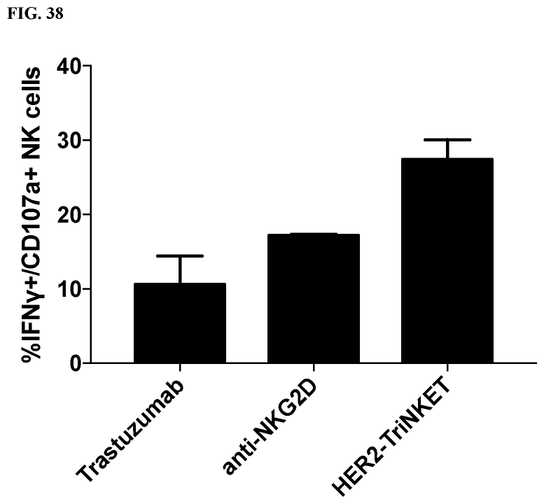

CD107a degranulation and intracellular IFN-γ production of IL-2-activated NK cells were analyzed after 4 hours of plate-bound stimulation with trastuzumab, anti-NKG2D, or a TriNKET derived from the binding domains of trastuzumab and the anti-NKG2D antibody (FIG. 38). In all cases antibodies tested were of the human IgG1 isotype. Graphs indicate the mean (n=2)±SD.

### Example 14

### Assessment of TriNKET Binding to Cell-Expressed Human NKG2D

EL4 cells transduced with human NKG2D were used to test binding to cell-expressed human NKG2D. TriNKETs were diluted to 20 μg/mL, and then diluted serially. The mAb or TriNKET dilutions were used to stain cells, and binding of the TriNKET or mAb was detected using a fluorophore-conjugated anti-human IgG secondary antibody. Cells were analyzed by flow cytometry, binding MFI was normalized to secondary antibody controls to obtain fold over background values.

### Assessment of TriNKET Binding to Cell-Expressed Human Cancer Antigens

Human cancer cell lines expressing either CD33 or HER2 were used to assess tumor antigen binding of TriNKETs derived from different NKG2D targeting clones. The human AML cell line MV4-11 was used to assess binding of TriNKETs to cell-expressed CD33. The human renal cell carcinoma cell line 786-0 expresses low levels of HER2 and was used to assess TriNKET binding to cell-expressed HER2. TriNKETs were diluted to 20 μg/mL, and were incubated with the respective cells. Binding of the TriNKET was detected using a fluorophore-conjugated anti-human IgG secondary antibody. Cells were analyzed by flow cytometry, binding MFI to cell expressed CD33 and HER2 was normalized to secondary antibody controls to obtain fold over background values.

### Determination of Antibody Binding Capacity of Human HER2-Positive Cancer Cell Lines

Antibody binding capacity (ABC) of HER2-positive human cancer cell lines was measured. The Quantum Simply Cellular kit from Bangs Lab was used (#815), and the manufacturer instructions were followed for the preparation of antibody labeled beads. Briefly, each of the four populations of beads were stained with a saturating amount of anti-HER2 antibody, and the cell populations were also stained with a saturating amount of the same antibody. Sample data was acquired for each bead population, as well as the cell populations. The QuickCal worksheet, provided with the kit, was used for the generation of a standard curve and extrapolation of ABC values for each of the cell lines.

### Activation of Primary NK Cells by TriNKETs

PBMCs were isolated from human peripheral blood buffy coats using density gradient centrifugation. Isolated PBMCs were washed and prepared for NK cell isolation. NK cells were isolated using a negative selection technique with magnetic beads; the purity of isolated NK cells was typically >90% CD3-CD56+. Isolated NK cells were cultured in media containing 100 ng/mL IL-2 for activation or rested overnight without cytokine. IL-2-activated NK cells were used 24-48 hours later; rested NK cells were always used the day after purification.

Human cancer cell lines expressing a cancer target of interest were harvested from culture, and cells were adjusted to 2×10⁶/mL. Monoclonal antibodies or TriNKETs targeting the cancer target of interest were diluted in culture media. Rested and/or activated NK cells were harvested from culture, cells were washed, and were resuspended at 2×10⁶/mL in culture media. IL-2, and fluorophore-conjugated anti-CD107a were added to the NK cells for the activation culture. Brefeldin-A and monensin were diluted into culture media to block protein transport out of the cell for intracellular cytokine staining. Into a 96-well plate 50 μl of tumor targets, mAbs/TriNKETs, BFA/monensin, and NK cells were added for a total culture volume of 200 The plate was cultured for 4 hours before samples were prepared for FACS analysis.

Following the 4 hour activation culture, cells were prepared for analysis by flow cytometry using fluorophore-conjugated antibodies against CD3, CD56 and IFNγ. CD107a and IFNγ staining was analyzed in CD3-CD56+ populations to assess NK cell activation.

### Primary Human NK Cell Cytotoxicity Assay

PBMCs were isolated from human peripheral blood buffy coats using density gradient centrifugation. Isolated PBMCs were washed and prepared for NK cell isolation. NK cells were isolated using a negative selection technique with magnetic beads, purity of isolated NK cells was typically >90% CD3-CD56+. Isolated NK cells were cultured in media containing 100 ng/mL IL-2 or were rested overnight without cytokine. IL-2-activated or rested NK cells were used the following day in cytotoxicity assays.

### Cyto Tox 96 LHD Release Assay:

The ability of human NK cells to lyse tumor cells was measured with or without the addition of TriNKETs using the cyto Tox 96 non-radioactive cytotoxicity assay from Promega (G1780). Human cancer cell lines expressing a cancer target of interest were harvested from culture, cells were washed with PBS, and were resuspended in growth media at 1-2×10⁵/mL for use as target cells. 50 μl of the target cell suspension were added to each well. Monoclonal antibodies or TriNKETs targeting a cancer antigen of interest were diluted in culture media, 50 μl of diluted mAb or TriNKET were added to each well. Rested and/or activated NK cells were harvested from culture, cells were washed, and were resuspended at 10⁵-2.0×10⁶/mL in culture media depending on the desired E:T ratio. 50 μl of NK cells were added to each well of the plate to make a total of 150 μl culture volume. The plate was incubated at 37° C. with 5% CO2 for 3 hours and 15 minutes. After the incubation, 10× lysis buffer was added to wells of target cells alone, and to wells containing media alone, for maximum lysis and volume controls. The plate was then placed back into the incubator for an additional 45 minutes, to make to total of 4 hours of incubation before development.

After incubation, the plate was removed from the incubator and the cells were pelleted by centrifugation at 200 g for 5 minutes. 50 μl of culture supernatant were transferred to a clean microplate and 50 μl of substrate solution were added to each well. The plate was protected from the light and incubated for 30 minutes at room temperature. 50 μl of stop solution were added to each well, and absorbance was read at 492 nm on a SpectraMax i3x. % Specific lysis was calculated as follows: % Specific lysis=((Experimental release−Spontaneous release from effector−Spontaneous release from target)/(Maximum release−Spontaneous release))*100%.

### DELFIA Cytotoxicity Assay:

Human cancer cell lines expressing a target of interest were harvested from culture, cells were washed with PBS, and were resuspended in growth media at 10⁶/mL for labeling with BATDA reagent (Perkin Elmer AD0116). Manufacturer instructions were followed for labeling of the target cells. After labeling cells were washed 3× with PBS, and were resuspended at 0.5-1.0×10⁵/mL in culture media. To prepare the background wells an aliquot of the labeled cells was put aside, and the cells were spun out of the media. 100 μl of the media were carefully added to wells in triplicate to avoid disturbing the pelleted cells. 100 μl of BATDA labeled cells were added to each well of the 96-well plate. Wells were saved for spontaneous release from target cells, and wells were prepared for max lysis of target cells by addition of 1% Triton-X. Monoclonal antibodies or TriNKETs against the tumor target of interest were diluted in culture media and 50 μl of diluted mAb or TriNKET were added to each well. Rested and/or activated NK cells were harvested from culture, cells were washed, and were resuspended at 10⁵-2.0×10⁶/mL in culture media depending on the desired E:T ratio. 50 μl of NK cells were added to each well of the plate to make a total of 200 μl culture volume. The plate was incubated at 37° C. with 5% CO2 for 2-3 hours before developing the assay.

After culturing for 2-3 hours, the plate was removed from the incubator and the cells were pelleted by centrifugation at 200 g for 5 minutes. 20 μl of culture supernatant was transferred to a clean microplate provided from the manufacturer, 200 μl of room temperature europium solution was added to each well. The plate was protected from the light and incubated on a plate shaker at 250 rpm for 15 minutes. Plate was read using either Victor 3 or SpectraMax i3X instruments. % Specific lysis was calculated as follows: % Specific lysis=((Experimental release−Spontaneous release)/(Maximum release−Spontaneous release))*100%.

### Long Term Human Pbmc Cytotoxicity Assay:

SkBr-3 target cells were labeled with BacMam 3.0 NucLight Green (#4622) to allow for tracking of the target cells. The manufacturer's protocol was followed for labeling of SkBr-3 target cells. Annexin V Red (Essen Bioscience #4641) was diluted and prepared according to the manufacturer's instructions. Monoclonal antibodies or TriNKETs were diluted into culture media. 50 μl of mAbs or TriNKETs, Annexin V, and rested NK cells were added to wells of a 96 well plate already containing labeled SkBr-3 cells; 50 ul of complete culture media was added for a total of 200 μl culture volume.

Image collection was setup on the IncuCyte S3. Images for the phase, green, and red channels were collected every hour, with 2 images per well. Image analysis was done using the IncuCyte S3 software. Masks for the green and red channels were created to count the number of tumor cells, and annexin V positive cells respectively. To calculate the % annexin V positive Mv4-11 target cells the following formula was used. % Annexin V positive SkBr-3 cells=((overlap object count)/(green object count))*100%.

Comparing a TriNKET that Targets HER+ Cancer Cells with SC2.2

A TriNKET targeting HER2 is more effective than Trastuzumab at reducing SkBr-3 cell number, and only 60% of the cells from time zero were left after 60 hours. A TriNKET of the present disclosure that targets HER2-expressing tumor/cancer cells is more effective than SC2.2—a single chain bispecific molecule built from an scFv derived from trastuzumab linked to ULBP-6, a ligand for NKG2D. SC2.2 binds HER2+ cancer cells and NKG2D+NK cells simultaneously. Therefore, effectiveness of SC2.2 in reducing HER2+ cancer cell number was investigated. In vitro activation and cytotoxity assays demonstrated that SC2.2 was effective in activating and killing NK cells. However, SC2.2 failed to demonstrate efficacy in the RMA/S-HER2 subcutaneous tumor model. The efficacy of SC2.2 was also tested in vivo using an RMA/S-HER2 overexpressing syngeneic mouse model. In this mouse model, SC2.2 failed to demonstrate control of tumor growth compared to vehicle control. Thus, although SC2.2 was able to activate and kill NK cells, and binds to HER2+ cancer cells, these properties were insufficient to effectively control HER2+ tumor growth.

### Assessment of SC2.2 Serum Half-Life in C57Bl/6 Mice

To determine the serum half-life of SC2.2 in C57Bl/6 mice, SC2.2 was labeled with a fluorescent tag to track its concentration in vivo. SC2.2 was labeled with IRDye 800CW (Licor #929-70020). The labeled protein was injected intravenously into 3 C57Bl/6 mice, blood was taken from each mouse at the indicated time points. After collection blood was centrifuged at 1000 g for 15 mins and serum was collected from each sample and stored at 4 C until all time points were collected.

Serum was imaged using an Odyssey CLx infrared imaging system, the fluorescent signal from the 800 channel was quantified using Image J software. Image intensities were normalized to the first time point, and the data was fit to a biphasic decay equation. In this experimental system the beta half-life of SC2.2 was calculated to be around 7 hours.

### In Vivo Testing of SC2.2 Against RMA/S-HER2 Subcutaneous Tumors

An in vivo study was designed according to FIG. 48 to test the efficacy of SC2.2 against subcutaneous RMA/S-HER2 tumors. 10⁶ RMA/S cells transduced with human HER2 were injected subcutaneously into the flank of 20 C57Bl/6 mice. Starting day 2 after tumor innoculation SC2.2 was dosed daily via IP injection. SC2.2 was dosed at a high and a low concentrations along with a vehicle control. Starting day 4 after tumor innoculation tumors were measured Monday, Wednesday, and Friday for the duration of the study. Tumor volume was calculated using the following formula: Tumor volume=Length×width×height

### TriNKETs Bind to Cells Expressing Human NKG2D

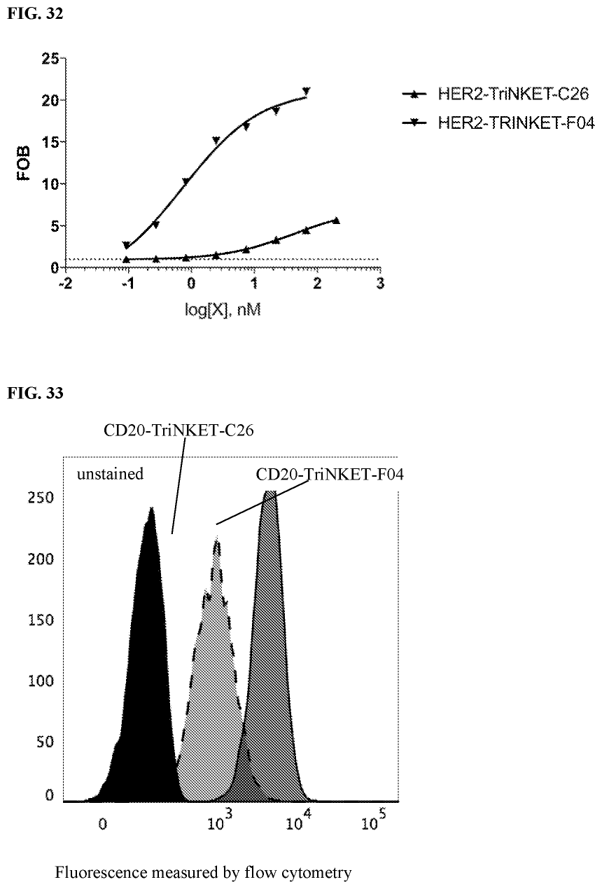

The ability of a TriNKET to bind cells expressing human NKG2D was determined. FIG. 31 and FIG. 32 show dose responsive binding of two TriNKETs containing different NKG2D-binding domains. FIG. 31 shows binding of the two TriNKETs when a CD33-binding domain is used as the second targeting arm. FIG. 32 shows the same two NKG2D-binding domains now paired with a HER2 second targeting arm. The six NKG2D-binding domains retain the same binding profile with both tumor targeting domains.

### TriNKETs Bind to Cells Expressing Human Cancer Antigens

The ability of a TriNKET to bind cells expressing human cancer antigens was determined. FIG. 34 and FIG. 35 show binding of TriNKETs to cell-expressed CD33 (FIG. 34) and HER2 (FIG. 35). TriNKET binding to cell-expressed antigen was consistent between NKG2D-binding domains. TriNKETs bound to comparable levels as the parental monoclonal antibody.

### Antibody Binding Capacity of Human HER2-Positive Cancer Cell Lines

Table 13 shows the results of HER2 surface quantification. SkBr-3 and HCC1954 cells were identified to have high (+++) levels of surface HER2. ZR-75-1 and Colo201 showed medium levels (++) of surface HER2, and 786-O showed the lowest level of HER2 (+).

TABLE 13
ABC of HER2-positive cancer cell lines

| Cell Line | HER2 expression | ABC |
|---|---|---|
| 786-0 | Low | 28,162 |
| Colo201 | Medium | 273,568 |
| ZR-75-1 | Medium | 281,026 |
| SkBr-3 | High | 6,820,532 |
| HCC1954 | High | 10,569,869 |

Primary Human NK Cells are Activated by TriNKETs in Co-Culture with Human Cancer Lines Expressing Varying Levels of HER2

FIGS. 39A-39C show that TriNKETs and trastuzumab were able to activate primary human NK cells in co-culture with HER2-positive human tumor cells, indicated by an increase in CD107a degranulation and IFNγ cytokine production. Compared to the monoclonal antibody trastuzumab, both TriNKETs showed superior activation of human NK cells with a variety of human HER2 cancer cells.

FIG. 39A shows that human NK cells are activated by TriNKETs when cultured with SkBr-3 cells. FIG. 39B shows that human NK cells are activated by TriNKETs when cultured with Colo201 cells. FIG. 39C shows that human NK cell are activated by TriNKETs when cultured with HCC1954 cells.

### TriNKETs Enhance Activity of Rested and IL-2-Activated Human NK Cells

FIGS. 40A-40B show TriNKET-mediated activation of rested or IL-2-activated human NK cells in co-culture with the CD33-expressing human AML cell line MV4-11. FIG. 40A shows TriNKET-mediated activation of resting human NK cells. FIG. 40B shows TriNKET-mediated activation of IL-2-activated human NK cells from the same donor. Rested NK cells showed less background IFNγ production and CD107a degranulation, than IL-2-activated NK cells. Rested NK cells showed a greater change in IFNγ production and CD107a degranulation compared to IL-2-activated NK cells. IL-2-activated NK cells showed a greater percentage of cells becoming IFNγ+; CD107a+ after stimulation with TriNKETs.

### TriNKETs Enhance Cytotoxicity of Rested and IL-2-Activated Human NK Cells

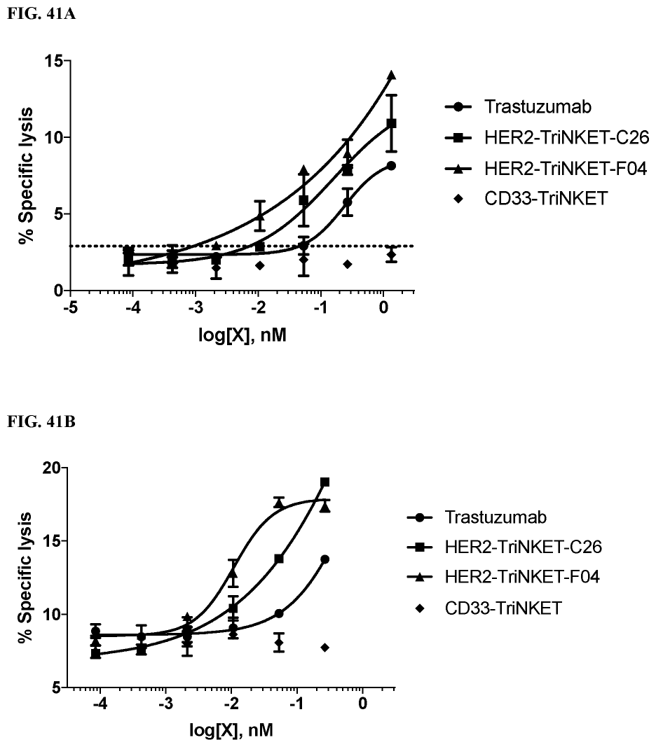

FIGS. 41A-41B show TriNKET enhancement of cytotoxic activity using IL-2-activated and rested human NK cells. FIG. 41A shows percent specific lysis of SkBr-3 tumor cells by rested human NK cells. FIG. 41B shows percent specific lysis of SkBr-3 tumor cells by IL-2-activated human NK cells. IL-2-activated and rested NK cell populations came from the same donor. Compared to trastuzumab, TriNKETs more potently direct responses against SkBr-3 cells by either activated or rested NK cell populations.

TriNKETs Enhance NK Cell Cytotoxicity Against Targets with Low Surface Expression

FIGS. 42A-42B show TriNKETs provide a greater advantage against HER2-medium and low cancers compared to trastuzumab. FIG. 42A shows activated human NK cell killing of HER2-high SkBr-3 tumor cells. FIG. 42B shows human NK cell killing of HER2-low 786-O tumor cells. TriNKETs provide a greater advantage compared to trastuzumab against cancer cells with low HER2 expression. TriNKETs provide the greatest advantage against targets with low surface expression.

The Advantage of TriNKETs in Treating Cancers with High Expression of FcR, or in Tumor Microenvironments with High Levels of FcR

Monoclonal antibody therapy has been approved for the treatment of many cancer types, including both hematological and solid tumors. While the use of monoclonal antibodies in cancer treatment has improved patient outcomes, there are still limitations. Mechanistic studies have demonstrated monoclonal antibodies exert their effects on tumor growth through multiple mechanisms including ADCC, CDC, phagocytosis, and signal blockade amongst others.

Most notably, ADCC is thought to be a major mechanism through which monoclonal antibodies exert their effect. ADCC relies on antibody Fc engagement of the low-affinity FcγRIII (CD16) on the surface of natural killer cells, which mediate direct lysis of the tumor cell. Amongst FcγR, CD16 has the lowest affinity for IgG Fc, FcγRI (CD64) is the high-affinity FcR, and binds about 1000 times stronger to IgG Fc than CD16.

CD64 is normally expressed on many hematopoietic lineages such as the myeloid lineage, and can be expressed on tumors derived from these cell types, such as acute myeloid leukemia (AML) Immune cells infiltrating into the tumor, such as MDSCs and monocytes, also express CD64 and are known to infiltrate the tumor microenvironment. Expression of CD64 by the tumor or in the tumor microenvironment can have a detrimental effect on monoclonal antibody therapy. Expression of CD64 in the tumor microenvironment makes it difficult for these antibodies to engage CD16 on the surface of NK cells, as the antibodies prefer to bind the high-affinity receptor. Through targeting two activating receptors on the surface of NK cells, TriNKETs may be able to overcome the detrimental effect of CD64 expression on monoclonal antibody therapy.

### FcRγI (CD64) Expression on Three AML Cell Lines

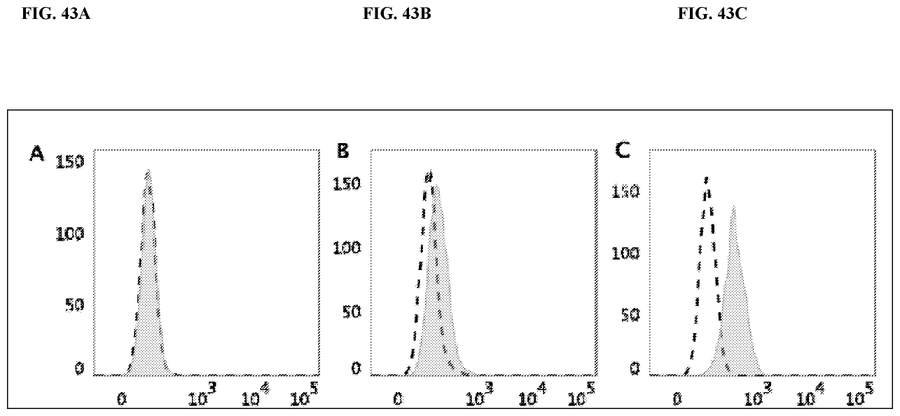

An in vitro culture system was developed to test the activity of TriNKETs and monoclonal antibodies against tumors with high and low levels of CD64 surface expression. Molm-13 and THP-1 are two human AML cell lines which have similar expression of surface CD33, but Molm-13 cells do not express CD64, while THP-1 cells express CD64 on their surface (FIGS. 43A-43C). Using monoclonal antibodies or TriNKETs directed to target CD33, the effect of CD64 expression by the tumor on monoclonal antibody or TriNKET therapy was tested. FIGS. 43A-43C show the expression of the high-affinity FcRγI (CD64) on three human AML cells lines, Molm-13 cell line (FIG. 43A), Mv4-11 cell line (FIG. 43B), and THP-1 cell line (FIG. 43C). Molm-13 cells do not express CD64, while Mv4-11 cells have a low level, and THP-1 have a high level of cell surface CD64.

TriNKETs have an Advantage in Targeting Tumor Cells with High Surface Expression of FcRs

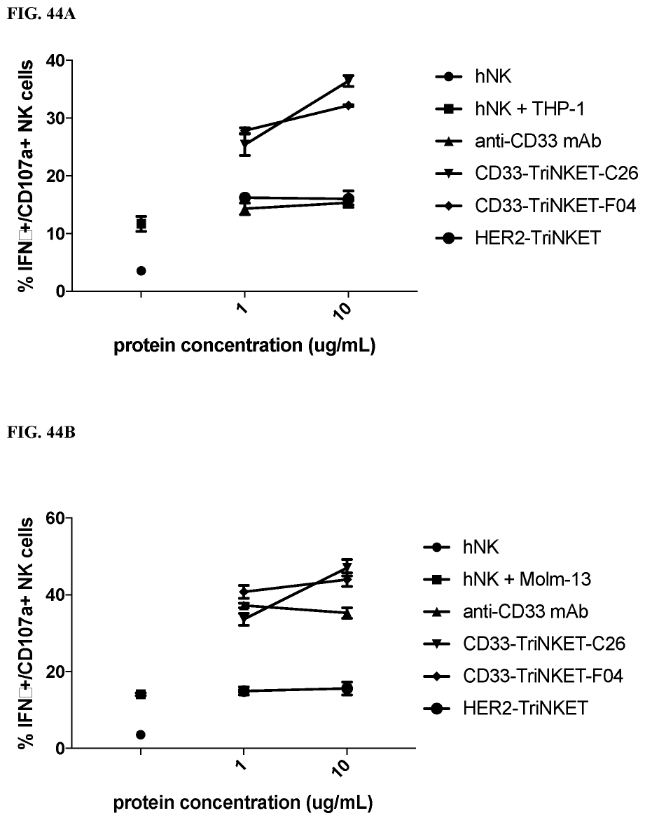

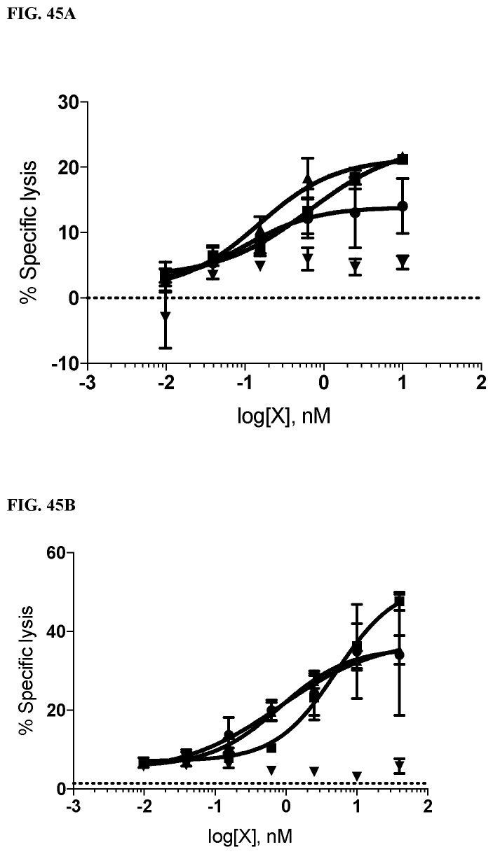

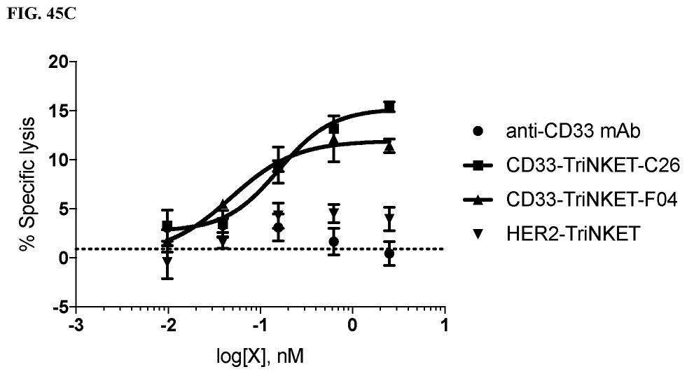

FIGS. 44A-44B show monoclonal antibody or TriNKET mediated activation of human NK cells in co-culture with either Molm-13 (FIG. 44B) or THP-1 (FIG. 44A) cells. A monoclonal antibody against human CD33 demonstrated good activation of human NK cells, in the Molm-13 co-culture system as evidenced by increased CD107a degranulation and IFNγ production. The monoclonal antibody has no effect in the THP-1 co-culture system, where high levels of CD64 are present on the tumor. Interestingly, TriNKETs were effective against both Molm-13 (FIG. 44B) and THP-1 (FIG. 44A) cells, while monoclonal antibodies fail to activate NK cells in culture with FcR-Hi THP-1 cells, indicating TriNKETs are able to overcome binding to CD64 on the tumor, and effectively target NK cells for activation. Dual targeting of two activating receptors on NK cells provided stronger specific binding to NK cells. Monoclonal antibodies, which only target CD16 on NK cells, can be bound by other high-affinity FcRs, and prevent engagement of CD16 on NK cells.

Human NK cell cytotoxicity assays using the Molm-13 and THP-1 co-culture systems provide additional evidence to support the efficacy of TriNKETs in the presence of high-levels of CD64. In these cytotoxicity assays a third human AML cell line was used, Mv4-11. Mv4-11 cells express low levels of CD64, and fall in between THP-1 and Molm-13 cells for the levels of CD64 on their surface (FIGS. 44A-44C).

### TriNKETs Demonstrate Efficacy on AML Cell Lines Regardless of FcγRI Expression

FIGS. 45A-45C show human NK cytotoxicity assays using the three human AML cell lines as targets. A monoclonal antibody against CD33 shows good efficacy against Molm-13 cells (FIG. 45B), which do not express CD64. Mv4-11 cells (FIG. 45A), which express CD64, but at a lower level than THP-1, showed reduced efficacy with the monoclonal anti-CD33. THP-1 cells (FIG. 45C) showed no effect with monoclonal anti-CD33 alone. Regardless of CD64 expression on the tumor cells, TriNKETs were able to mediate human NK cell responses against all tumor cells tested here.

FIGS. 45A-45C show that THP-1 cells were protected against monoclonal antibody therapy, due to high levels of high-affinity FcR expression on their surface. TriNKETs circumvented this protection by targeting two activating receptors on the surface of NK cells. Cytotoxicity data correlated directly to what was seen in the co-culture activation experiments. TriNKETs were able to circumvent protection from mAb therapy seen with THP-1 cells, and induce NK cell mediated lysis despite high levels of FcR.

### Killing of Normal Myeloid and Normal B Cells in PBMC Cultures: TriNKETs Provide Better Safety Profile Through Less On-Target Off-Tumor Side Effects

Natural killer cells and CD8 T cells are both able to directly lyse tumor cells, although the mechanisms through which NK cells and CD8 T cell recognize normal self from tumor cells differ. The activity of NK cells is regulated by the balance of signals from activating (NCRs, NKG2D, CD16, etc.) and inhibitory (KIRs, NKG2A, etc.) receptors. The balance of these activating and inhibitory signals allow NK cells to determine healthy self-cells from stressed, virally infected, or transformed self-cells. This ‘built-in’ mechanism of self-tolerance, will help protect normal healthy tissue from NK cell responses. To extend this principle, the self-tolerance of NK cells will allow TriNKETs to target antigens expressed both on self and tumor without off tumor side effects, or with an increased therapeutic window.

Unlike natural killer cells, T cells require recognition of a specific peptide presented by MHC molecules for activation and effector functions. T cells have been the primary target of immunotherapy, and many strategies have been developed to redirect T cell responses against the tumor. T cell bispecifics, checkpoint inhibitors, and CAR-T cells have all been approved by the FDA, but often suffer from dose-limiting toxicities. T cell bispecifics and CAR-T cells work around the TCR-MHC recognition system by using binding domains to target antigens on the surface of tumor cells, and using engineered signaling domains to transduce the activation signals into the effector cell. Although effective at eliciting an anti-tumor immune response these therapies are often coupled with cytokine release syndrome (CRS), and on-target off-tumor side effects. TriNKETs are unique in this context as they will not “override” the natural systems of NK cell activation and inhibition. Instead, TriNKETs are designed to sway the balance, and provide additional activation signals to the NK cells, while maintaining NK tolerance to healthy self.

PBMCs were isolated from whole blood by density gradient centrifugation. Any contaminating red blood cells were lysed by incubation in ACK lysis buffer. PBMCs were washed 3× in PBS, and total PBMCs were counted. PBMCs were adjusted to 10⁶/mL in primary cell culture media. 1 mL of PBMCs were seeded into wells of a 24 well plate, the indicated TriNKETs or mAbs were added to the PBMC cultures at 10 μg/mL. Cells were cultured overnight at 37° C. with 5% CO2. The following day (24 hrs later) PBMCs were harvested from culture and prepared for FACS analysis. The percentage of CD45+; CD19+ B cells and CD45+; CD33+; CD11b+myeloid cells was analyzed over the different treatment groups.

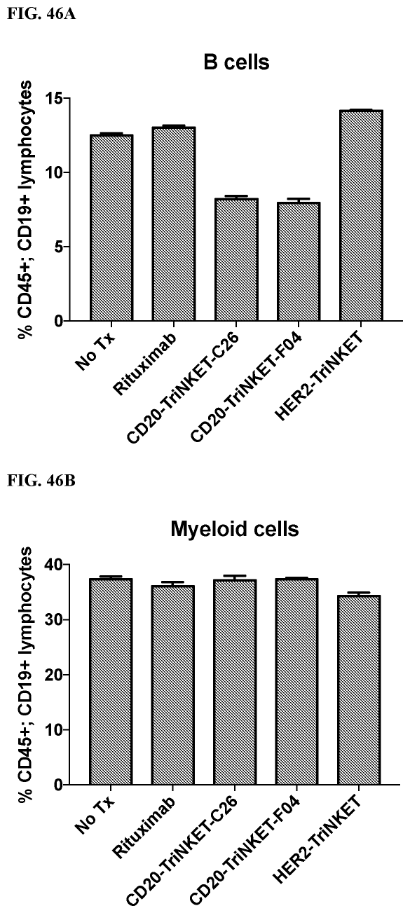

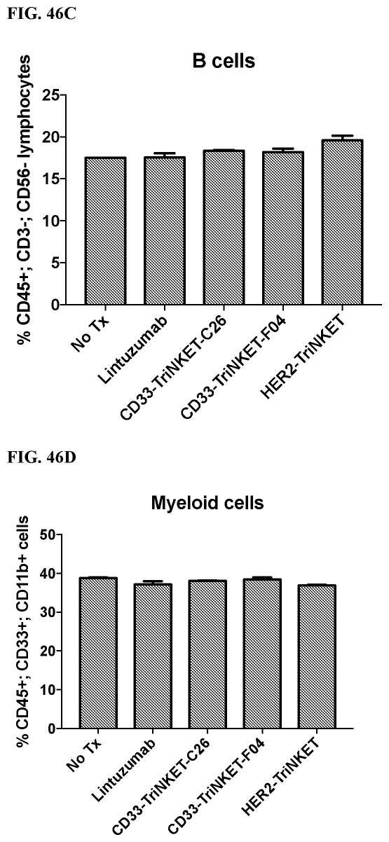

FIGS. 46B & 46D show that autologous myeloid cells are protected from TriNKET mediated NK cell responses. FIGS. 46A & 46B shows B cells from a health donor are sensitive to TriNKET mediated lysis, while myeloid cells are resistant to TriNKET lysis. PBMCs treated with TriNKETs targeting CD20 showed reduced frequency of CD19+ B cells with the CD45+ lymphocyte population (FIG. 46A), but no effect in CD45+, CDD3−, CD56− lymphocyte population (FIG. 46C). In these cultures the frequency of CD45+, CD19+ myeloid cells (FIG. 46B), or the frequency of CD33+, CD 33+, CD11b+ myeloid cells (FIG. 46D) were unchanged.

TriNKETs Mediate hPBMC Killing of SkBr-3 Tumor Cells in Long-Term Co-Cultures

### Primary Human PBMC Cytotoxicity Assay

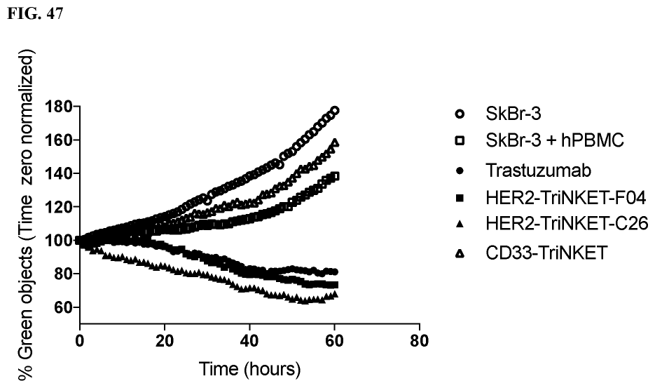

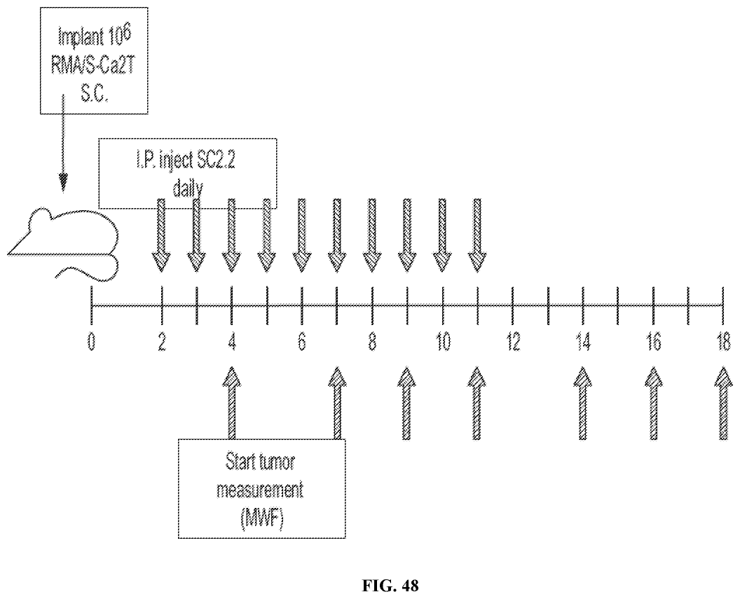

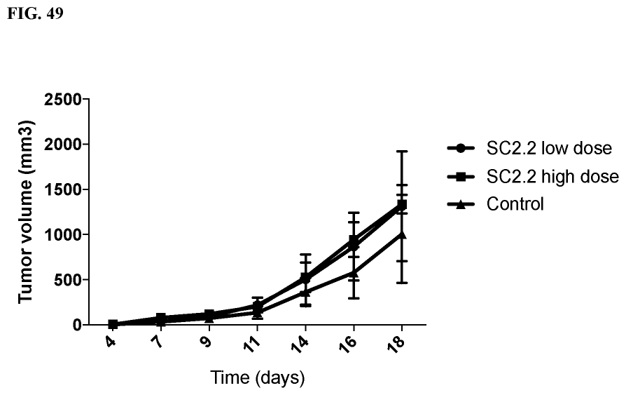

FIG. 47 shows long term killing of SkBr-3 cells in culture with human PBMCs. When cultured alone SkBr-3 cells proliferate and almost double in 60 hours. When human PBMCs are added to SkBr-3 cells in culture the rate of proliferation is slowed, and when an isotype control TriNKET targeting CD33 is added proliferation is also slowed, but to a lesser extent. When cultures are treated with Trastuzumab SkBr-3 no longer proliferate, and after 60 hours only 80% of the cells from time zero are left. Since SkBr-3 cells are sensitive to HER2 signal blockade the effect on SkBr-3 cell growth could be mediated by HER2 signal blockade or through Fc effector functions such as ADCC.

### Example 15

Anti-Tumor Efficacy of mcFAE-C26.99 TriNKETs In Vitro

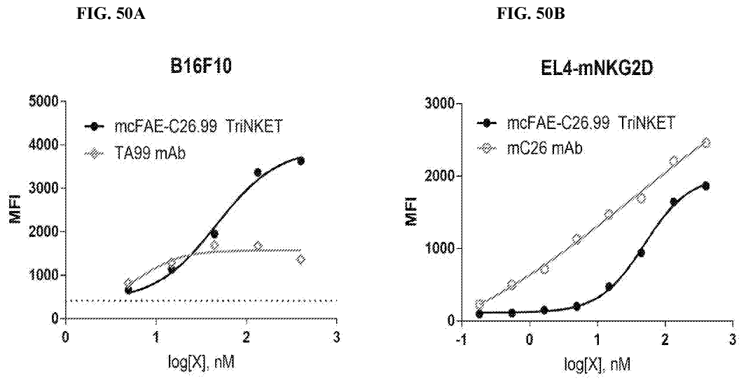

To verify binding activities of the murine cFAE-C26.99 TriNKET, direct binding was measured in comparison to its monoclonal antibodies by flow cytometry assays against Tyrp-1-positive B16F10 melanoma cells (FIG. 50A) and the EL4 line overexpressing murine NKG2D (EL4-mNKG2D, FIG. 50B).

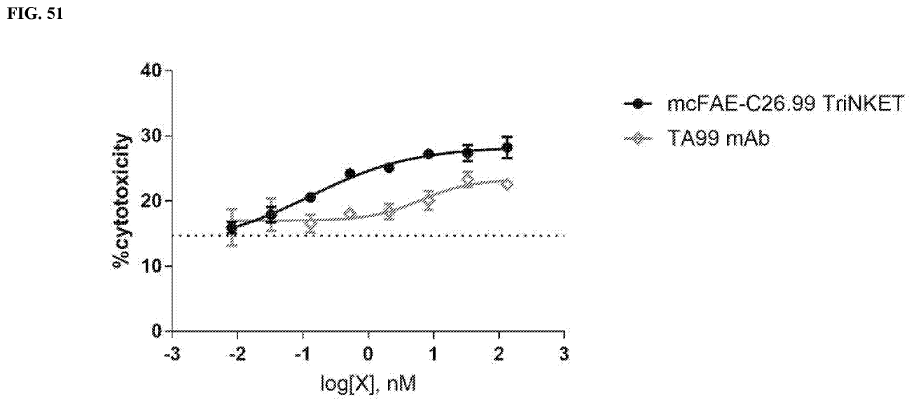

To test whether mcFAE-C26.99 TriNKETs retained the ability to mediate cytotoxicity, killing of Tyrp-1-positive B16F10 tumor targets by murine IL-2-activated NK cells was measured. As shown in FIG. 51, murine NK cells increased their cytotoxic activity in the presence of mcFAE-C26.99. Importantly, the anti-Tyrp-1 monoclonal antibody TA99 exhibited only marginal effects.

Increased NK Cytotoxicity Mediated by mcFAE-C26.99 TriNKET

About 5×10³ B 16F10 melanoma cells per well were seeded two days prior to assay. On the day of the experiment 5×10⁴ murine IL-2-activated NK cells were added in the presence of TA99 mab or mcFAE-C26.99 TriNKET (mcFAE-C26.99 is a heterodimer of mC26 and TA99 with mouse IgG2c as the Fc. Gm mutations refer to heterodimerization mutations used to generate heterodimer). 20 μg/mL of antibodies with four-fold dilutions were used. After 4 hours of co-culture, percentage of cytotoxicity was assessed using CytoTox96 kit for LDH release. Dotted line represents baseline cytotoxicity in the absence of antibodies.

mC26_hvL_mCL (Bolded Section) (Italicized Underlined Amino Acids are the Heterodimerization Mutations Used to Generate Heterodimer):

(SEQ ID NO: 49)
DIQMTQSPSTLSASVGDRVTITCRASQSISSWLAWYQQKPGKAPKLLIYK
ASSLESGVPSRFSGSGSGTEFTLTISSLQPDDFATYYCQQYGSFPITFGG
GTKVEIKRADAAPTVSIFPPSSEQLTSGGASVVCFLNNEYPKDINVKWKI
DGSERQNGVLNSWTDQDSKDSTYSMSSTLTLTKDEYERHNSYTCEATHKT
STSPIVKSFNRNEC
mC26_hvH_IgG2CGmB
(SEQ ID NO: 50)
QVQLQQWGAGLLKPSETLSLTCAVYGGSFSGYYWSWIRQPPGKGLEWIGE
IDHSGSTNYNPSLKSRVTISVDTSKNQFSLKLSSVTAADTAVYYCARARG
PWSFDPWGQGTLVTVSSAKTTAPSVYPLAPVCGGTTGSSVTLGCLVKGYF
PEPVTLTWNSGSLSSGVHTEPALLQSGLYTLSSSVTVTSNTWPSQTITCN
VAHPASSTKVDKKIEPRVPITQNPCPPLKECPPCAAPDLLGGPSVFIFPP
KIKDVLMISLSPMVTCVVVDVSEDDPDVQISWEVNNVEVHTAQTQTHRED
YNSTLRVVSALPIQHQDWMSGKEEKCKVNNRALPSPIEKTISKPRGPVRA
PQVYVLPPPAEEMTKKEFSLTCM [symbol] KGELPAEIAVDWTSNGRTEQNYKNTA
TVLDSDGSYFMYS [symbol] LRVQKSTWERGSLFACSVVHEGLHNHLTTKTISRSL
GK
TA99_mvL_mCL
(SEQ ID NO: 51)
DIQMSQSPASLSASVGETVTITCRASGNIYNYLAWYQQKQGKSPHLLVYD
AKTLADGYPSRFSGSGSGTQYSLKISSLQTEDSGNYYCQHFWSLPFTFGS
GTKLEIKRADAAPTVSIFPPSSEQLTSGGASVVCFLNNEYPKDINVKWKI
DGSERQNGVLNSWTDQDSKDSTYSMSSTLTLTKDEYERHNSYTCEATHKT
STSPIVKSFNRNEC
TA99_mvH_IgG2CGmA
(SEQ ID NO: 52)
EVQLQQSGAELVRPGALVKLSCKTSGFNIKDYFLHWVRQRPDQGLEWIGW
INPDNGNTVYDPKFQGTASLTADTSSNTVYLQLSGLTSEDTAVYFCTRRD
YTYEKAALDYWGQGASVIVSSAKTTAPSVYPLAPVCGGTTGSSVTLGCLV
KGYFPEPVTLTWNSGSLSSGVHTFPALLQSGLYTLSSSVTVTSNTWPSQT
ITCNVAHPASSTKVDKKIEPRVPITQNPCPPLKECPPCAAPDLLGGPSVF
IFPPKIKDVLMISLSPMVTCVVVDVSEDDPDVQISWEVNNVEVHTAQTQT
HREDYNSTLRVVSALPIQHQDWMSGKEEKCKVNNRALPSPIEKTISKPRG
PVRAPQVYVLPPPAEEMTKKEFSLTCMITGELPAEIAVDWTSNGRTEQNY
KNTATVLDSDGSY [symbol] MYSKL [symbol] VQKSTWERGSLFACSVVHEGLHNHLTTKTI
SRSLGK

### Example 16

Anti-Tumor Efficacy of mcFAE-C26.99 TriNKETs In Vivo

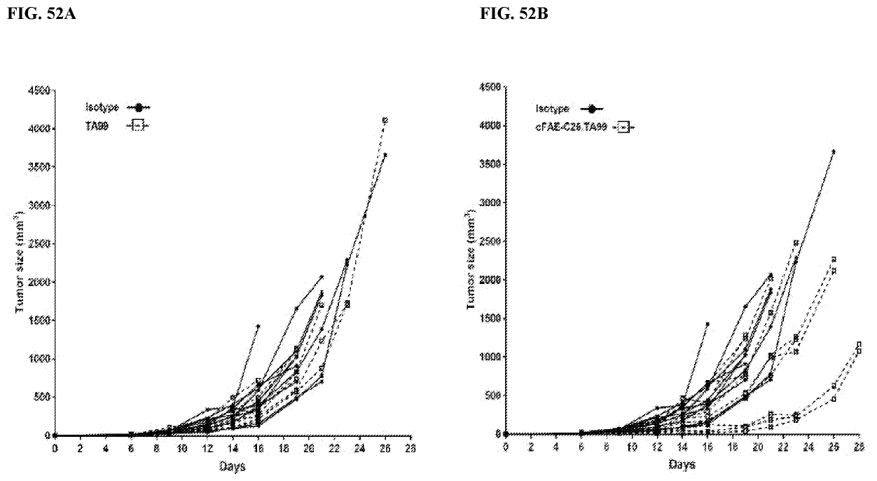

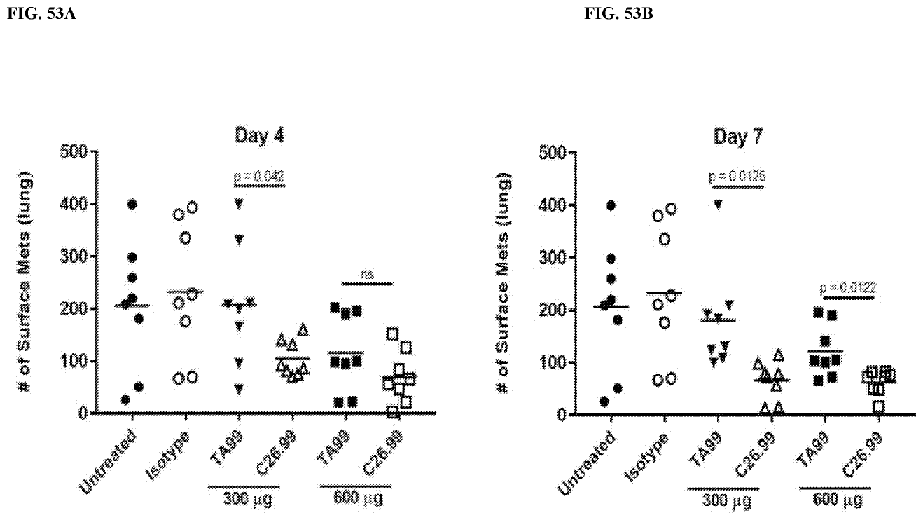

To test whether mcFAE-C26.99 elicits antitumor functions in vivo, C57BL/6 mice were injected subcutaneously with 2×10⁵ B 16F10 tumor cells. Mice were treated either with the isotype control, monoclonal TA99 antibody or with the mcFAE-C26.99 TriNKET. Treatment with the monoclonal TA99 antibody showed similar tumor progression as in the control group treated with the isotype. However, administration of the mcFAE-C26.99 TriNKET resulted in delayed tumor progression compared to the isotype-treated group. About 2×10⁵ B 16F10 melanoma cells were injected subcutaneously into the flank of C57BL/6 mice. On Day 6 after tumor inoculation mice were randomized (n=10 per group). Mice were treated intraperitoneally with (FIG. 52A) isotype control mouse IgG2a mab C1.18.4 and mouse anti-Tyrp-1 monoclonal antibody or (FIG. 52B) isotype control mouse IgG2a mab C1.18.4 and mcFAE-C26.99 TriNKET, injected at a dose of 150 μg (days 6, 8, 10, 12, 14, 16, and 21). Tumor growth was assessed for 28 days. Graphs show tumor growth curves of individual mice.

In addition to the subcutaneous B16F10 tumor model, the mcFAE-C26.99 TriNKET was also tested for its tumor efficacy in a disseminated tumor setting. 1×10⁵ B 16F10 cells were intravenously injected into mice. Treatment started either on day 4 or day 7 with a low (300 μg/injection) and high (600 μg/injection) antibody dose. On day 18 after tumor inoculation, lung metastases were counted. Treatment started at day 4 and 7 after tumor inoculation resulted in reduced numbers of lung metastases when TA99 monoclonal antibody or mcFAE-C26.99 TriNKET was used at high concentration compared to the isotype-treated control group. At low concentrations only mcFAE-C26.99 TriNKET diminished tumor burden (FIG. 53A). Similar effects were seen when antibodies were administered starting on day 7 after tumor inoculation. Overall, mcFAE-C26.99 TriNKET therapy resulted in lower numbers of lung metastases compared to the monoclonal TA99 antibody in all tested conditions. About 1×10⁵B16F10 melanoma cells were injected intravenously into the tail vein of C57BL/6 mice (n=8 per group). Mice were either left untreated or treated intraperitoneally with control mab (isotype, clone C1.18.4), monoclonal TA99 antibody or TA99 TriNKET (mcFAE-C26.99). FIG. 53A represents tumor burden when antibodies were administered at a 150-μg dose (days 4, 6, 8, 11, 13, 15). FIG. 53B represents tumor burden when antibodies were administered at a 150-μg dose (days 7, 9, 11, 13, 15). 18 days after tumor challenge, mice were euthanized and surface lung metastases were scored (FIG. 53B).

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. An antibody heavy chain variable domain comprising an amino acid sequence at least 90% identical to the amino acid sequence of SEQ ID NO:1.

2. The antibody heavy chain variable domain of claim 1, comprising an amino acid sequence at least 95% identical to the amino acid sequence of SEQ ID NO:1.

3. The antibody heavy chain variable domain according to claim 1 or 2, wherein the amino acid sequence includes:
a CDR1 sequence identical to the amino acid sequence of SEQ ID NO:2;
a CDR2 sequence identical to the amino acid sequence of SEQ ID NO:3; and
a CDR3 sequence identical to the amino acid sequence of SEQ ID NO:4.

4. The antibody heavy chain variable domain according to claim 1 or 2, wherein the amino acid sequence includes:
a CDR1 sequence identical to the amino acid sequence of SEQ ID NO:2;
a CDR2 sequence identical to the amino acid sequence of SEQ ID NO:3; and
a CDR3 sequence identical to the amino acid sequence of SEQ ID NO:5.

5. An antibody heavy chain comprising an antibody heavy chain variable domain according to any one of claims 1-4 and an amino acid sequence at least 90% identical to an antibody constant region.

6. The antibody heavy chain of claim 5, wherein the antibody constant region is a human IgG constant region comprising hinge, CH2 and CH3 domains.

7. The antibody heavy chain of claim 6, wherein the antibody constant region is a human IgG constant region further comprises a CH1 domain.

8. An antibody heavy chain according to any of claims 5-7, wherein the antibody constant region is an IgG1 constant region.

9. An antibody heavy chain according to claim 8 wherein the amino acid sequence at least 90% identical to an antibody constant region differs from the amino acid sequence of an IgG1 constant region at one or more positions selected from the group consisting of Q347, Y349, L351, S354, E356, E357, K360, Q362, S364, T366, L368, K370, N390, K392, T394, D399, S400, D401, F405, Y407, K409, T411, K439.

10. The antibody heavy chain of claim 9 wherein the amino acid sequence at least 90% identical to an antibody constant region differs from the amino acid sequence of an IgG1 constant region by one or substitutions selected from the group consisting of Q347E, Q347R, Y349S, Y349K, Y349T, Y349D, Y349E, Y349C, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, D399R, D399K, D399V, S400K, S400R, D401K, F405A, F405T, Y407A, Y407I, Y407V, K409F, K409W, K409D, T411D, T411E, K439D, and K439E.

11. An antigen-binding site comprising an antibody heavy chain variable domain according to any one of claims 1-4 and an antibody light chain variable domain.

12. The antigen-binding site of claim 11, wherein the antigen-binding site binds NKG2D.

13. A protein comprising:
a first antigen-binding site comprising a first antibody heavy chain variable domain according to any one of claims 1-4 and a first antibody light chain variable domain; and
a second antigen-binding site comprising a second antibody heavy chain variable domain and a second antibody light chain variable domain.

14. The protein of claim 13, wherein the first antigen-binding site binds NKG2D.

15. A protein according to claim 13 or 14, wherein the second antigen-binding site binds to a tumor-associated antigen.

16. The protein of claim 15, wherein the tumor-associated antigen is selected from the group consisting of EpCAM, CD33, HER2, CD2, CD19, BCMA, CD20, CD30, CD38, CD40, CD52, CD70, EGFR/ERBB1, IGF1R, HER3/ERBB3, HER4/ERBB4, MUC1, cMET, SLAMF7, PSCA, MICA, MICB, TRAILR1, TRAILR2, MAGE-A3, B7.1, B7.2, CTLA4, and PD1.

17. A protein according to any one of claims 13-16, wherein the amino acid sequence of the first antibody light chain variable domain is identical to the amino acid sequence of the second antibody light chain variable domain.

18. A protein according to any one of claims 13-16, wherein the first antibody heavy chain variable domain is present on a first polypeptide further comprising an amino acid sequence at least 90% identical to a first antibody constant region, and wherein the second antibody heavy chain variable domain is present on a second polypeptide further comprising an amino acid sequence at least 90% identical to a second antibody constant region.

19. The protein of claim 18, wherein the first antibody constant region and the second antibody constant region each comprise hinge, CH2 and CH3 domains.

20. The protein of claim 19, wherein the first antibody constant region and the second antibody constant region each comprise CH1, hinge, CH2 and CH3 domains.

21. A protein according to any one of claims 18-20, wherein the first antibody constant region and the second antibody constant region are each human IgG1 constant regions.

22. The protein of claim 21, wherein:
the amino acid sequence at least 90% identical to a first antibody constant region differs from the amino acid sequence of an IgG1 constant region at one or more positions selected from the group consisting of Q347, Y349, L351, S354, E356, E357, K360, Q362, 5364, T366, L368, K370, K392, T394, D399, S400, D401, F405, Y407, K409, T411 and K439; and
the amino acid sequence at least 90% identical to a second antibody constant region differs from the amino acid sequence of an IgG1 constant region at one or more positions selected from the group consisting of Q347, Y349, L351, S354, E356, E357, S364, T366, L368, K370, N390, K392, T394, D399, D401, F405, Y407, K409, T411 and K439.

23. The protein of claim 21, wherein the amino acid sequence at least 90% identical to a first antibody constant region differs from the amino acid sequence of an IgG1 constant region at position T366, and wherein the amino acid sequence at least 90% identical to a second antibody constant region differs from the amino acid sequence of an IgG1 constant region at one or more positions selected from the group consisting of T366, L368 and Y407.

24. The protein of claim 21, wherein the amino acid sequence at least 90% identical to a first antibody constant region differs from the amino acid sequence of an IgG1 constant region at one or more positions selected from the group consisting of T366, L368 and Y407, and wherein the amino acid sequence at least 90% identical to a second antibody constant region differs from the amino acid sequence of an IgG1 constant region at position T366.

25. The protein of claim 21, wherein the amino acid sequence at least 90% identical to a first antibody constant region differs from the amino acid sequence of an IgG1 constant region at one or more positions selected from the group consisting of E357, K360, Q362, S364, L368, K370, T394, D401, F405, and T411 and wherein the amino acid sequence at least 90% identical to a second antibody constant region differs from the amino acid sequence of an IgG1 constant region at one or more positions selected from the group consisting of Y349, E357, S364, L368, K370, T394, D401, F405 and T411.

26. The protein of claim 21, wherein the amino acid sequence at least 90% identical to a first antibody constant region differs from the amino acid sequence of an IgG1 constant region at one or more positions selected from the group consisting of Y349, E357, S364, L368, K370, T394, D401, F405 and T411 and wherein the amino acid sequence at least 90% identical to a second antibody constant region differs from the amino acid sequence of an IgG1 constant region at one or more positions selected from the group consisting of E357, K360, Q362, S364, L368, K370, T394, D401, F405, and T411.

27. The protein of claim 21, wherein the amino acid sequence at least 90% identical to a first antibody constant region differs from the amino acid sequence of an IgG1 constant region at one or more positions selected from the group consisting of L351, D399, S400 and Y407 and wherein the amino acid sequence at least 90% identical to a second antibody constant region differs from the amino acid sequence of an IgG1 constant region at one or more positions selected from the group consisting of T366, N390, K392, K409 and T411.

28. The protein of claim 21, wherein the amino acid sequence at least 90% identical to a first antibody constant region differs from the amino acid sequence of an IgG1 constant region at one or more positions selected from the group consisting of T366, N390, K392, K409 and T411 and wherein the amino acid sequence at least 90% identical to a second antibody constant region differs from the amino acid sequence of an IgG1 constant region at one or more positions selected from the group consisting of L351, D399, S400 and Y407.

29. The protein of claim 21, wherein the amino acid sequence at least 90% identical to a first antibody constant region differs from the amino acid sequence of an IgG1 constant region at one or more positions selected from the group consisting of Q347, Y349, K360, and K409, and wherein the amino acid sequence at least 90% identical to a second antibody constant region differs from the amino acid sequence of an IgG1 constant region at one or more positions selected from the group consisting of Q347, E357, D399 and F405.

30. The protein of claim 21, wherein the amino acid sequence at least 90% identical to a first antibody constant region differs from the amino acid sequence of an IgG1 constant region at one or more positions selected from the group consisting of Q347, E357, D399 and F405, and wherein the amino acid sequence at least 90% identical to a second antibody constant region differs from the amino acid sequence of an IgG1 constant region at one or more positions selected from the group consisting of Y349, K360, Q347 and K409.

31. The protein of claim 21, wherein the amino acid sequence at least 90% identical to a first antibody constant region differs from the amino acid sequence of an IgG1 constant region at one or more positions selected from the group consisting of K370, K392, K409 and K439, and wherein the amino acid sequence at least 90% identical to a second antibody constant region differs from the amino acid sequence of an IgG1 constant region at one or more positions selected from the group consisting of D356, E357 and D399.

32. The protein of claim 21, wherein the amino acid sequence at least 90% identical to a first antibody constant region differs from the amino acid sequence of an IgG1 constant region at one or more positions selected from the group consisting of D356, E357 and D399, and wherein the amino acid sequence at least 90% identical to a second antibody constant region differs from the amino acid sequence of an IgG1 constant region at one or more positions selected from the group consisting of K370, K392, K409 and K439.

33. The protein of claim 21, wherein the amino acid sequence at least 90% identical to a first antibody constant region differs from the amino acid sequence of an IgG1 constant region at one or more positions selected from the group consisting of L351, E356, T366 and D399, and wherein the amino acid sequence at least 90% identical to a second antibody constant region differs from the amino acid sequence of an IgG1 constant region at one or more positions selected from the group consisting of Y349, L351, L368, K392 and K409.

34. The protein of claim 21, wherein the amino acid sequence at least 90% identical to a first antibody constant region differs from the amino acid sequence of an IgG1 constant region at one or more positions selected from the group consisting of Y349, L351, L368, K392 and K409, and wherein the amino acid sequence at least 90% identical to a second antibody constant region differs from the amino acid sequence of an IgG1 constant region at one or more positions selected from the group consisting of L351, E356, T366 and D399.

35. The protein of claim 21, wherein the amino acid sequence at least 90% identical to a first antibody constant region differs from the amino acid sequence of an IgG1 constant region by an S354C substitution and wherein the amino acid sequence at least 90% identical to a second antibody constant region differs from the amino acid sequence of an IgG1 constant region by a Y349C substitution.

36. The protein of claim 21, wherein the amino acid sequence at least 90% identical to a first antibody constant region differs from the amino acid sequence of an IgG1 constant region by a Y349C substitution and wherein the amino acid sequence at least 90% identical to a second antibody constant region differs from the amino acid sequence of an IgG1 constant region by an S354C substitution.

37. The protein of claim 21, wherein the amino acid sequence at least 90% identical to a first antibody constant region differs from the amino acid sequence of an IgG1 constant region by an S354C substitution and wherein the amino acid sequence at least 90% identical to a second antibody constant region differs from the amino acid sequence of an IgG1 constant region by a Y349C substitution.

38. The protein of claim 21, wherein the amino acid sequence at least 90% identical to a first antibody constant region differs from the amino acid sequence of an IgG1 constant region by a Y349C substitution and wherein the amino acid sequence at least 90% identical to a second antibody constant region differs from the amino acid sequence of an IgG1 constant region by an S354C substitution.

39. A protein comprising a polypeptide having the amino acid sequence at least 90% identical to the amino acid sequence of SEQ ID NO:33.

40. A protein comprising a polypeptide having the amino acid sequence at least 95% identical to the amino acid sequence of SEQ ID NO:33.

41. A protein comprising a polypeptide having the amino acid sequence of SEQ ID NO:33.

42. The protein according to any one of claims 39-41, further comprising an antibody light chain variable region.

43. The protein according to any one of claims 39-41, further comprising a second antibody heavy chain and a second antibody light chain variable region.

44. A protein comprising a polypeptide having the amino acid sequence at least 90% identical to the amino acid sequence of SEQ ID NO:35.

45. A protein comprising a polypeptide having the amino acid sequence at least 95% identical to the amino acid sequence of SEQ ID NO:35.

46. A protein comprising a polypeptide having the amino acid sequence of SEQ ID NO:35.

47. The protein according to any one of claims 44-46, further comprising an antibody light chain variable region.

48. The protein according to any one of claims 44-46, further comprising a second antibody heavy chain and a second antibody light chain variable region.

49. A protein comprising an antigen-binding site that competes for binding to mouse and human NKG2D with an antibody comprising an antibody heavy chain having the amino acid sequence of SEQ ID NO:6 and an antibody light chain having the amino acid sequence of SEQ ID NO:7.

50. A protein comprising an antigen-binding site that competes for binding to mouse and human NKG2D with an antibody comprising an antibody heavy chain having the amino acid sequence of SEQ ID NO:8 and an antibody light chain having the amino acid sequence of SEQ ID NO:9.

51. A protein comprising an antigen-binding site that competes for binding to mouse and human NKG2D with an antibody comprising an antibody heavy chain having the amino acid sequence of SEQ ID NO:10 and an antibody light chain having the amino acid sequence of SEQ ID NO:11.

52. A protein according to any one of claims 49-51, wherein the antigen-binding site also binds to cynomolgus monkey NKG2D.

53. A protein according to any one of claims 49-51, further comprising an antigen-binding site that binds a tumor-associated antigen.

54. A protein according to any one of claims 49-53, wherein the protein includes an antibody constant region or a portion thereof capable of binding CD16.

55. The protein of claim 54, wherein the antibody constant region is a human IgG1 constant region.

56. A protein according to any one of claims 49-53, wherein the protein includes an antibody variable region capable of binding CD16.

57. A formulation comprising a protein according to any one of claims 13-56 and a pharmaceutically acceptable carrier.

58. A cell comprising one or more nucleic acids encoding a protein according to any one of claims 13-56.

59. A method of enhancing tumor cell death, the method comprising exposing a tumor and natural killer cells to a protein according to any one of claims 13-56.

60. A method of treating cancer, wherein the method comprises administering a protein according to any one of claims 13-56 or a formulation according to claim 57 to a patient.

61. The method of claim 60, wherein the cancer is selected from the group consisting of acute myeloid leukemia, acute myelomonocytic leukemia, B cell lymphoma, bladder cancer, breast cancer, colorectal cancer, diffuse large B cell lymphoma esophageal cancer, Ewing's sarcoma, follicular lymphoma, gastric cancer, gastrointestinal cancer, gastrointestinal stromal tumors, glioblastoma, head and neck cancer, melanoma, mesothelioma, multiple myeloma, myelodysplastic syndrome, renal cell carcinoma, neuroblastoma, non-small cell lung cancer, neuroendocrine tumors, ovarian cancer, and pancreatic cancer, prostate cancer, sarcomas, small cell lung cancer, T cell lymphoma, testis cancer, thymic carcinoma, thyroid cancer, urothelial cancer, cancers infiltrated by myeloid-derived suppressor cells, cancers with extracellular matrix deposition, cancers with high levels of reactive stroma, and cancers with neoangiogenesis.


SEQUENCE LISTING (52 sequences)

SEQ ID NO: 1 - protein, 117 aa, Artificial Sequence
  Description of Artificial Sequence Synthetic polypeptide
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSS

SEQ ID NO: 2 - protein, 9 aa, Artificial Sequence
  Description of Artificial Sequence Synthetic peptide
GSFSGYYWS

SEQ ID NO: 3 - protein, 16 aa, Artificial Sequence
  Description of Artificial Sequence Synthetic peptide
EIDHSGSTNYNPSLKS

SEQ ID NO: 4 - protein, 11 aa, Artificial Sequence
  Description of Artificial Sequence Synthetic peptide
ARARGPWSFDP

SEQ ID NO: 5 - protein, 11 aa, Artificial Sequence
  Description of Artificial Sequence Synthetic peptide
ARARGPWGFDP

SEQ ID NO: 6 - protein, 447 aa, Artificial Sequence
  Description of Artificial Sequence Synthetic polypeptide
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 214 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSYPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 451 aa, Artificial Sequence
  Description of Artificial Sequence Synthetic polypeptide
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARGSDRFHPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 214 aa, Artificial Sequence
  Description of Artificial Sequence Synthetic polypeptide
EIVLTQSPATLSLSPGERATLSCRASQSVSRYLAWYQQKPGQAPRLLIYDASNRATGIPARFSGSGSGTDFTLTISSLEPEDFAVYYCQQFDTWPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 455 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGDSSIRHAYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 220 aa, Artificial Sequence
  Description of Artificial Sequence Synthetic polypeptide
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 107 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSYPITFGGGTKVEIK

SEQ ID NO: 13 - protein, 108 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPITFGGGTKVEIK

SEQ ID NO: 14 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYHSFYTFGGGTKVEIK

SEQ ID NO: 15 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQSNSYYTFGGGTKVEIK

SEQ ID NO: 16 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSYPTFGGGTKVEIK

SEQ ID NO: 17 - protein, 117 aa, Artificial Sequence
  Description of Artificial Sequence Synthetic polypeptide
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWGFDPWGQGTLVTVSS

SEQ ID NO: 18 - protein, 107 aa, Artificial Sequence
  Description of Artificial Sequence Synthetic polypeptide
ELQMTQSPSSLSASVGDRVTITCRTSQSISSYLNWYQQKPGQPPKLLIYWASTRESGVPDRFSGSGSGTDFTLTISSLQPEDSATYYCQQSYDIPYTFGQGTKLEIK

SEQ ID NO: 19 - protein, 107 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYGSFPITFGGGTKVEIK

SEQ ID NO: 20 - protein, 107 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTDFTLTISSLQPDDFATYYCQQSKEVPWTFGQGTKVEIK

SEQ ID NO: 21 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSFPTFGGGTKVEIK

SEQ ID NO: 22 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYDIYPTFGGGTKVEIK

SEQ ID NO: 23 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDSYPTFGGGTKVEIK

SEQ ID NO: 24 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYGSFPTFGGGTKVEIK

SEQ ID NO: 25 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYQSFPTFGGGTKVEIK

SEQ ID NO: 26 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYSSFSTFGGGTKVEIK

SEQ ID NO: 27 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYESYSTFGGGTKVEIK

SEQ ID NO: 28 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDSFITFGGGTKVEIK

SEQ ID NO: 29 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYQSYPTFGGGTKVEIK

SEQ ID NO: 30 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYHSFPTFGGGTKVEIK

SEQ ID NO: 31 - protein, 107 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYELYSYTFGGGTKVEIK

SEQ ID NO: 32 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDTFITFGGGTKVEIK

SEQ ID NO: 33 - protein, 121 aa, Artificial Sequence
  Description of Artificial Sequence Synthetic polypeptide
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARGSDRFHPYFDYWGQGTLVTVSS

SEQ ID NO: 34 - protein, 107 aa, Artificial Sequence
  Description of Artificial Sequence Synthetic polypeptide
EIVLTQSPATLSLSPGERATLSCRASQSVSRYLAWYQQKPGQAPRLLIYDASNRATGIPARFSGSGSGTDFTLTISSLEPEDFAVYYCQQFDTWPPTFGGGTKVEIK

SEQ ID NO: 35 - protein, 125 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGDSSIRHAYYYYGMDVWGQGTTVTVSS

SEQ ID NO: 36 - protein, 113 aa, Artificial Sequence
  Description of Artificial Sequence Synthetic polypeptide
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPITFGGGTKVEIK

SEQ ID NO: 37 - protein, 110 aa, Homo sapiens
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 38 - protein, 121 aa, Homo sapiens
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 39 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 40 - protein, 115 aa, Homo sapiens
QVHLQESGPGLVKPSETLSLTCTVSDDSISSYYWSWIRQPPGKGLEWIGHISYSGSANYNPSLKSRVTISVDTSKNQFSLKLSSVTAADTAVYYCANWDDAFNIWGQGTMVTVSS

SEQ ID NO: 41 - protein, 246 aa, Artificial Sequence
  Description of Artificial Sequence Synthetic polypeptide
MAAAAIPALLLCLPLLFLLFGWSRARRDDPHSLCYDITVIPKFRPGPRWCAVQGQVDEKTFLHYDCGNKTVTPVSPLGKKLNVTMAWKAQNPVLREVVDILTEQLLDIQLENYTPKEPLTLQARMSCEQKAEGHSSGSWQFSIDGQTFLLFDSEKRMWTTVHPGARKMKEKWENDKDVAMSFHYISMGDCIGWLEDFLMGMDSTLEPSAGAPLAMSSGTTQLRATATTLILCCLLIILPCFILPGI

SEQ ID NO: 42 - protein, 120 aa, Artificial Sequence
  Description of Artificial Sequence Synthetic polypeptide
QVQLVQSGAEVKKPGASVKVSCKASGYTFTDYVVHWVRQAPGQGLEWMGYINPYNDGTKYNEKFKGRVTMTRDTSISTAYMELSRLRSDDTAVYYCARDYRYEVYGMDYWGQGTLVTVSS

SEQ ID NO: 43 - protein, 106 aa, Artificial Sequence
  Description of Artificial Sequence Synthetic polypeptide
DIVLTQSPASLAVSPGQRATITCTASSSVNYIHWYQQKPGQPPKLLIYDTSKVASGVPARFSGSGSGTDFTLTINPVEANDTANYYCQQWRSYPLTFGQGTKLEIK

SEQ ID NO: 44 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCEQYDSYPTFGGGTKVEIK

SEQ ID NO: 45 - protein, 126 aa, Artificial Sequence
  Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARRGRKASGSFYYYYGMDVWGQGTTVTVSS

SEQ ID NO: 46 - protein, 113 aa, Artificial Sequence [sequence omitted]

SEQ ID NO: 47 - protein, 121 aa, Artificial Sequence [sequence omitted]

SEQ ID NO: 48 - protein, 106 aa, Artificial Sequence [sequence omitted]

SEQ ID NO: 49 - protein, 214 aa, Artificial Sequence
  Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYGSFPITFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 50 - protein, 452 aa, Artificial Sequence
  Synthetic polypeptide
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSSAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMIKGFLPAEIAVDWTSNGRTEQNYKNTATVLDSDGSYFMYSRLRVQKSTWERGSLFACSVVHEGLHNHLTTKTISRSLGK

SEQ ID NO: 51 - protein, 214 aa, Artificial Sequence
  Synthetic polypeptide
DIQMSQSPASLSASVGETVTITCRASGNIYNYLAWYQQKQGKSPHLLVYDAKTLADGVPSRFSGSGSGTQYSLKISSLQTEDSGNYYCQHFWSLPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 52 - protein, 456 aa, Artificial Sequence
  Synthetic polypeptide
EVQLQQSGAELVRPGALVKLSCKTSGFNIKDYFLHWVRQRPDQGLEWIGWINPDNGNTVYDPKFQGTASLTADTSSNTVYLQLSGLTSEDTAVYFCTRRDYTYEKAALDYWGQGASVIVSSAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYLMYSKLTVQKSTWERGSLFACSVVHEGLHNHLTTKTISRSLGK